(19) United States
(12) Patent Application Publication
(10) Pub. No.: US 2020/0262787 A1
(43) Pub. Date: Aug. 20, 2020

(54) OPTICAL COMPONENT FORMING COMPOSITION AND CURED PRODUCT THEREOF

(71) Applicant: Mitsubishi Gas Chemical Company, Inc., Tokyo (JP)

(72) Inventors: Masatoshi ECHIGO, Chiyoda-ku, Tokyo (JP); Tomoaki TAKIGAWA, Hiratsuka-shi, Kanagawa (JP); Takumi TOIDA, Hiratsuka-shi, Kanagawa (JP); Takashi SATO, Hiratsuka-shi, Kanagawa (JP)

(21) Appl. No.: 16/096,645

(22) PCT Filed: Apr. 28, 2017
(86) PCT No.: PCT/JP2017/017092

§ 371 Date: Oct. 25, 2018

(30) Foreign Application Priority Data
Apr. 28, 2016 (JP) 2016-091792

(51) Int. Cl. C07C 395/00; C08G 79/00; G03F 7/038; G03F 7/039; G03F 7/00; G02B 1/04

(52) U.S. Cl. CPC C07C 395/00; C08G 79/00; G02B 1/04; G03F 7/039; G03F 7/0005; G03F 7/038

(57) ABSTRACT

The present invention provides an optical component forming composition comprising a tellurium-containing compound or a tellurium-containing resin.

### TECHNICAL FIELD

The present invention relates to an optical component forming composition and a cured product thereof.

### BACKGROUND ART

In recent years, various optical component forming compositions have been proposed. Examples of such an optical component forming composition include those comprising an acrylic resin, an epoxy based resin or an anthracene derivative (see, for example, Patent Literatures 1 to 4).

Meanwhile tellurium-containing polymers have been proposed (see Non Patent Literatures 1 to 3).

### CITATION LIST

### Patent Literature

Patent Literature 1: Japanese Patent Application Laid-Open No. 2016-12061
Patent Literature 2: Japanese Patent Application Laid-Open No. 2015-174877
Patent Literature 3: Japanese Patent Application Laid-Open No. 2014-73986
Patent Literature 4: Japanese Patent Application Laid-Open No. 2010-138393

### Non Patent Literature

Non Patent Literature 1: Chem. Lett., 40, 762-764 (2011)
Non Patent Literature 2: Angew. Chem. Int. Ed. 49, 10140-10144 (2010)
Non Patent Literature 3: Org. Lett., 11, 1487-1490 (2009)

### SUMMARY OF INVENTION

### Technical Problem

Although a large number of compositions intended for optical members have heretofore been proposed, none of these compositions achieve all of storage stability, the ability to form a structure (the ability to form a film), heat resistance, transparency and refractive index at high dimensions. Thus, the development of novel materials is demanded.

As mentioned above, tellurium-containing polymers have been proposed in Non Patent Literatures 1 to 3. However, none of these literatures suggest that the polymers are applied to optical component forming compositions.

An object of the present invention is to provide an optical component forming composition which is usefully used in optical materials, and a cured product thereof.

More specifically, the present invention is as follows.

<1> An optical component forming composition comprising a tellurium-containing compound or a tellurium-containing resin.
<2> The optical component forming composition according to <1>, wherein the tellurium-containing compound is represented by the following formula (A-1):

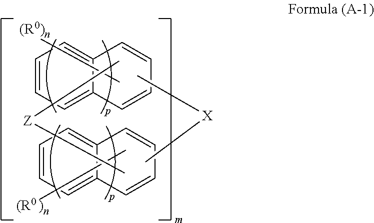

wherein X is a 2m-valent group of 0 to 60 carbon atoms containing tellurium; Z is an oxygen atom, a sulfur atom, or non-crosslinked state; each R⁰ is independently selected from the group consisting of a monovalent group containing an oxygen atom, a monovalent group containing a sulfur atom, a monovalent group containing a nitrogen atom, a hydrocarbon group, a halogen atom, and a combination thereof; m is an integer of 1 to 4; each p is independently an integer of 0 to 2; and each n is independently an integer of 0 to (5+2×p).

<3> The optical component forming composition according to <2>, wherein the tellurium-containing compound is represented by the following formula (A-2):

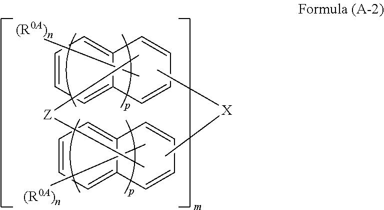

wherein X is a 2m-valent group of 0 to 60 carbon atoms containing tellurium; Z is an oxygen atom, a sulfur atom, a single bond, or non-crosslinked statenon-crosslinked state; each R^{0A} is independently selected from the group consisting of a hydrocarbon group, a halogen atom, a cyano group, a nitro group, an amino group, an alkyl group of 1 to 30 carbon atoms, an alkenyl group of 2 to 30 carbon atoms, an aryl group of 6 to 40 carbon atoms, a hydroxy group or a group in which a hydrogen atom of a hydroxy group is substituted with an acid crosslinking reactive group or an acid dissociation reactive group, and a combination thereof, wherein the alkyl group, the alkenyl group, and the aryl group each optionally have an ether bond, a ketone bond, or an ester bond; m is an integer of 1 to 4; each p is independently an integer of 0 to 2; and each n is independently an integer of 0 to (5+2×p).

<4> The optical component forming composition according to <2>, wherein the tellurium-containing compound is represented by the following formula (A-3):

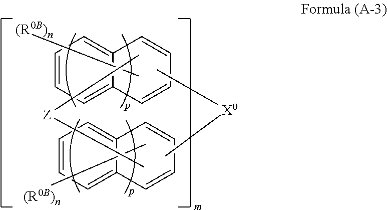

wherein X⁰ is a 2m-valent group of 0 to 30 carbon atoms containing tellurium; Z is an oxygen atom, a sulfur atom, or non-crosslinked state; each R^{0B} is independently a monovalent group containing an oxygen atom, a monovalent group containing a sulfur atom, a monovalent group containing a nitrogen atom, a hydrocarbon group, or a halogen atom; m is an integer of 1 to 4; each p is independently an integer of 0 to 2; and each n is independently an integer of 0 to (5+2×p).

<5> The optical component forming composition according to <2>, wherein the tellurium-containing compound is represented by the following formula (1A):

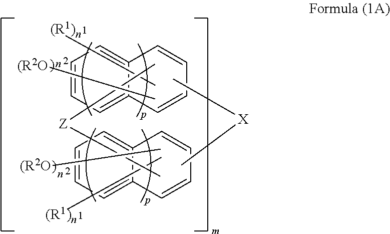

wherein X, Z, m, and p are as defined in the above formula (A-1); each R¹ is independently selected from the group consisting of a hydrocarbon group, a halogen atom, a cyano group, a nitro group, an amino group, an alkyl group of 1 to 30 carbon atoms, an alkenyl group of 2 to 30 carbon atoms, an aryl group of 6 to 40 carbon atoms, and a combination thereof, wherein the alkyl group, the alkenyl group, and the aryl group each optionally have an ether bond, a ketone bond, or an ester bond; each R² is independently a hydrogen atom, an acid crosslinking reactive group, or an acid dissociation reactive group; each n¹ is independently an integer of 0 to (5+2×p); and each n² is independently an integer of 0 to (5+2×p), provided that at least one n² is an integer of 1 to (5+2×p).

<6> The optical component forming composition according to <4>, wherein the tellurium-containing compound is represented by the following formula (1B):

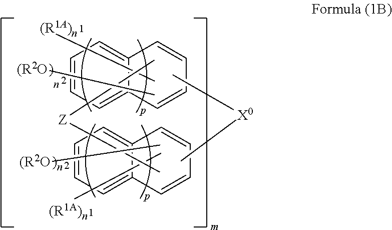

wherein X⁰, Z, m, and p are as defined in the above formula (A-3); each R^{1A} is independently an alkyl group, an aryl group, an alkenyl group, or a halogen atom; each R² is independently a hydrogen atom, an acid crosslinking reactive group, or an acid dissociation reactive group; each n¹ is independently an integer of 0 to (5+2×p); and each n² is independently an integer of 0 to (5+2×p), provided that at least one n² is an integer of 1 to (5+2×p).

<7> The optical component forming composition according to <6>, wherein the tellurium-containing compound is represented by the following formula (2A):

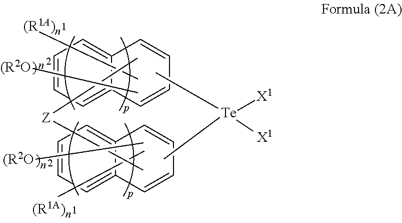

wherein Z, R^{1A}, R², p, n¹, and n² are as defined in the above formula (1B); and each X¹ is independently a monovalent group containing an oxygen atom, a monovalent group containing a sulfur atom, a monovalent group containing a nitrogen atom, a hydrocarbon group, a hydrogen atom, or a halogen atom.

<8> The optical component forming composition according to <7>, wherein the tellurium-containing compound is represented by the following formula (2A′):

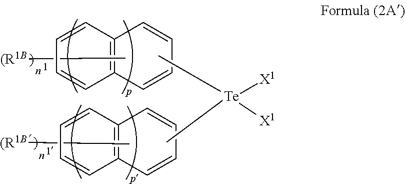

wherein R^{1B} and R^{1B′} are each independently an alkyl group, an aryl group, an alkenyl group, a halogen atom, a hydroxy group or a group in which a hydrogen atom of a hydroxy group is substituted with an acid crosslinking reactive group or an acid dissociation reactive group; X¹ is as defined as X¹ in the above formula (2A); n¹ and n^{1′} are as defined as n¹ in the above formula (2A); p and p′ are as defined as p in the above formula (2A); and members in at least one combination selected from R^{1B} and R^{1B′}, n¹ and n^{1′}, p and p′, and the substitution position of R^{1B} and the substitution position of R^{1B′} differ from each other.

<9> The optical component forming composition according to <7>, wherein the tellurium-containing compound is represented by the following formula (3A):

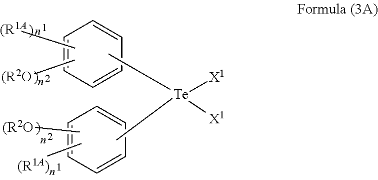

wherein R^{1A}, R², X¹, n¹, and n² are as defined in the above formula (2A).

<10> The optical component forming composition according to <9>, wherein the tellurium-containing compound is represented by the following formula (4A):

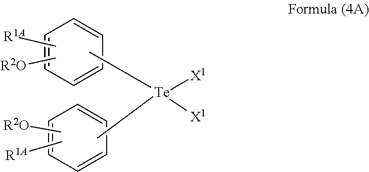

wherein R^{1A}, R², and X¹ are as defined in the above formula (3A).

<11> The optical component forming composition according to <6>, wherein the tellurium-containing compound is represented by the following formula (2B):

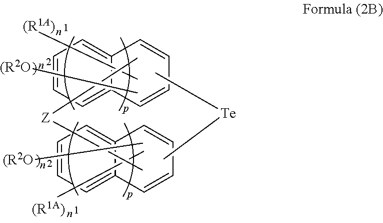

wherein Z, R^{1A}, R², p, n¹, and n² are as defined in the above formula (1B).

<12> The optical component forming composition according to <11>, wherein the tellurium-containing compound is represented by the following formula (2B′):

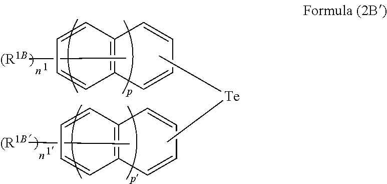

wherein R^{1B} and R^{1B′} are each independently an alkyl group, an aryl group, an alkenyl group, a halogen atom, a hydroxy group or a group in which a hydrogen atom of a hydroxy group is substituted with an acid crosslinking reactive group or an acid dissociation reactive group; n¹ and n^{1′} are as defined as n¹ in the above formula (2B); p and p′ are as defined as p in the above formula (2B); and members in at least one combination selected from R^{1B} and R^{1B′}, n¹ and n^{1′}, p and p′, and the substitution position of R^{1B} and the substitution position of R^{1B′} differ from each other.

<13> The optical component forming composition according to <11>, wherein the tellurium-containing compound is represented by the following formula (3B):

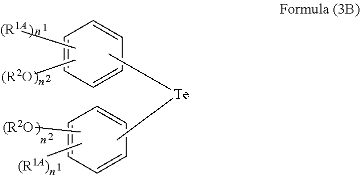

wherein R^{1A}, R², n¹, and n² are as defined in the above formula (2B).

<14> The optical component forming composition according to <13>, wherein the tellurium-containing compound is represented by the following formula (4B):

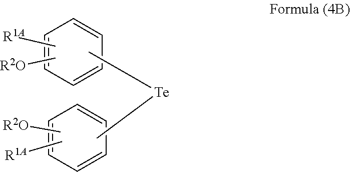

wherein R¹, R², and X¹ are as defined in the above formula (3B).

<15> The optical component forming composition according to any one of <5> to <7>, <9> to <11>, <13> to <14>, wherein the tellurium-containing compound has at least one acid dissociation reactive group as the R².
<16> The optical component forming composition according to any one of <5> to <7>, <9> to <11>, <13> to <14>, wherein all of the R² in the tellurium-containing compound are hydrogen atoms.
<17> The optical component forming composition according to <1>, wherein the tellurium-containing resin is a resin comprising a constitutional unit derived from a compound represented by the following formula (A-1):

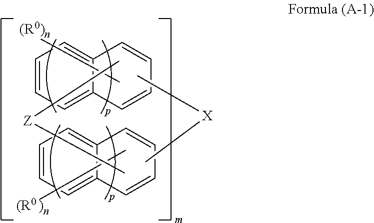

wherein X is a 2m-valent group of 0 to 60 carbon atoms containing tellurium; Z is an oxygen atom, a sulfur atom, or non-crosslinked state; each R⁰ is independently selected from the group consisting of a monovalent group containing an oxygen atom, a monovalent group containing a sulfur atom, a monovalent group containing a nitrogen atom, a hydrocarbon group, a halogen atom, and a combination thereof; m is an integer of 1 to 4; each p is independently an integer of 0 to 2; and each n is independently an integer of 0 to (5+2×p).

<18> The optical component forming composition according to <1>, wherein the tellurium-containing resin is a resin comprising a constitutional unit derived from a compound represented by the following formula (A-2):

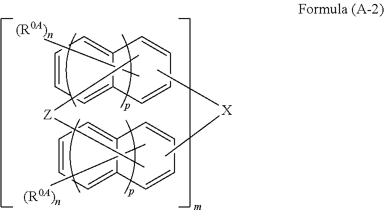

wherein X is a 2m-valent group of 0 to 60 carbon atoms containing tellurium; Z is an oxygen atom, a sulfur atom, a single bond, or non-crosslinked state; each R^{0A} is independently selected from the group consisting of a hydrocarbon group, a halogen atom, a cyano group, a nitro group, an amino group, an alkyl group of 1 to 30 carbon atoms, an alkenyl group of 2 to 30 carbon atoms, an aryl group of 6 to 40 carbon atoms, a hydroxy group or a group in which a hydrogen atom of a hydroxy group is substituted with an acid crosslinking reactive group or an acid dissociation reactive group, and a combination thereof, wherein the alkyl group, the alkenyl group, and the aryl group each optionally have an ether bond, a ketone bond, or an ester bond; m is an integer of 1 to 4; each p is independently an integer of 0 to 2; and each n is independently an integer of 0 to (5+2×p).

<19> The optical component forming composition according to <1>, wherein the tellurium-containing resin is a resin comprising a constitutional unit derived from a compound represented by the following formula (A-3):

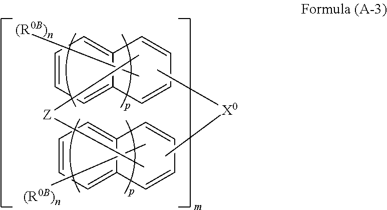

wherein X⁰ is a 2m-valent group of 0 to 30 carbon atoms containing tellurium; Z is an oxygen atom, a sulfur atom, or non-crosslinked state; each R^{0B} is independently a monovalent group containing an oxygen atom, a monovalent group containing a sulfur atom, a monovalent group containing a nitrogen atom, a hydrocarbon group, or a halogen atom; m is an integer of 1 to 4; each p is independently an integer of 0 to 2; and each n is independently an integer of 0 to (5+2×p).

<20> The optical component forming composition according to <1>, wherein the tellurium-containing resin is a resin comprising a constitutional unit represented by the following formula (B1-M):

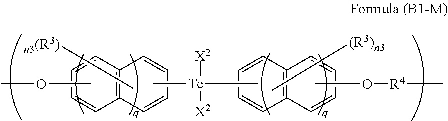

wherein each X² is independently a monovalent group containing an oxygen atom, a monovalent group containing a sulfur atom, a monovalent group containing a nitrogen atom, a hydrocarbon group, a hydrogen atom, or a halogen atom; each R³ is independently a monovalent group containing an oxygen atom, a monovalent group containing a sulfur atom, a monovalent group containing a nitrogen atom, a hydrocarbon group, or a halogen atom; q is an integer of 0 to 2; n³ is an integer of 0 to (4+2×q); and R⁴ is a single bond or any structure represented by the following general formula (5):

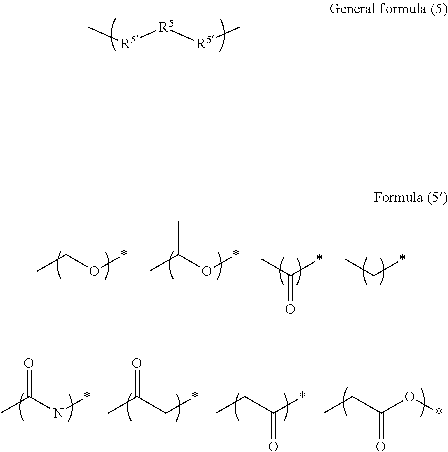

wherein R⁵ is a substituted or unsubstituted linear alkylene group of 1 to 20 carbon atoms, branched alkylene group of 3 to 20 carbon atoms, or cyclic alkylene group of 3 to 20 carbon atoms, or a substituted or unsubstituted arylene group of 6 to 20 carbon atoms; and each R^{5′} is independently any structure of the above formula (5′) wherein * indicates that this portion is connected to R⁵.

<21> The optical component forming composition according to <20>, wherein the R⁴ in the tellurium-containing resin is any structure represented by the above general formula (5).
<22> The optical component forming composition according to <20>, wherein the tellurium-containing resin is a resin comprising a constitutional unit represented by the following formula (B2-M′):

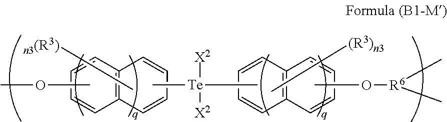

wherein X², R³, q, and n³ are as defined in the formula (B1-M); and R⁶ is any structure represented by the following general formula (6):

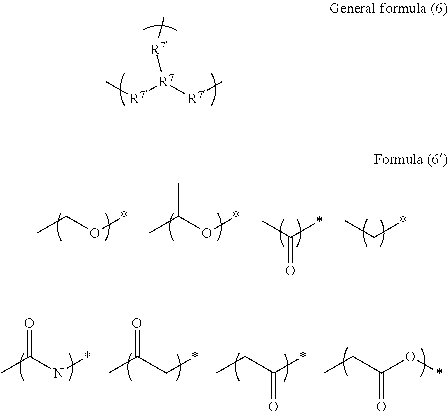

wherein R⁷ is a substituted or unsubstituted linear alkylene group of 1 to 20 carbon atoms, branched alkylene group of 3 to 20 carbon atoms, or cyclic alkylene group of 3 to 20 carbon atoms, or a substituted or unsubstituted arylene group of 6 to 20 carbon atoms; each R^{7′} is independently any structure of the above formula (6′) wherein * indicates that this portion is connected to R⁷.

<23> The optical component forming composition according to <1>, wherein the tellurium-containing resin is a resin comprising a constitutional unit represented by the following formula (C1):

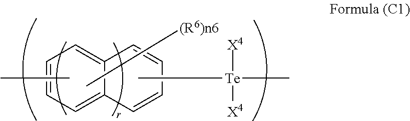

wherein each X⁴ is independently a monovalent group containing an oxygen atom, a monovalent group containing a sulfur atom, a monovalent group containing a nitrogen atom, a hydrocarbon group, a hydrogen atom, or a halogen atom; each R⁶ is independently a monovalent group containing an oxygen atom, a monovalent group containing a sulfur atom, a monovalent group containing a nitrogen atom, a hydrocarbon group, or a halogen atom; r is an integer of 0 to 2; and n⁶ is an integer of 2 to (4+2×r).

<24> The optical component forming composition according to <1>, wherein the tellurium-containing resin is a resin comprising a constitutional unit represented by the following formula (B3-M):

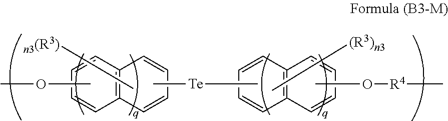

wherein each R³ is independently a monovalent group containing an oxygen atom, a monovalent group containing a sulfur atom, a monovalent group containing a nitrogen atom, a hydrocarbon group, or a halogen atom; q is an integer of 0 to 2; n³ is an integer of 0 to (4+2×q); and R⁴ is a single bond or any structure represented by the following general formula (5):

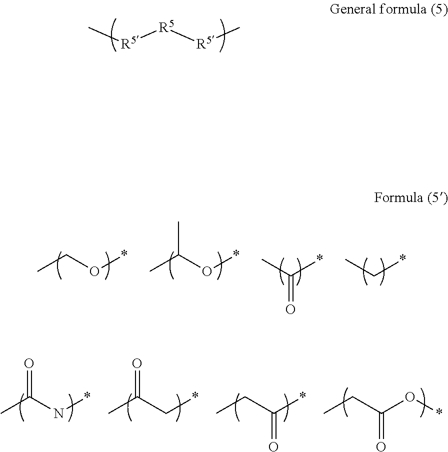

wherein R⁵ is a substituted or unsubstituted linear alkylene group of 1 to 20 carbon atoms, branched alkylene group of 3 to 20 carbon atoms, or cyclic alkylene group of 3 to 20 carbon atoms, or a substituted or unsubstituted arylene group of 6 to 20 carbon atoms; each R^{5′} is independently any structure of the above formula (5′) wherein * indicates that this portion is connected to R⁵ wherein * indicates that this portion is connected to R⁵.

<25> The optical component forming composition according to <24>, wherein the R⁴ in the tellurium-containing resin is any structure represented by the above general formula (5).
<26> The optical component forming composition according to <24>, wherein the tellurium-containing resin is a resin comprising a constitutional unit represented by the following formula (B4-M′):

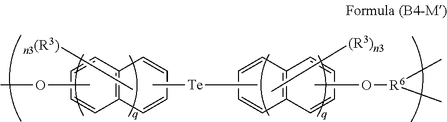

wherein R³, q, and n³ are as defined in the formula (B3-M); and R⁶ is any structure represented by the following general formula (6):

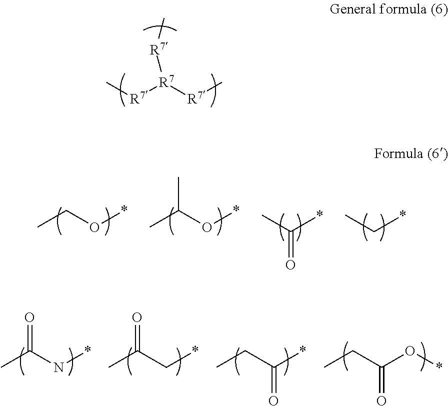

wherein R⁷ is a substituted or unsubstituted linear alkylene group of 1 to 20 carbon atoms, branched alkylene group of 3 to 20 carbon atoms, or cyclic alkylene group of 3 to 20 carbon atoms, or a substituted or unsubstituted arylene group of 6 to 20 carbon atoms; and each R^{7′} is independently any structure of the above formula (6′) wherein * indicates that this portion is connected to R⁷.

<27> The optical component forming composition according to <1>, wherein the tellurium-containing resin is a resin comprising a constitutional unit represented by the following formula (C2):

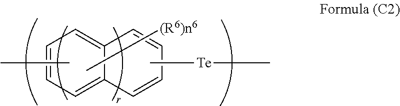

wherein each R⁶ is independently a monovalent group containing an oxygen atom, a monovalent group containing a sulfur atom, a monovalent group containing a nitrogen atom, a hydrocarbon group, or a halogen atom; r is an integer of 0 to 2; and n⁶ is an integer of 2 to (4+2×r).

<28> A method for producing the optical component forming composition according to any one of <1> to <27>, comprising the step of reacting a tellurium halide with a substituted or unsubstituted phenol derivative in the presence of a basic catalyst to synthesize the tellurium-containing compound.
<29> The optical component forming composition according to any one of <1> to <27>, further comprising a solvent.
<30> The optical component forming composition according to <29>, further comprising an acid generating agent.
<31> The optical component forming composition according to <29> or <30>, further comprising an acid crosslinking agent.
<32> A cured product obtained using the optical component forming composition according to any one of <1> to <31>.

### Advantageous Effects of Invention

The present invention can provide an optical component forming composition which is usefully used in optical materials, and a cured product thereof.

### DESCRIPTION OF EMBODIMENTS

Hereinafter, an embodiment of the present invention will be described (hereinafter, also referred to as “present embodiment”). The present embodiment is given in order to illustrate the present invention. The present invention is not limited to only the present embodiment.

### [Optical Component Forming Composition and Cured Product Thereof]

The optical component forming composition of the present embodiment is an optical component forming composition containing a tellurium-containing compound or resin. By containing the tellurium-containing compound or resin, the optical component forming composition of the present embodiment can be expected to have high refractive index and high transparency and is further expected to have storage stability, the ability to form a structure (the ability to form a film), and heat resistance. The optical component forming composition contains one or more selected from, for example, a compound represented by the formula (A-1) mentioned later and a resin obtained using this compound as a monomer (i.e., comprising a constitutional unit derived from the compound represented by the formula (A-1)).

The cured product of the present invention obtained by curing the optical component forming composition is prevented from being stained by heat treatment in a wide range from a low temperature to a high temperature and can be expected to have high refractive index and high transparency.

### (Tellurium-Containing Compound Represented by Formula (A-1))

According to the first embodiment, the optical component forming composition of the present embodiment can contain a tellurium-containing compound represented by the following formula (A-1):

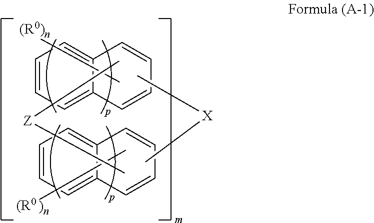

(In the formula (A-1), X is a 2m-valent group of 0 to 60 carbon atoms containing tellurium; Z is an oxygen atom, a sulfur atom, or non-crosslinked state; each R⁰ is independently selected from the group consisting of a monovalent group containing an oxygen atom, a monovalent group containing a sulfur atom, a monovalent group containing a nitrogen atom, a hydrocarbon group, a halogen atom, and a combination thereof; m is an integer of 1 to 4; each p is independently an integer of 0 to 2; and each n is independently an integer of 0 to (5+2×p).)

The chemical structure of the compound contained in the optical component forming composition of the present embodiment can be determined by ¹H-NMR analysis.

The compound contained in the optical component forming composition of the present embodiment contains tellurium as shown in the above formula (A-1) and therefore has high refractive index and high transparency. The compound has a benzene skeleton or a naphthalene skeleton or the like and is therefore excellent in heat resistance. Furthermore, the compound is stable against heat treatment in a wide range from a low temperature to a high temperature and is prevented from being stained by such heat treatment. Hence, the compound is also useful as various optical component forming compositions. Moreover, the compound has the structure of the above formula (A-1) and is therefore excellent in storage stability and the ability to form a structure (the ability to form a film).

An optical component to which the cured product of the optical component forming composition of the present embodiment can be applied is used in a film form or a sheet form and is also useful as a plastic lens (prism lens, lenticular lens, microlens, Fresnel lens, viewing angle control lens, contrast improving lens, etc.), a phase difference film, a film for electromagnetic wave shielding, a prism, an optical fiber, a solder resist for flexible printed wiring, a plating resist, an interlayer insulating film for multilayer printed circuit boards, and a photosensitive optical waveguide.

In the above formula (A-1), m is an integer of 1 to 4. When m is an integer of 2 or larger, the structural formulae of m repeat units may be the same or different. In the above formula (A-1), m is preferably 1 to 3 from the viewpoint of resist properties such as heat resistance, resolution, and roughness.

Although the compound of the present embodiment is not a polymer, the structure indicated within the parentheses [ ] bonded to X in the above formula (A-1) is referred to as “structural formula of a repeat unit” (the same holds true for formulae given below) for the sake of convenience.

In the above formula (A-1), each p is independently an integer of 0 to 2 and is a value that determines the structure of the accompanying ring structure (a ring structure represented by naphthalene in the formula (A-1) (hereinafter, the ring structure is also simply referred to as “ring structure A”)). Specifically, as shown below, in the formula (A-1), the ring structure A refers to a benzene structure (p=0), a naphthalene structure (p=1), or a tricyclic structure such as anthracene or phenanthrene (p=2). The ring structure A is not particularly limited, but is preferably a benzene structure or a naphthalene structure from the viewpoint of solubility. In the formula (A-1), X, Z, and R⁰ are bonded to any possible site on the ring structure A.

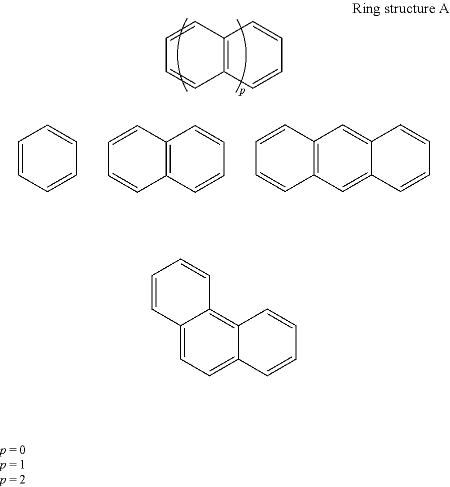

In the above formula (A-1), X is a 2m-valent group of 0 to 60 carbon atoms containing tellurium. X is a single bond containing tellurium or a 2m-valent hydrocarbon group of 0 to 60 carbon atoms containing tellurium.

The 2m-valent group refers to an alkylene group of 1 to 60 carbon atoms (m=1), an alkanetetrayl group of 1 to 60 carbon atoms (m=2), an alkanehexayl group of 2 to 60 carbon atoms (m=3), or an alkaneoctayl group of 3 to 60 carbon atoms (m=4). Examples of the 2m-valent group include groups having a linear, branched, or cyclic structure.

Also, the 2m-valent hydrocarbon group may have an alicyclic hydrocarbon group, a double bond, a hetero atom, or an aromatic group of 6 to 60 carbon atoms. Herein, the alicyclic hydrocarbon group also includes bridged alicyclic hydrocarbon groups.

X preferably has a condensed polycyclic aromatic group (particularly, a bicyclic to tetracyclic condensed ring structure) from the viewpoint of heat resistance and preferably has a polyphenyl group such as a biphenyl group from the viewpoint of solubility in a safe solvent and heat resistance.

Specific examples of the 2m-valent group of 0 to 60 carbon atoms containing tellurium, represented by X include the following groups:

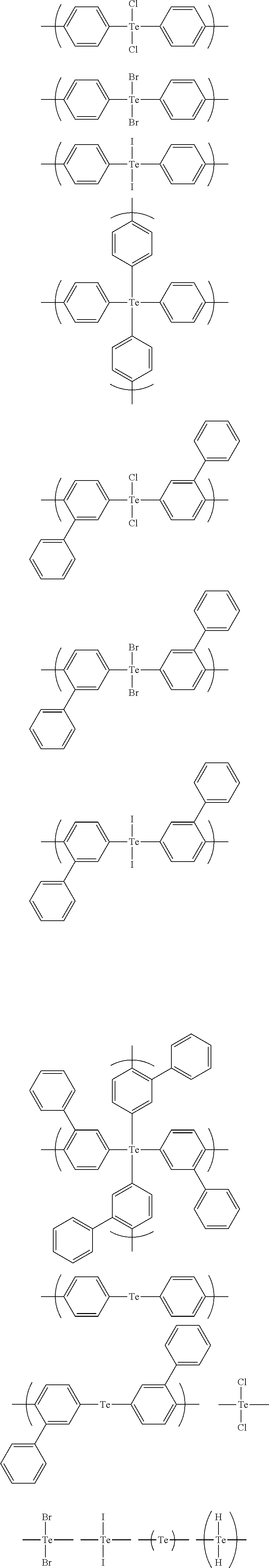

In the above formula (A-1), Z is an oxygen atom, a sulfur atom, or non-crosslinked state. When m is 2 or larger, Z may be the same or different. Also, when m is 2 or larger, the structural formulae of different repeat units may be bonded via Z. For example, when m is 2 or larger, the structural formulae of different repeat units may be bonded via Z and the structural formulae of a plurality of repeat units may constitute a cup like structure or the like. Z is not particularly limited, but is preferably an oxygen atom or a sulfur atom from the viewpoint of heat resistance.

In the above formula (A-1), R⁰ is a monovalent group containing an oxygen atom, a monovalent group containing a sulfur atom, a monovalent group containing a nitrogen atom, a halogen atom, or a combination thereof.

Herein, examples of the monovalent group containing an oxygen atom include, but not limited to, acyl groups of 1 to 20 carbon atoms, alkoxycarbonyl groups of 2 to 20 carbon atoms, linear alkyloxy groups of 1 to 6 carbon atoms, branched alkyloxy groups of 3 to 20 carbon atoms, cyclic alkyloxy groups of 3 to 20 carbon atoms, linear alkenyloxy groups of 2 to 6 carbon atoms, branched alkenyloxy groups of 3 to 6 carbon atoms, cyclic alkenyloxy groups of 3 to 10 carbon atoms, aryloxy groups of 6 to 10 carbon atoms, acyloxy groups of 1 to 20 carbon atoms, alkoxycarbonyloxy groups of 2 to 20 carbon atoms, alkoxycarbonylalkyl groups of 2 to 20 carbon atoms, 1-substituted alkoxymethyl groups of 2 to 20 carbon atoms, cyclic ether oxy groups of 2 to 20 carbon atoms, alkoxyalkyloxy groups of 2 to 20 carbon atoms, a glycidyloxy group, allyloxy groups, (meth)acryl groups, a glycidyl acrylate group, a glycidyl methacrylate group, and a hydroxy group.

Examples of the acyl groups of 1 to 20 carbon atoms include, but not limited to, a methanoyl group (a formyl group), an ethanoyl group (an acetyl group), a propanoyl group, a butanoyl group, a pentanoyl group, a hexanoyl group, an octanoyl group, a decanoyl group, and a benzoyl group.

Examples of the alkoxycarbonyl groups of 2 to 20 carbon atoms include, but not limited to, a methoxycarbonyl group, an ethoxycarbonyl group, a propoxycarbonyl group, a butoxycarbonyl group, a pentyloxycarbonyl group, a hexyloxycarbonyl group, an octyloxycarbonyl group, and a decyloxycarbonyl group.

Examples of the linear alkyloxy groups of 1 to 6 carbon atoms include, but not limited to, a methoxy group, an ethoxy group, a n-propoxy group, a n-butoxy group, a n-pentyloxy group, and a n-hexyloxy group.

Examples of the branched alkyloxy groups of 3 to 20 carbon atoms include, but not limited to, an isopropoxy group, an isobutoxy group, and a tert-butoxy group.

Examples of the cyclic alkyloxy groups of 3 to 20 carbon atoms include, but not limited to, a cyclopropoxy group, a cyclobutoxy group, a cyclopentyloxy group, a cyclohexyloxy group, a cyclooctyloxy group, and a cyclodecyloxy group.

Examples of the linear alkenyloxy groups of 2 to 6 carbon atoms include, but not limited to, a vinyloxy group, a 1-propenyloxy group, a 2-propenyloxy group, a 1-butenyloxy group, and a 2-butenyloxy group.

Examples of the branched alkenyloxy groups of 3 to 6 carbon atoms include, but not limited to, an isopropenyloxy group, an isobutenyloxy group, an isopentenyloxy group, and an isohexenyloxy group.

Examples of the cyclic alkenyloxy groups of 3 to 10 carbon atoms include, but not limited to, a cyclopropenyloxy group, a cyclobutenyloxy group, a cyclopentenyloxy group, a cyclohexenyloxy group, a cyclooctenyloxy group, and a cyclodecynyloxy group.

Examples of the aryloxy groups of 6 to 10 carbon atoms include, but not limited to, a phenyloxy group (a phenoxy group), a 1-naphthyloxy group, and a 2-naphthyloxy group.

Examples of the acyloxy groups of 1 to 20 carbon atoms include, but not limited to, a formyloxy group, an acetyloxy group, a propionyloxy group, a butyryloxy group, an isobutyryloxy group, and a benzoyloxy group.

Examples of the alkoxycarbonyloxy groups of 2 to 20 carbon atoms include, but not limited to, a methoxycarbonyloxy group, an ethoxycarbonyloxy group, a propoxycarbonyloxy group, a butoxycarbonyloxy group, an octyloxycarbonyloxy group, and a decyloxycarbonyloxy group.

Examples of the alkoxycarbonylalkyl groups of 2 to 20 carbon atoms include, but not limited to, a methoxycarbonylmethyl group, an ethoxycarbonylmethyl group, a n-propoxycarbonylmethyl group, an isopropoxycarbonylmethyl group, and a n-butoxycarbonylmethyl group.

Examples of the 1-substituted alkoxymethyl groups of 2 to 20 carbon atoms include, but not limited to, a 1-cyclopentylmethoxymethyl group, a 1-cyclopentylethoxymethyl group, a 1-cyclohexylmethoxymethyl group, a 1-cyclohexylethoxymethyl group, a 1-cyclooctylmethoxymethyl group, and a 1-adamantylmethoxymethyl group.

Examples of the cyclic ether oxy groups of 2 to 20 carbon atoms include, but not limited to, a tetrahydropyranyloxy group, a tetrahydrofuranyloxy group, a tetrahydrothiopyranyloxy group, a tetrahydrothiofuranyloxy group, a 4-methoxytetrahydropyranyloxy group, and a 4-methoxytetrahydrothiopyranyloxy group.

Examples of the alkoxyalkyloxy groups of 2 to 20 carbon atoms include, but not limited to, a methoxymethoxy group, an ethoxyethoxy group, a cyclohexyloxymethoxy group, a cyclohexyloxyethoxy group, a phenoxymethoxy group, and a phenoxyethoxy group.

Examples of the (meth)acryl groups include, but not limited to, an acryloyloxy group and a methacryloyloxy group. The glycidyl acrylate group is not particularly limited as long as it can be obtained by reacting a glycidyloxy group with acrylic acid. The glycidyl methacrylate group is not particularly limited as long as it can be obtained by reacting a glycidyloxy group with methacrylic acid.

Examples of the monovalent group containing a sulfur atom include, but not limited to, a thiol group. The monovalent group containing a sulfur atom is preferably a group in which a sulfur atom is directly bonded to a carbon atom constituting the ring structure (A-1) in the formula (A-1).

Examples of the monovalent group containing a nitrogen atom include, but not limited to, a nitro group, an amino group, and a diazo group. The monovalent group containing a nitrogen atom is preferably a group in which a nitrogen atom is directly bonded to a carbon atom constituting the ring structure (A-1) in the formula (A-1).

Examples of the hydrocarbon group include, but not limited to, linear alkyl groups of 1 to 6 carbon atoms, branched alkyl groups of 3 to 6 carbon atoms, cyclic alkyl groups of 3 to 10 carbon atoms, linear alkenyl groups of 2 to 6 carbon atoms, branched alkenyl groups of 3 to 6 carbon atoms, cyclic alkenyl groups of 3 to 10 carbon atoms, and aryl groups of 6 to 10 carbon atoms.

Examples of the linear alkyl groups of 1 to 6 carbon atoms include, but not limited to, a methyl group, an ethyl group, a n-propyl group, a n-butyl group, a n-pentyl group, and a n-hexyl group.

Examples of the branched alkyl groups of 3 to 6 carbon atoms include, but not limited to, an isopropyl group, an isobutyl group, a tert-butyl group, a neopentyl group, and a 2-hexyl group.

Examples of the cyclic alkyl groups of 3 to 10 carbon atoms include, but not limited to, a cyclopropyl group, a cyclobutyl group, a cyclopentyl group, a cyclohexyl group, a cyclooctyl group, and a cyclodecyl group.

Examples of the linear alkenyl groups of 2 to 6 carbon atoms include, but not limited to, a vinyl group, a 1-propenyl group, a 2-propenyl group (an allyl group), a 1-butenyl group, a 2-butenyl group, a 2-pentenyl group, and a 2-hexenyl group.

Examples of the branched alkenyl groups of 3 to 6 carbon atoms include, but not limited to, an isopropenyl group, an isobutenyl group, an isopentenyl group, and an isohexenyl group.

Examples of the cyclic alkenyl groups of 3 to 10 carbon atoms include, but not limited to, a cyclopropenyl group, a cyclobutenyl group, a cyclopentenyl group, a cyclohexenyl group, a cyclohexenyl group, a cyclooctenyl group, and a cyclodecynyl group.

Examples of the aryl groups of 6 to 10 carbon atoms include, but not limited to, a phenyl group and a naphthyl group.

Examples of the halogen atom include, but not limited to, a fluorine atom, a chlorine atom, a bromine atom, and an iodine atom.

In the above formula (A-1), each n is independently an integer of 0 to (5+2×p). In the present embodiment, at least one n in the above formula (A-1) is preferably an integer of 1 to 4 from the viewpoint of solubility in a solvent.

In the present embodiment, at least one R⁰ in the above formula (A-1) is preferably a monovalent group containing an oxygen atom from the viewpoint of solubility in a solvent and the introduction of crosslinkability.

The tellurium-containing compound represented by the above formula (A-1) is preferably a tellurium-containing compound represented by the following formula (A-2) from the viewpoint of curability:

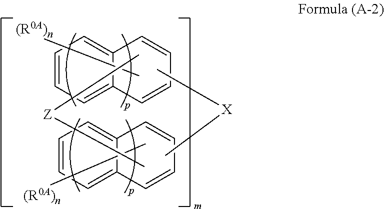

(In the formula (A-2), X is a 2m-valent group of 0 to 60 carbon atoms containing tellurium; Z is an oxygen atom, a sulfur atom, a single bond, or non-crosslinked state; each R^{0A} is independently selected from the group consisting of a hydrocarbon group, a halogen atom, a cyano group, a nitro group, an amino group, an alkyl group of 1 to 30 carbon atoms, an alkenyl group of 2 to 30 carbon atoms, an aryl group of 6 to 40 carbon atoms, a hydroxy group or a group in which a hydrogen atom of a hydroxy group is substituted with an acid crosslinking reactive group or an acid dissociation reactive group, and a combination thereof, wherein the alkyl group, the alkenyl group, and the aryl group each optionally have an ether bond, a ketone bond, or an ester bond; m is an integer of 1 to 4; each p is independently an integer of 0 to 2; and each n is independently an integer of 0 to (5+2×p).)

The “acid crosslinking reactive group” and the “acid dissociation reactive group” in R^{0A} will be mentioned later.

The tellurium-containing compound represented by the above formula (A-1) is preferably a tellurium-containing compound represented by the following formula (A-3) from the viewpoint of solubility in a safe solvent:

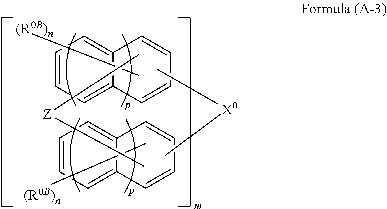

(In the formula (A-3), X⁰ is a 2m-valent group of 0 to 30 carbon atoms containing tellurium; Z is an oxygen atom, a sulfur atom, or non-crosslinked state; each R^{0B} is independently a monovalent group containing an oxygen atom, a monovalent group containing a sulfur atom, a monovalent group containing a nitrogen atom, a hydrocarbon group, or a halogen atom; m is an integer of 1 to 4; each p is independently an integer of 0 to 2; and each n is independently an integer of 0 to (5+2×p).)

In the present embodiment, the tellurium-containing compound represented by the above formula (A-1) is preferably a compound other than BMPT, BHPT, and TDP mentioned later from the viewpoint of the pattern shape of the resulting resist.

### —Tellurium-Containing Compound Represented by Formula (1A)—

The tellurium-containing compound represented by the above formula (A-1) is preferably a tellurium-containing compound represented by the following formula (1A):

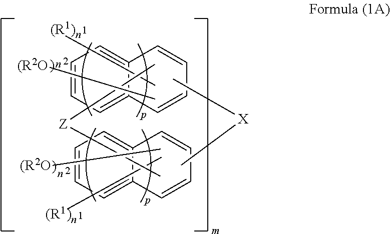

(In the formula (1A), X, Z, m, and p are as defined in the above formula (A-1); each R¹ is independently selected from the group consisting of a hydrocarbon group, a halogen atom, a cyano group, a nitro group, an amino group, an alkyl group of 1 to 30 carbon atoms, an alkenyl group of 2 to 30 carbon atoms, an aryl group of 6 to 40 carbon atoms, and a combination thereof, wherein the alkyl group, the alkenyl group, and the aryl group each optionally have an ether bond, a ketone bond, or an ester bond; each R² is independently a hydrogen atom, an acid crosslinking reactive group, or an acid dissociation reactive group; each n¹ is independently an integer of 0 to (5+2×p); and each n² is independently an integer of 0 to (5+2×p), provided that at least one n² is an integer of 1 to (5+2×p).)

In the formula (1A), each n¹ is independently an integer of 0 to (5+2×p), and each n² is independently an integer of 0 to (5+2×p). At least one n² is an integer of 1 to (5+2×p). Specifically, the tellurium-containing compound of the general formula (1A has at least one “—OR²” per ring structure A. In the formula (1), X, Z, R¹, and —OR² are bonded to any possible site on the ring structure A. Therefore, the upper limit of n¹+n² in one ring structure A corresponds to the upper limit of the number of possible bonding sites on the ring structure A also taking X and Z and the bonding sites into consideration.

Each R¹ is independently selected from the group consisting of a hydrocarbon group, a halogen atom, a cyano group, a nitro group, an amino group, an alkyl group of 1 to 30 carbon atoms, an alkenyl group of 2 to 30 carbon atoms, an aryl group of 6 to 40 carbon atoms, and a combination thereof. Herein, the alkyl group, the alkenyl group, and the aryl group each optionally have an ether bond, a ketone bond, or an ester bond.

As mentioned above, the hydrocarbon group represented by R¹ is a substituted or unsubstituted linear, substituted or unsubstituted branched, or substituted or unsubstituted cyclic hydrocarbon group.

Examples of the linear, branched, or cyclic hydrocarbon group include, but not limited to, linear alkyl groups of 1 to 30 carbon atoms, branched alkyl groups of 3 to 30 carbon atoms, and cyclic alkyl groups of 3 to 30 carbon atoms.

Examples of the linear alkyl groups of 1 to 30 carbon atoms include, but not limited to, a methyl group, an ethyl group, a n-propyl group, a n-butyl group, a n-pentyl group, and a n-hexyl group.

Examples of the branched alkyl groups of 3 to 30 carbon atoms include, but not limited to, an isopropyl group, an isobutyl group, a tert-butyl group, a neopentyl group, and a 2-hexyl group.

Examples of the cyclic alkyl groups of 3 to 30 carbon atoms include, but not limited to, a cyclopropyl group, a cyclobutyl group, a cyclopentyl group, a cyclohexyl group, a cyclooctyl group, and a cyclodecyl group.

As mentioned above, the aryl group represented by R¹ is, but not limited to, an aryl group of 6 to 40 carbon atoms. Examples thereof include a phenyl group and a naphthyl group.

As mentioned above, the alkenyl group represented by R¹ is, but not limited to a substituted or unsubstituted alkenyl group. Examples thereof include linear alkenyl groups of 2 to 30 carbon atoms, branched alkenyl groups of 3 to 30 carbon atoms, and cyclic alkenyl groups of 3 to 30 carbon atoms.

Examples of the linear alkenyl groups of 2 to 30 carbon atoms include, but not limited to, a vinyl group, a 1-propenyl group, a 2-propenyl group (an allyl group), a 1-butenyl group, a 2-butenyl group, a 2-pentenyl group, and a 2-hexenyl group.

Examples of the branched alkenyl groups of 3 to 30 carbon atoms include, but not limited to, an isopropenyl group, an isobutenyl group, an isopentenyl group, and an isohexenyl group.

Examples of the cyclic alkenyl groups of 3 to 30 carbon atoms include, but not limited to, a cyclopropenyl group, a cyclobutenyl group, a cyclopentenyl group, a cyclohexenyl group, a cyclohexenyl group, a cyclooctenyl group, and a cyclodecynyl group.

Examples of the halogen atom include a fluorine atom, a chlorine atom, a bromine atom, and an iodine atom.

In the present specification, the term “substituted” means that one or more hydrogen atoms in a functional group are substituted with a halogen atom, a hydroxy group, a cyano group, a nitro group, a heterocyclic group, a linear aliphatic hydrocarbon group of 1 to 20 carbon atoms, a branched aliphatic hydrocarbon group of 3 to 20 carbon atoms, a cyclic aliphatic hydrocarbon group of 3 to 20 carbon atoms, an aryl group of 6 to 20 carbon atoms, an aralkyl group of 7 to 30 carbon atoms, an alkoxy group of 1 to 20 carbon atoms, an amino group of 0 to 20 carbon atoms, an alkenyl group of 2 to 20 carbon atoms, an acyl group of 1 to 20 carbon atoms, an alkoxycarbonyl group of 2 to 20 carbon atoms, an alkyloyloxy group of 1 to 20 carbon atoms, an aryloyloxy group of 7 to 30 carbon atoms, or an alkylsilyl group of 1 to 20 carbon atoms, unless otherwise defined.

Examples of the unsubstituted linear aliphatic hydrocarbon group of 1 to 20 carbon atoms include a methyl group, an ethyl group, a propyl group, a butyl group, a pentyl group, a hexyl group, an octyl group, a decyl group, a dodecyl group, a hexadecyl group, and an octadecyl group.

Examples of the substituted linear aliphatic hydrocarbon group of 1 to 20 carbon atoms include a fluoromethyl group, a 2-hydroxyethyl group, a 3-cyanopropyl group, and a 20-nitrooctadecyl group.

Examples of the unsubstituted branched aliphatic hydrocarbon group of 3 to 20 carbon atoms include an isopropyl group, an isobutyl group, a tertiary butyl group, a neopentyl group, a 2-hexyl group, a 2-octyl group, a 2-decyl group, a 2-dodecyl group, a 2-hexadecyl group, and a 2-octadecyl group.

Examples of the substituted branched aliphatic hydrocarbon group of 3 to 20 carbon atoms include a 1-fluoroisopropyl group and a 1-hydroxy-2-octadecyl group.

Examples of the unsubstituted cyclic aliphatic hydrocarbon group of 3 to 20 carbon atoms include a cyclopropyl group, a cyclobutyl group, a cyclopentyl group, a cyclohexyl group, a cyclooctyl group, a cyclodecyl group, a cyclododecyl group, a cyclohexadecyl group, and a cyclooctadecyl group.

Examples of the substituted cyclic aliphatic hydrocarbon group of 3 to 20 carbon atoms include a 2-fluorocyclopropyl group and a 4-cyanocyclohexyl group.

Examples of the unsubstituted aryl group of 6 to 20 carbon atoms include a phenyl group and a naphthyl group.

Examples of the substituted aryl group of 6 to 20 carbon atoms include a 4-isopropylphenyl group, a 4-cyclohexylphenyl group, a 4-methylphenyl group, and a 6-fluoronaphthyl group.

Examples of the unsubstituted alkenyl group of 2 to 20 carbon atoms include a vinyl group, a propynyl group, a butynyl group, a pentynyl group, a hexynyl group, an octynyl group, a decynyl group, a dodecynyl group, a hexadecynyl group, and an octadecynyl group.

Examples of the substituted alkenyl group of 2 to 20 carbon atoms include a chloropropynyl group.

Examples of the halogen atom include a fluorine atom, a chlorine atom, a bromine atom, and an iodine atom.

In the formula (1A), each R² is independently a hydrogen atom, an acid crosslinking reactive group, or an acid dissociation reactive group.

In the present embodiment, the “acid crosslinking reactive group” refers to a characteristic group that reacts in the presence of a radical or an acid or an alkali and varies in solubility in an acid, an alkali, or an organic solvent for use in a coating solvent or a developing solution. Examples of the acid crosslinking reactive group include allyl groups, (meth)acryloyl groups, a vinyl group, an epoxy group, alkoxymethyl groups, and a cyanato group. The acid crosslinking reactive group is not limited thereto as long as it reacts in the presence of a radical or an acid or an alkali. The acid crosslinking reactive group preferably has the property of causing chained cleavage reaction in the presence of an acid, from the viewpoint of improvement in productivity.

In the present embodiment, the “acid dissociation reactive group” refers to a characteristic group that is cleaved in the presence of an acid to cause a change such as an alkali soluble group. Examples of the alkali soluble group include, but not particularly limited to, a phenolic hydroxy group, a carboxyl group, a sulfonic acid group, and a hexafluoroisopropanol group. A phenolic hydroxy group and a carboxyl group are preferable, and a phenolic hydroxy group is particularly preferable. The acid dissociation reactive group is not particularly limited, but can be arbitrarily selected for use from among, for example, those proposed in hydroxystyrene based resins, (meth)acrylic acid based resins, and the like for use in chemically amplified resist compositions for KrF or ArF.

Preferable examples of the acid dissociation reactive group include a group selected from the group consisting of a substituted methyl group, a 1-substituted ethyl group, a 1-substituted n-propyl group, a 1-branched alkyl group, a silyl group, an acyl group, a 1-substituted alkoxymethyl group, a cyclic ether group, an alkoxycarbonyl group, and an alkoxycarbonylalkyl group which have the property of being dissociated by an acid. It is preferable that the acid dissociation reactive group has no crosslinkable functional group.

The substituted methyl group is not particularly limited, but can be usually a substituted methyl group of 2 to 20 carbon atoms and is preferably a substituted methyl group of 4 to 18 carbon atoms and more preferably a substituted methyl group of 6 to 16 carbon atoms. Specific examples of the substituted methyl group can include, but not limited to, a methoxymethyl group, a methylthiomethyl group, an ethoxymethyl group, a n-propoxymethyl group, an isopropoxymethyl group, a n-butoxymethyl group, a t-butoxymethyl group, a 2-methylpropoxymethyl group, an ethylthiomethyl group, a methoxyethoxymethyl group, a phenyloxymethyl group, a 1-cyclopentyloxymethyl group, a 1-cyclohexyloxymethyl group, a benzylthiomethyl group, a phenacyl group, a 4-bromophenacyl group, a 4-methoxyphenacyl group, a piperonyl group, and a substituent group represented by the following formula (13-1). Specific examples of R² in the following formula (13-1) include, but not limited to, a methyl group, an ethyl group, an isopropyl group, a n-propyl group, a t-butyl group, and a n-butyl group.

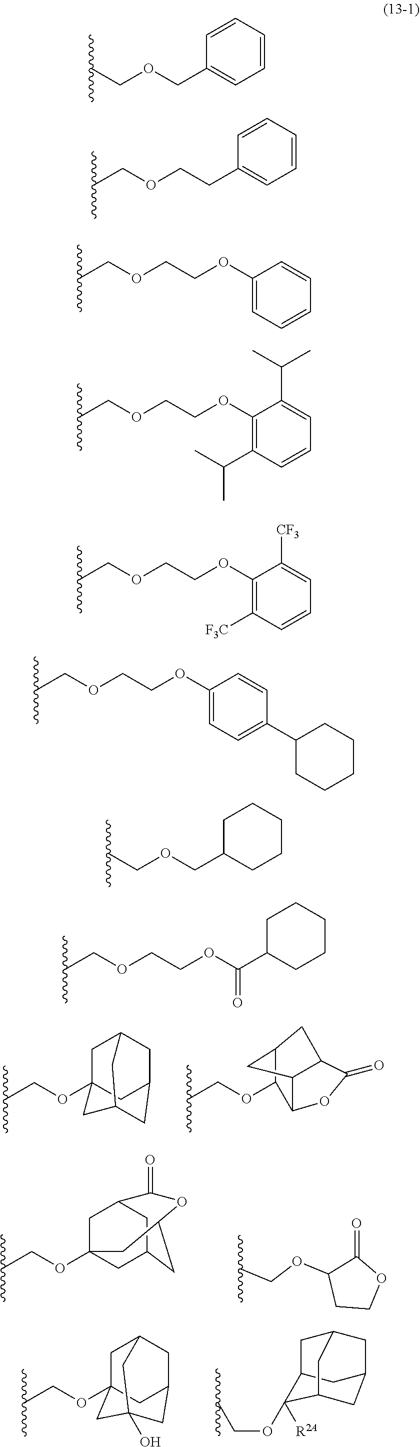

In the above formula (13-1), R^{2A} is an alkyl group of 1 to 4 carbon atoms.

The 1-substituted ethyl group is not particularly limited, but can be usually a 1-substituted ethyl group of 3 to 20 carbon atoms and is preferably a 1-substituted ethyl group of 5 to 18 carbon atoms and more preferably a substituted ethyl group of 7 to 16 carbon atoms. Specific examples of the 1-substituted ethyl group can include, but not limited to, a 1-methoxyethyl group, 1-methylthioethyl group, a 1,1-dimethoxyethyl group, a 1-ethoxyethyl group, a 1-ethylthioethyl group, a 1,1-diethoxyethyl group, a n-propoxyethyl group, an isopropoxyethyl group, a n-butoxyethyl group, a t-butoxyethyl group, a 2-methylpropoxyethyl group, a 1-phenoxyethyl group, a 1-phenylthioethyl group, a 1,1-diphenoxyethyl group, a 1-cyclopentyloxyethyl group, a 1-cyclohexyloxyethyl group, a 1-phenylethyl group, a 1,1-diphenylethyl group, and a substituent group represented by the following formula (13-2):

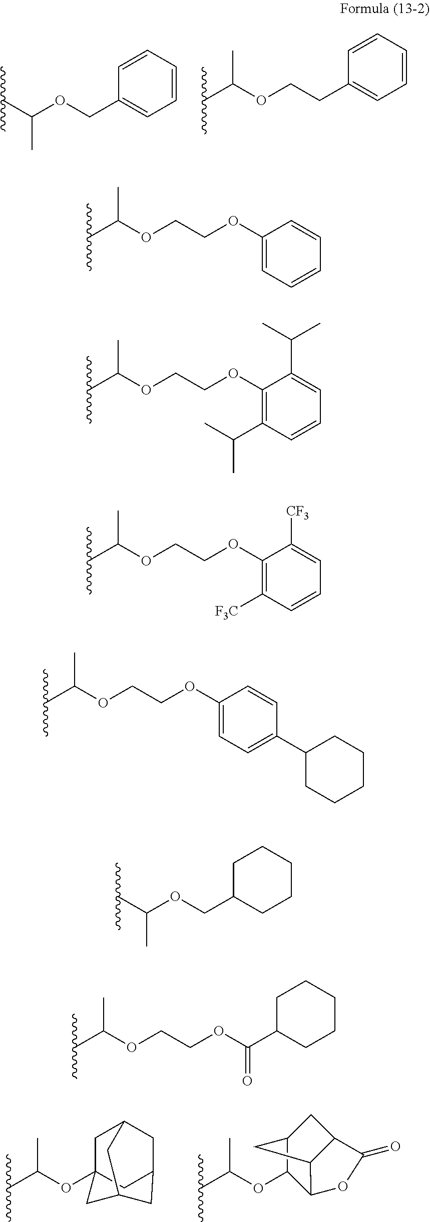

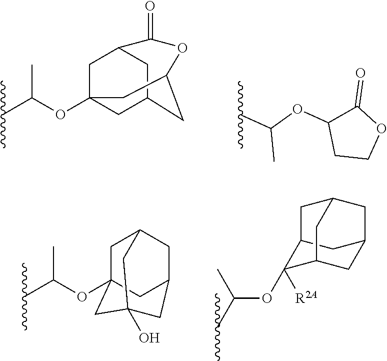

In the above formula (13-2), R^{2A} is as defined in the above formula (13-1).

The 1-substituted n-propyl group is not particularly limited, but can be usually a 1-substituted n-propyl group of 4 to 20 carbon atoms and is preferably a 1-substituted n-propyl group of 6 to 18 carbon atoms and more preferably a 1-substituted n-propyl group of 8 to 16 carbon atoms. Specific examples of the 1-substituted n-propyl group can include, but not limited to, a 1-methoxy-n-propyl group and a 1-ethoxy-n-propyl group.

The 1-branched alkyl group is not particularly limited, but can be usually a 1-branched alkyl group of 3 to 20 carbon atoms and is preferably a 1-branched alkyl group of 5 to 18 carbon atoms and more preferably a branched alkyl group of 7 to 16 carbon atoms. Specific examples of the 1-branched alkyl group can include, but not limited to, an isopropyl group, a sec-butyl group, a tert-butyl group, a 1,1-dimethylpropyl group, a 1-methylbutyl group, a 1,1-dimethylbutyl group, a 2-methyladamantyl group, and a 2-ethyladamantyl group.

The silyl group is not particularly limited, but can be usually a silyl group of 1 to 20 carbon atoms and is preferably a silyl group of 3 to 18 carbon atoms and more preferably a silyl group of 5 to 16 carbon atoms. Specific examples of the silyl group can include, but not limited to, a trimethylsilyl group, an ethyldimethylsilyl group, a methyldiethylsilyl group, a triethylsilyl group, a tert-butyldimethylsilyl group, a tert-butyldiethylsilyl group, a tert-butyldiphenylsilyl group, a tri-tert-butylsilyl group, and a triphenylsilyl group.

The acyl group is not particularly limited, but can be usually an acyl group of 2 to 20 carbon atoms and is preferably an acyl group of 4 to 18 carbon atoms and more preferably an acyl group of 6 to 16 carbon atoms. Specific examples of the acyl group can include, but not limited to, an acetyl group, a phenoxyacetyl group, a propionyl group, a butyryl group, a heptanoyl group, a hexanoyl group, a valeryl group, a pivaloyl group, an isovaleryl group, a lauroyl group, an adamantylcarbonyl group, a benzoyl group, and a naphthoyl group.

The 1-substituted alkoxymethyl group is not particularly limited, but can be usually a 1-substituted alkoxymethyl group of 2 to 20 carbon atoms and is preferably a 1-substituted alkoxymethyl group of 4 to 18 carbon atoms and more preferably a 1-substituted alkoxymethyl group of 6 to 16 carbon atoms. Specific examples of the 1-substituted alkoxymethyl group can include, but not limited to, a 1-cyclopentylmethoxymethyl group, a 1-cyclopentylethoxymethyl group, a 1-cyclohexylmethoxymethyl group, a 1-cyclohexylethoxymethyl group, a 1-cyclooctylmethoxymethyl group, and a 1-adamantylmethoxymethyl group.

The cyclic ether group is not particularly limited, but can be usually a cyclic ether group of 2 to 20 carbon atoms and is preferably a cyclic ether group of 4 to 18 carbon atoms and more preferably a cyclic ether group of 6 to 16 carbon atoms. Specific examples of the cyclic ether group can include, but not limited to, a tetrahydropyranyl group, a tetrahydrofuranyl group, a tetrahydrothiopyranyl group, a tetrahydrothiofuranyl group, a 4-methoxytetrahydropyranyl group, and a 4-methoxytetrahydrothiopyranyl group.

The alkoxycarbonyl group can be usually an alkoxycarbonyl group of 2 to 20 carbon atoms and is preferably an alkoxycarbonyl group of 4 to 18 carbon atoms and more preferably an alkoxycarbonyl group of 6 to 16 carbon atoms. Specific examples of the alkoxycarbonyl group can include, but not limited to, a methoxycarbonyl group, an ethoxycarbonyl group, a n-propoxycarbonyl group, an isopropoxycarbonyl group, a n-butoxycarbonyl group, a tert-butoxycarbonyl group, and a group of acid dissociation reactive groups represented by the following formula (13-3) wherein n=0.

The alkoxycarbonylalkyl group is not particularly limited, but can be usually an alkoxycarbonylalkyl group of 2 to 20 carbon atoms and is preferably an alkoxycarbonylalkyl group of 4 to 18 carbon atoms and more preferably an alkoxycarbonylalkyl group of 6 to 16 carbon atoms. Specific examples of the alkoxycarbonylalkyl group can include, but not limited to, a methoxycarbonylmethyl group, an ethoxycarbonylmethyl group, a n-propoxycarbonylmethyl group, an isopropoxycarbonylmethyl group, a n-butoxycarbonylmethyl group, and a group of acid dissociation reactive groups represented by the following formula (13-3) wherein n=1 to 4:

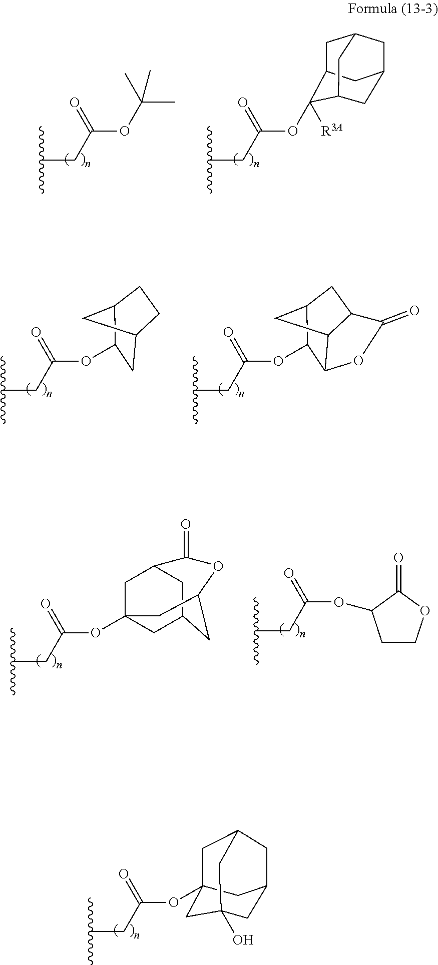

In the above formula (13-3), R^{3A} is a hydrogen atom or a linear or branched alkyl group of 1 to 4 carbon atoms; and n is an integer of 0 to 4.

Among these acid dissociation reactive groups, a substituted methyl group, a 1-substituted ethyl group, a 1-substituted alkoxymethyl group, a cyclic ether group, an alkoxycarbonyl group, and an alkoxycarbonylalkyl group are preferable. From the viewpoint of exerting higher sensitivity, a substituted methyl group, a 1-substituted ethyl group, an alkoxycarbonyl group, and an alkoxycarbonylalkyl group are more preferable, and an acid dissociation reactive group having a structure selected from a cycloalkane of 3 to 12 carbon atoms, a lactone, and an aromatic ring of 6 to 12 carbon atoms is further preferable. The cycloalkane of 3 to 12 carbon atoms may be monocyclic or polycyclic and is preferably polycyclic. Specific examples of the cycloalkane of 3 to 12 carbon atoms include, but not limited to, monocycloalkanes, bicycloalkanes, tricycloalkanes, and tetracycloalkanes. More specific examples thereof include, but not limited to: monocycloalkanes such as cyclopropane, cyclobutane, cyclopentane, and cyclohexane; and polycycloalkanes such as adamantane, norbornane, isobornane, tricyclodecane, and tetracyclodecane. Among them, adamantane, tricyclodecane, and tetracyclodecane are preferable, and adamantane and tricyclodecane are more preferable. The cycloalkane of 3 to 12 carbon atoms may have a substituent group. Examples of the lactone include, but not limited to, cycloalkane groups of 3 to 12 carbon atoms having a butyrolactone or lactone group. Examples of the aromatic ring of 6 to 12 carbon atoms include, but not limited to, a benzene ring, a naphthalene ring, an anthracene ring, a phenanthrene ring, and a pyrene ring. A benzene ring and a naphthalene ring are preferable, and a naphthalene ring is more preferable.

Particularly, a group of acid dissociation reactive groups selected from the group consisting of groups represented by the following formula (13-4) is preferable because of high resolution:

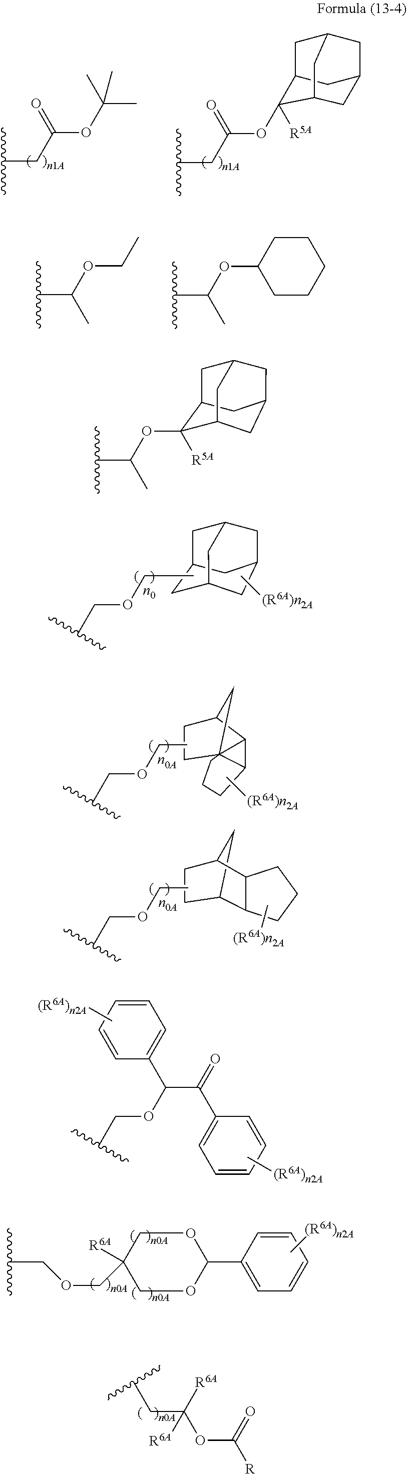

In the above formula (13-4), R^{5A} is a hydrogen atom or a linear or branched alkyl group of 1 to 4 carbon atoms; R^{6A} is a hydrogen atom, a linear or branched alkyl group of 1 to 4 carbon atoms, a cyano group, a nitro group, a heterocyclic group, a halogen atom, or a carboxyl group; n_{1A} is an integer of 0 to 4; n_{2A} is an integer of 1 to 5; and n_{0A} is an integer of 0 to 4.

Owing to the structural features mentioned above, the compound represented by the above formula (1A) has high heat resistance attributed to its rigidity despite a low molecular weight and can be used even under high temperature baking conditions. Also, the optical component forming composition of the present embodiment has such a low molecular weight and can be baked at a high temperature, while the optical component forming composition of the present embodiment has high sensitivity and can further impart a good shape to a resist pattern, because of comprising the tellurium-containing compound.

In the present embodiment, the compound represented by the above formula (1A) is preferably a compound represented by the following formula (1B) from the viewpoint of solubility in a safe solvent:

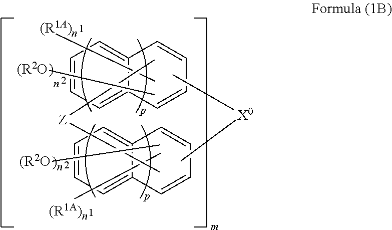

(In the formula (1B), X⁰, Z, m, and p are as defined in the above formula (A-3); each R^{1A} is independently an alkyl group, an aryl group, an alkenyl group, or a halogen atom; each R² is independently a hydrogen atom, an acid crosslinking reactive group, or an acid dissociation reactive group; each n¹ is independently an integer of 0 to (5+2×p); and each n² is independently an integer of 0 to (5+2×p), provided that at least one n² is an integer of 1 to (5+2×p).)

In the present embodiment, the compound represented by the above formula (1B) is preferably a compound represented by the following formula (2A) from the viewpoint of solubility in a safe solvent and the properties of a resist pattern:

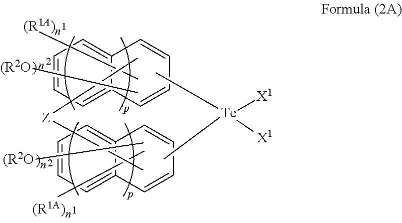

(In the formula (2A), Z, R¹, R², p, n¹, and n² are as defined in the above formula (1B); and each X¹ is independently a monovalent group containing an oxygen atom, a monovalent group containing a sulfur atom, a monovalent group containing a nitrogen atom, a hydrocarbon group, a hydrogen atom, or a halogen atom.)

In the present embodiment, the compound represented by the above formula (2A) is preferably a compound represented by the formula (2A′) given below from the viewpoint of easy physical property control. The compound represented by the above formula (2A′) is an asymmetric compound, and members in at least one of the combinations R^{1B} and R^{1B′}, n¹ and n¹′, p and p′, and the substitution position of R^{1B} and the substitution position of R^{1B′} differ from each other.

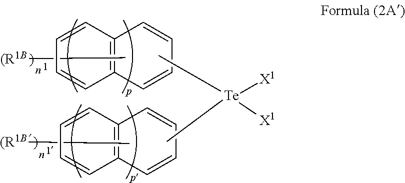

(In the formula (2A′), R^{1B} and R^{1B′} are each independently an alkyl group, an aryl group, an alkenyl group, a halogen atom, a hydroxy group or a group in which a hydrogen atom of a hydroxy group is substituted with an acid crosslinking reactive group or an acid dissociation reactive group; X¹ is as defined as X¹ in the above formula (2A); n and n^{1′} are as defined as n¹ in the above formula (2A); p and p′ are as defined as p in the above formula (2A) (i.e., each X¹ is independently a monovalent group containing an oxygen atom, a monovalent group containing a sulfur atom, a monovalent group containing a nitrogen atom, a hydrocarbon group, a hydrogen atom, or a halogen atom); and members in at least one combination selected from R^{1B} and R^{1B′}, n¹ and n^{1′}, p and p′, and the substitution position of R^{1B} and the substitution position of R^{1B′} differ from each other.)

In the present embodiment, the compound represented by the above formula (2A) is preferably a compound represented by the following formula (3A) from the viewpoint of heat resistance:

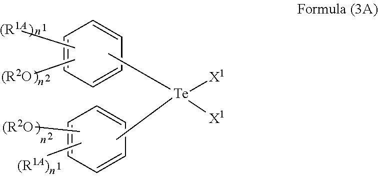

(In the formula (3A), R^{1A}, R², X¹, n¹, and n² are as defined in the above formula (2A).)

In the present embodiment, the compound represented by the above formula (3A) is preferably a compound represented by the following general formula (4A) from the viewpoint of easy production:

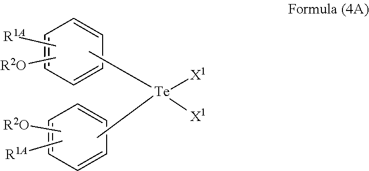

(In the formula (4A), R¹, R², and X′ are as defined above.)

In the present embodiment, X¹ in the formulae (2A), (2A′), (3A), and (4A) is more preferably a halogen atom from the viewpoint of easy production.

In the present embodiment, the compound represented by the above formula (1B) is preferably a compound represented by the following formula (2B) from the viewpoint of solubility in a safe solvent and the properties of a resist pattern:

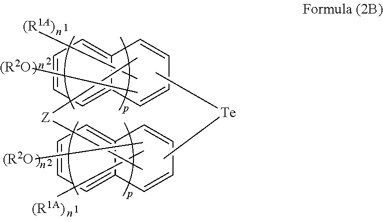

(In the formula (2B), Z, R^{1A}, R², p, n¹, and n² are as defined in the above formula (1B).)

In the present embodiment, the compound represented by the above formula (2B) is preferably a compound represented by the formula (2B′) given below from the viewpoint of easy physical property control. The compound represented by the above formula (2B′) is an asymmetric compound, and members in at least one of the combinations R^{1B} and R^{1B′}, n¹ and n¹′, p and p′, and the substitution position of R^{1B} and the substitution position of R^{1B′} differ from each other.

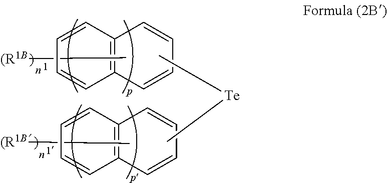

(In the formula (2B′), R^{1B} and R^{1B′} are each independently an alkyl group, an aryl group, an alkenyl group, a halogen atom, a hydroxy group or a group in which a hydrogen atom of a hydroxy group is substituted with an acid crosslinking reactive group or an acid dissociation reactive group; n¹ and n¹′ are as defined as n¹ in the above formula (2B); p and p′ are as defined as p in the above formula (2B) (i.e., p and p′ are each independently an integer of 0 to 2, and n¹ and n¹′ are each independently an integer of 0 to (5+2×p) or 0 to (5+2×p′)); and members in at least one combination selected from R^{1B} and R^{1B′}, n¹ and n¹′, p and p′, and the substitution position of R^{1B} and the substitution position of R^{1B′} differ from each other.)

In the present embodiment, the compound represented by the above formula (2B) is preferably a compound represented by the following formula (3B) from the viewpoint of heat resistance:

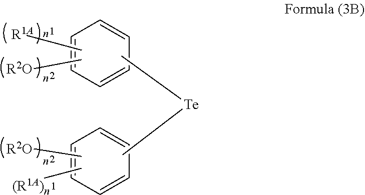

(In the formula (3B), R^{1A}, R², n¹, and n² are as defined in the above formula (2B).)

In the present embodiment, the compound represented by the above formula (3B) is preferably a compound represented by the following general formula (4B) from the viewpoint of easy production:

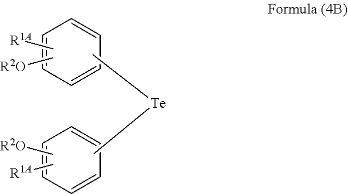

(In the formula (4B), R¹, R², and X¹ are as defined in the above formula (3B).)

In the present embodiment, in the case of forming a positive type pattern by alkaline development or in the case of forming a negative type pattern by organic development, the compound represented by the above formula (1A) preferably has at least one acid dissociation reactive group as R^{2′}. Such a tellurium-containing compound having at least one acid dissociation reactive group can be a tellurium-containing compound represented by the following formula (1A′):

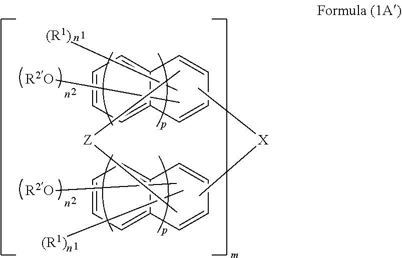

(In the formula (1A′), X, Z, m, p, R¹, n¹, and n² are as defined in the above formula (1A); and each R^{2′} is independently a hydrogen atom, an acid crosslinking reactive group, or an acid dissociation reactive group, and at least one R^{2′} is an acid dissociation reactive group.)

In the present embodiment, in the case of forming a negative type pattern by alkaline development, a tellurium-containing compound wherein all of R² are hydrogen atoms can be used as the compound represented by the above formula (1A). Such a compound can be a compound represented by the following general formula (1A″).

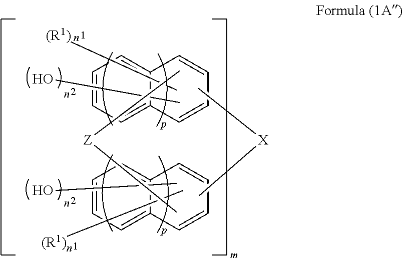

(In the above formula (1A″), X, Z, R¹, m, p, n¹, and n² are as defined in the formula (1A).)

In the present embodiment, in the case of forming a positive type pattern by alkaline development or in the case of forming a negative type pattern by organic development, the compound represented by the above formula (1B) preferably has at least one acid dissociation reactive group as R^{2′}. Such a tellurium-containing compound having at least one acid dissociation reactive group can be a tellurium-containing compound represented by the following formula (1B′):

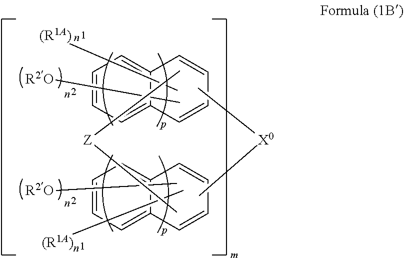

(In the formula (1B′), X⁰, Z, m, p, R^{1A}, n¹, and n² are as defined in the above formula (1B); and each R^{2′} is independently a hydrogen atom or an acid dissociation reactive group, and at least one R^{2′} is an acid dissociation reactive group.)

In the present embodiment, in the case of forming a negative type pattern by alkaline development, a tellurium-containing compound wherein all of R² are hydrogen atoms can be used as the compound represented by the above formula (1B). Such a compound can be a compound represented by the following general formula (1B″):

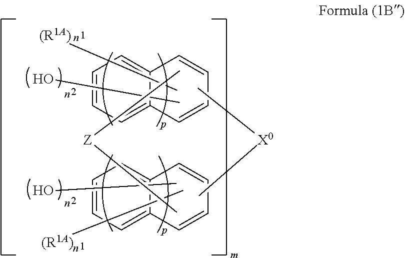

(In the formula (1B″), X⁰, Z, m, p, R^{1A}, n¹, and n² are as defined in the above formula (1B)).

In the present embodiment, a method for producing the compound represented by the above formula (A-1) is not particularly limited, and the compound represented by the above formula (A-1) can be obtained, for example, by reacting an alkoxybenzene with a corresponding tellurium halide to obtain a polyalkoxybenzene compound, subsequently performing reduction reaction with a reducing agent such as boron tribromide to obtain a polyphenol compound, and introducing an acid dissociation reactive group to at least one phenolic hydroxy group of the obtained polyphenol compound by a publicly known method.

Also, the compound represented by the above formula (A-1) can be obtained by reacting a phenol or a thiophenol with a corresponding tellurium halide to obtain a polyphenol compound, and introducing an acid dissociation reactive group to at least one phenolic hydroxy group of the obtained polyphenol compound by a publicly known method.

Further, the compound represented by the above formula (A-1) can be obtained by reacting a phenol or a thiophenol with a corresponding aldehyde containing tellurium or ketone containing tellurium in the presence of an acid or basic catalyst to obtain a polyphenol compound, and introducing an acid dissociation reactive group to at least one phenolic hydroxy group of the obtained polyphenol compound by a publicly known method.

The tellurium-containing compound can be synthesized, for example, by reacting a tellurium halide such as tellurium tetrachloride (tellurium(IV) tetrachloride) with a substituted or unsubstituted phenol derivative in the presence of a basic catalyst, as mentioned later, though the synthesis method is not particularly limited thereto. Specifically, the optical component forming composition of the present embodiment can be produced by a method for producing the optical component forming composition, comprising the step of reacting a tellurium halide with a substituted or unsubstituted phenol derivative in the presence of a basic catalyst to synthesize the tellurium-containing compound.

Upon synthesizing the compound represented by the formula (A-1), etc. by reacting a tellurium halide with a phenol, for example, a method of reacting the tellurium halide with the phenol and additionally reacting the phenol after the reaction terminates may be used. According to this method, a highly pure polyphenol compound can be obtained because a polyalkoxybenzene compound is bypassed.

In this method, for example, the tellurium halide and the phenol are reacted in an amount of 0.4 to 1.2 mol of the phenol based on 1 mol of the tellurium halide, and the phenol can be additionally reacted after the reaction terminates, from the viewpoint of obtaining the target polyphenol compound at a high yield.

Such a method can also be a method of reacting the tellurium halide with a phenol [I] and additionally reacting a phenol [II] after the reaction terminates, the phenol [I] and the phenol [II] used being different phenols, from the viewpoint of increasing the type of the polyphenol compound that can be obtained by reacting different phenols.

For such a method, it is desirable to separate a reaction intermediate after the reaction of the tellurium halide with the phenol terminates, and use only the reaction intermediate in the reaction with the phenol, from the viewpoint of obtaining a polyphenol compound with high purity. The reaction intermediate can be separated by a publicly known method. The method for separating the reaction intermediate is not particularly limited, and the intermediate can be separated by, for example, filtration.

In reaction to obtain a tellurium-containing resin from a tellurium halide and a phenol, 3 mol or more of the phenol based on 1 mol of the tellurium halide may be used from the viewpoint of improvement in yield. In the reaction to obtain a tellurium-containing resin from a tellurium halide and a phenol, a production method using 3 mol or more of the phenol based on 1 mol of the tellurium halide is particularly preferable as a method for producing the compounds represented by the formula (C1) and the formula (C2), though the production method is not limited thereto.

Examples of the tellurium halide include, but not particularly limited to, tellurium(IV) tetrafluoride, tellurium(IV) tetrachloride, tellurium(IV) tetrabromide, and tellurium(IV) tetraiodide.

Examples of the alkoxybenzene include, but not particularly limited to, methoxybenzene, dimethoxybenzene, methylmethoxybenzene, methyldimethoxybenzene, phenylmethoxybenzene, phenyldimethoxybenzene, methoxynaphthalene, dimethoxynaphthalene, ethoxybenzene, diethoxybenzene, methylethoxybenzene, methyldiethoxybenzene, phenylethoxybenzene, phenyldiethoxybenzene, ethoxynaphthalene, and diethoxynaphthalene.

Upon producing the polyalkoxybenzene compound, a reaction solvent may be used. The reaction solvent is not particularly limited as long as the reaction of the alkoxybenzene used with the corresponding tellurium halide proceeds. For example, water, methylene chloride, methanol, ethanol, propanol, butanol, tetrahydrofuran, dioxane, dimethylacetamide, N-methylpyrrolidone, or a mixed solvent thereof can be used.

The amount of the solvent is not particularly limited and can be in the range of, for example, 0 to 2000 parts by mass based on 100 parts by mass of the reaction raw materials.

Upon producing the polyphenol compound containing tellurium, the reaction temperature is not particularly limited and can be arbitrarily selected according to the reactivity of the reaction raw materials, but is preferably in the range of 10 to 200° C.

Examples of a method for producing the polyalkoxybenzene include, but not particularly limited to, a method of charging the alkoxybenzene and the corresponding tellurium halide in one portion, and a method of dropping the alkoxybenzene and the corresponding tellurium halide. After the reaction terminates, the temperature of the reaction vessel can be elevated to 130 to 230° C. in order to remove unreacted raw materials, etc. present in the system, and volatile portions can be removed at about 1 to 50 mmHg.

The amounts of the raw materials upon producing the polyalkoxybenzene compound are not particularly limited. The reaction can be proceeded by using, for example, 1 mol to an excess of the alkoxybenzene based on 1 mol of the tellurium halide, and reacting them at 20 to 150° C. at normal pressure for about 20 minutes to 100 hours.

Upon producing the polyalkoxybenzene compound, the target component can be isolated by a publicly known method after the reaction terminates. Examples of the method for isolating the target component include, but not particularly limited to, a method which involves concentrating the reaction solution, precipitating the reaction product by the addition of pure water, cooling the reaction solution to room temperature, then separating the precipitates by filtration, filtering and drying the obtained solid matter, then separating and purifying the solid matter from by-products by column chromatography, and distilling off the solvent, followed by filtration and drying to obtain the target compound.

The polyphenol compound can be obtained by reducing the polyalkoxybenzene compound. The reduction reaction can be performed using a reducing agent such as boron tribromide. Upon producing the polyphenol compound, a reaction solvent may be used. The reaction time, the reaction temperature, the amounts of raw materials, and an isolation method are not particularly limited as long as the polyphenol compound is obtained.

Examples of the phenol include, but not particularly limited to, phenol, dihydroxybenzenes, trihydroxybenzenes, naphthols, dihydroxynaphthalenes, trihydroxyanthracenes, hydroxybiphenols, dihydroxybiphenols, phenols having an alkyl group having 1 to 4 carbon atoms and/or a phenyl group at their side chains, and naphthols having an alkyl group having 1 to 4 carbon atoms and/or a phenyl group at their side chains.

A publicly known method can be used as a method for introducing an acid dissociation reactive group to at least one phenolic hydroxy group of the polyphenol compound. An acid dissociation reactive group can be introduced to at least one phenolic hydroxy group of the polyphenol compound, for example, as described below. A compound for introducing the acid dissociation reactive group can be synthesized by a publicly known method or easily obtained. Examples thereof include, but not particularly limited to, acid chlorides, acid anhydrides, active carboxylic acid derivative compounds such as dicarbonate, alkyl halides, vinyl alkyl ethers, dihydropyran, and halocarboxylic acid alkyl esters.

For example, the polyphenol compound is dissolved or suspended in an aprotic solvent such as acetone, tetrahydrofuran (THF), propylene glycol monomethyl ether acetate, dimethylacetamide, or N-methylpyrrolidone. Subsequently, a vinyl alkyl ether such as ethyl vinyl ether, or dihydropyran is added to the solution or the suspension, and the mixture is reacted at 20 to 60° C. at normal pressure for 6 to 72 hours in the presence of an acid catalyst such as pyridinium p-toluenesulfonate. The reaction solution is neutralized with an alkali compound and added to distilled water to precipitate a white solid. Then, the separated white solid can be washed with distilled water and dried to obtain the compound represented by the above formula (A-1).

The acid catalyst is not particularly limited. Inorganic acids and organic acids are widely known as well-known acid catalysts, and examples include, but not particularly limited to, inorganic acids such as hydrochloric acid, sulfuric acid, phosphoric acid, hydrobromic acid, and hydrofluoric acid; organic acids such as oxalic acid, malonic acid, succinic acid, adipic acid, sebacic acid, citric acid, fumaric acid, maleic acid, formic acid, p-toluenesulfonic acid, methanesulfonic acid, trifluoroacetic acid, dichloroacetic acid, trichloroacetic acid, trifluoromethanesulfonic acid, benzenesulfonic acid, naphthalenesulfonic acid, and naphthalenedisulfonic acid; Lewis acids such as zinc chloride, aluminum chloride, iron chloride, and boron trifluoride; and solid acids such as tungstosilicic acid, tungstophosphoric acid, silicomolybdic acid, and phosphomolybdic acid. Among them, organic acids and solid acids are preferable from the viewpoint of production, and hydrochloric acid or sulfuric acid is preferably used from the viewpoint of production such as easy availability and handleability. The acid catalysts can be used alone as one kind, or can be used in combination of two or more kinds.

Also, for example, the polyphenol compound is dissolved or suspended in an aprotic solvent such as acetone, THF, propylene glycol monomethyl ether acetate, dimethylacetamide, or N-methylpyrrolidone. Subsequently, an alkyl halide such as ethyl chloromethyl ether or a halocarboxylic acid alkyl ester such as methyladamantyl bromoacetate is added to the solution or the suspension, and the mixture is reacted at 20 to 110° C. at normal pressure for 6 hours to 72 hours in the presence of an alkali catalyst such as potassium carbonate. The reaction solution is neutralized with an acid such as hydrochloric acid and added to distilled water to precipitate a white solid. Then, the separated white solid can be washed with distilled water and dried to obtain the compound represented by the above formula (A-1).

The basic catalyst is not particularly limited and can be arbitrarily selected from well-known basic catalysts, and examples include: inorganic bases such as metal hydrides (alkali metal hydrides such as sodium hydride and potassium hydride, etc.), metal alcohol salts (alcohol salts of alkali metals such as sodium methoxide and potassium ethoxide), metal hydroxides (alkali metal or alkaline earth metal hydroxides such as sodium hydroxide and potassium hydroxide, etc.), metal carbonates (alkali metal or alkaline earth metal carbonates such as sodium carbonate and potassium carbonate, etc.), and alkali metal or alkaline earth metal bicarbonates such as sodium bicarbonate and potassium bicarbonate; and organic bases such as amines (for example, tertiary amines (trialkylamines such as triethylamine, aromatic tertiary amines such as N,N-dimethylaniline, and heterocyclic tertiary amines such as 1-methylimidazole), and carboxylic acid metal salts (acetic acid alkali metal or alkaline earth metal salts such as sodium acetate and calcium acetate, etc.). Sodium carbonate or potassium carbonate is preferable from the viewpoint of production such as easy availability and handleability. One kind or two or more kinds of the basic catalysts can be used.

The acid dissociation reactive group preferably has the property of causing chained cleavage reaction in the presence of an acid, for achieving pattern formation with higher sensitivity and higher resolution.

Specific examples of the tellurium-containing compound represented by the formula (A-1) can include the following:

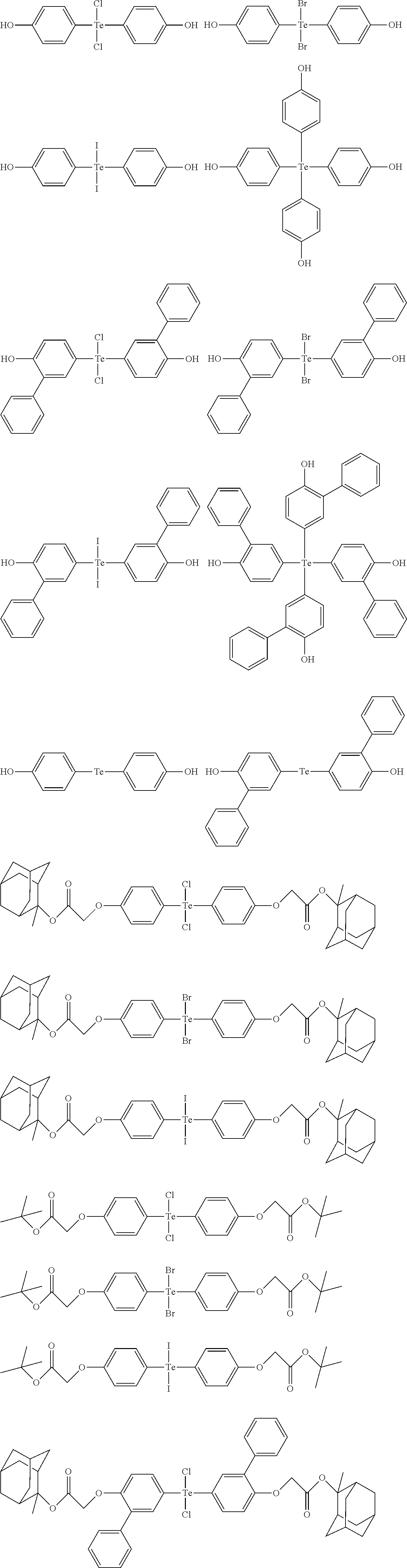

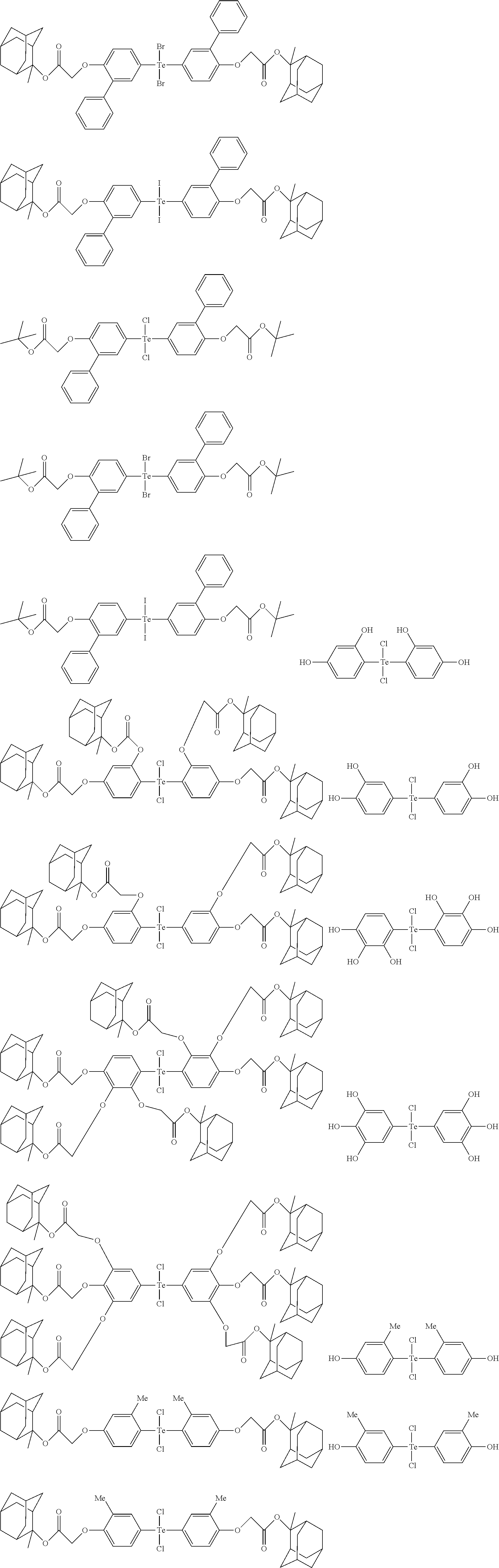

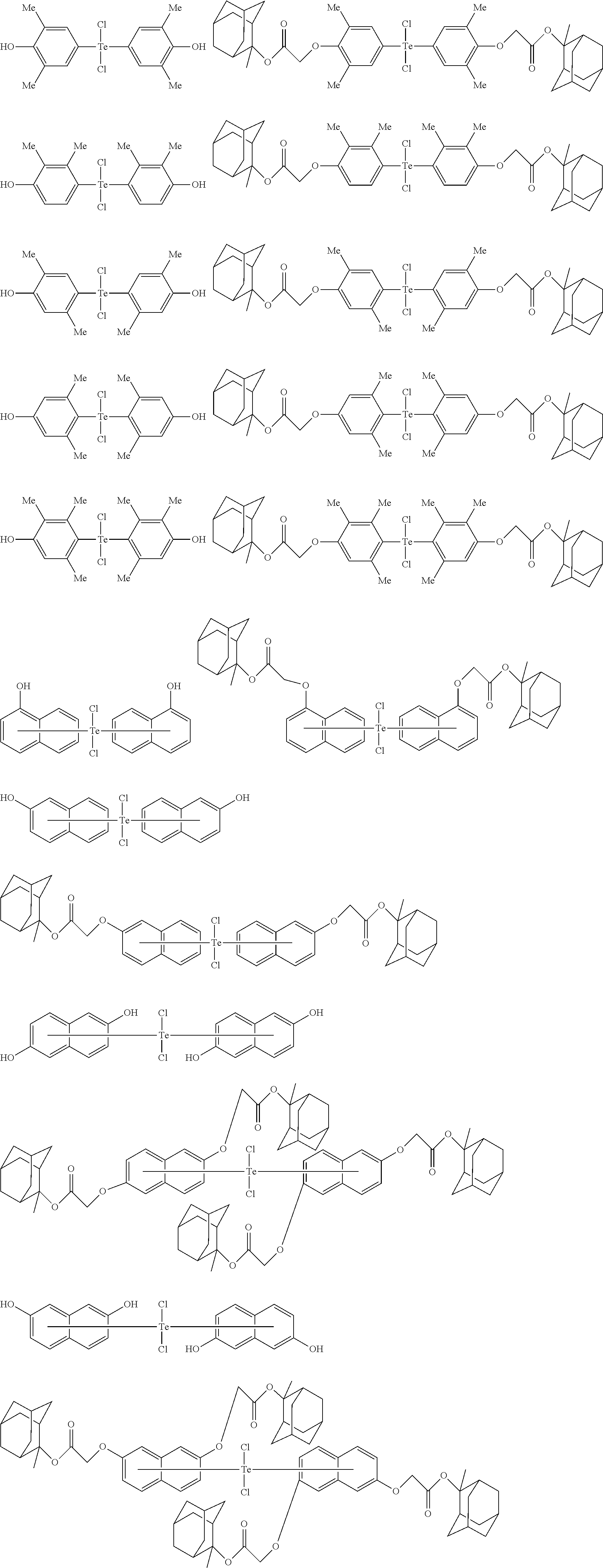

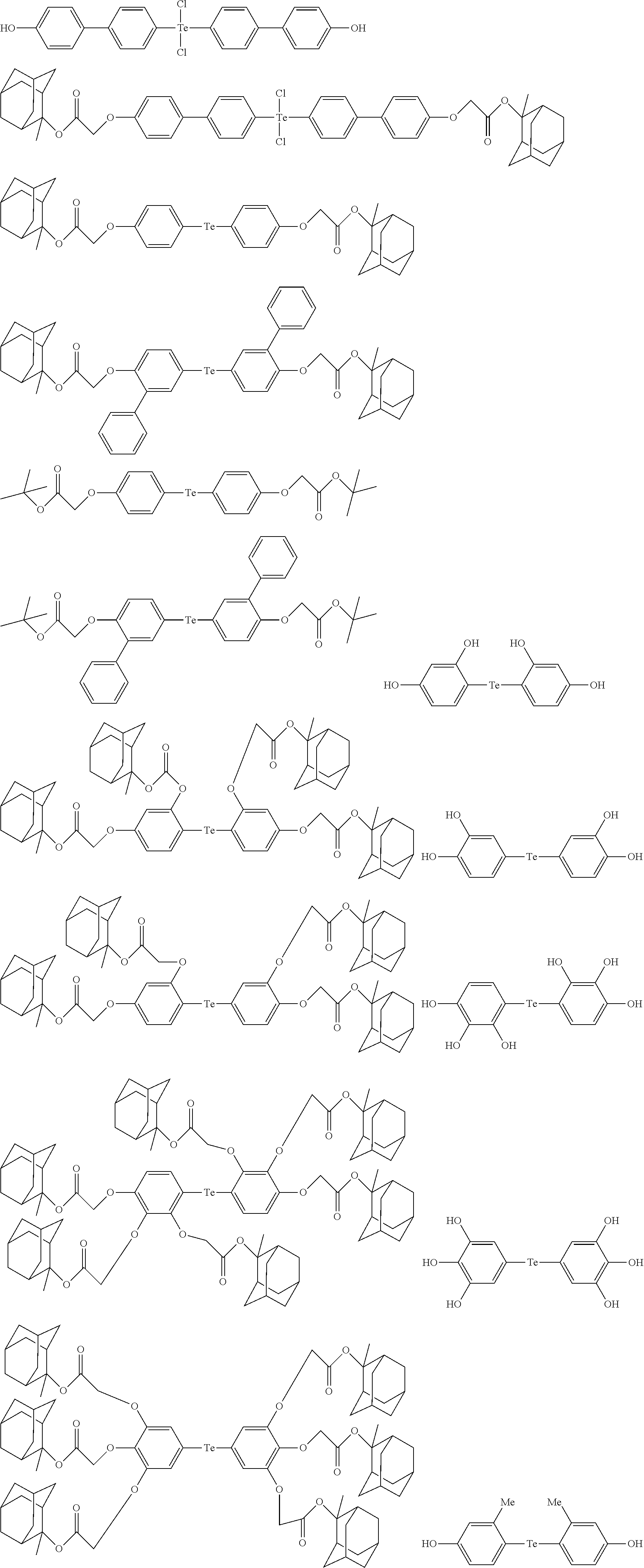

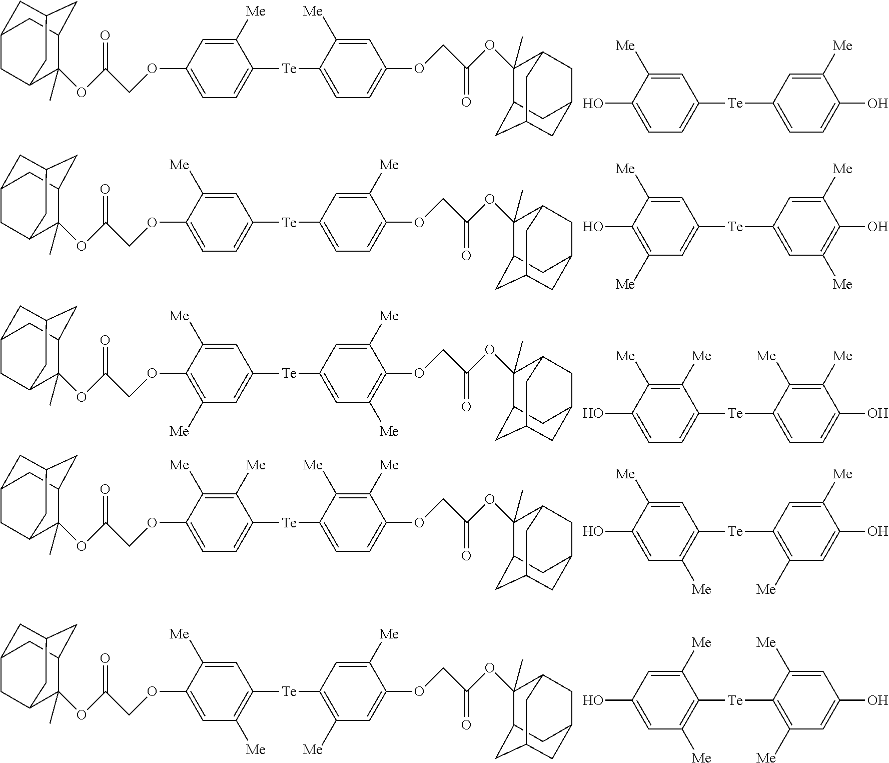

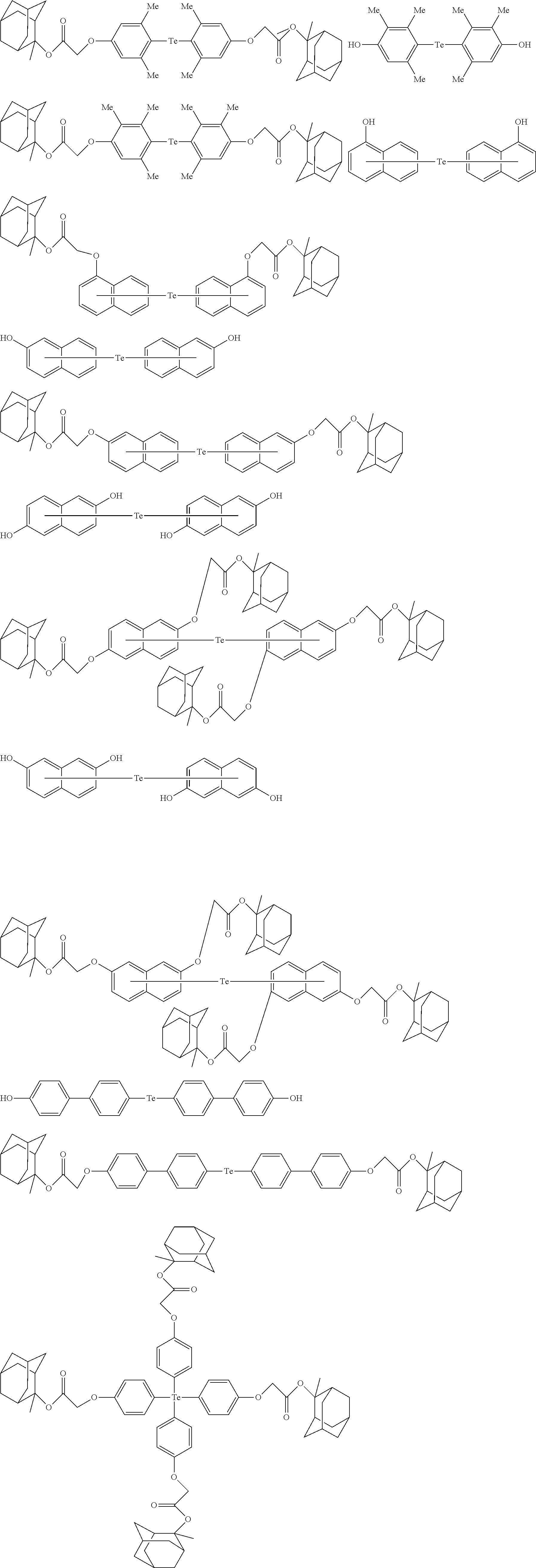

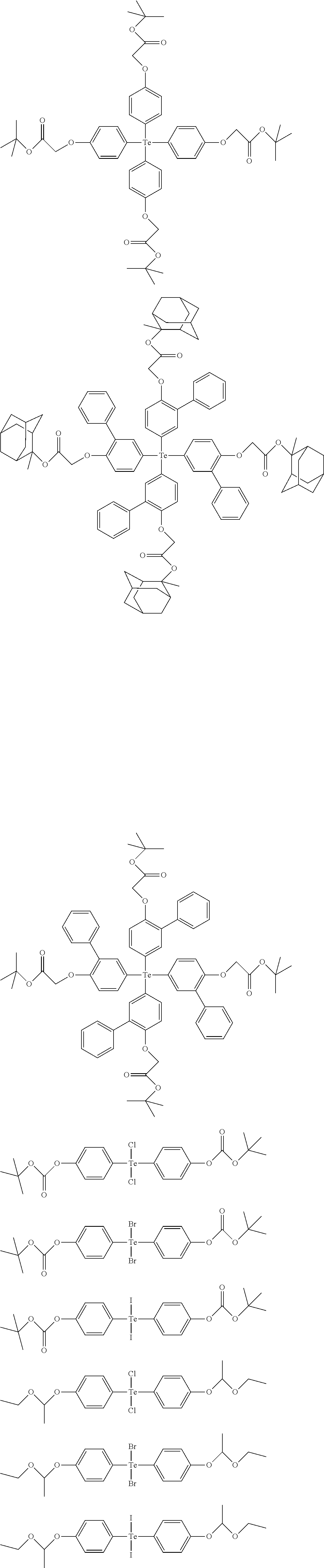

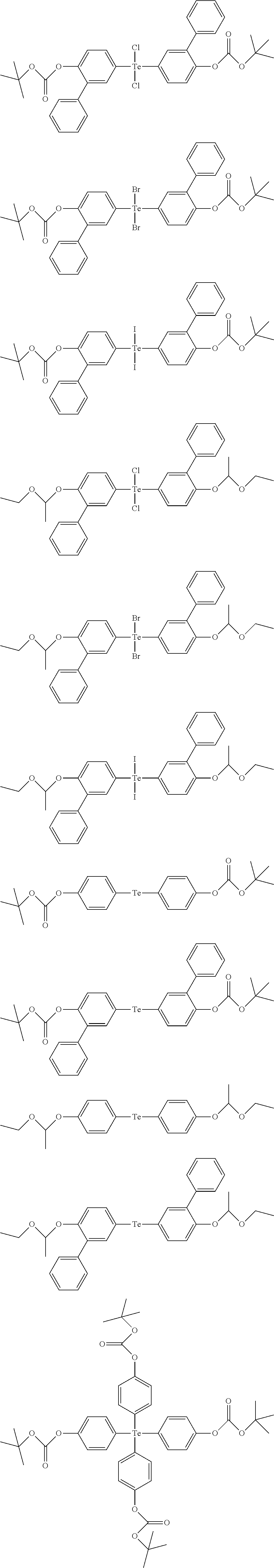

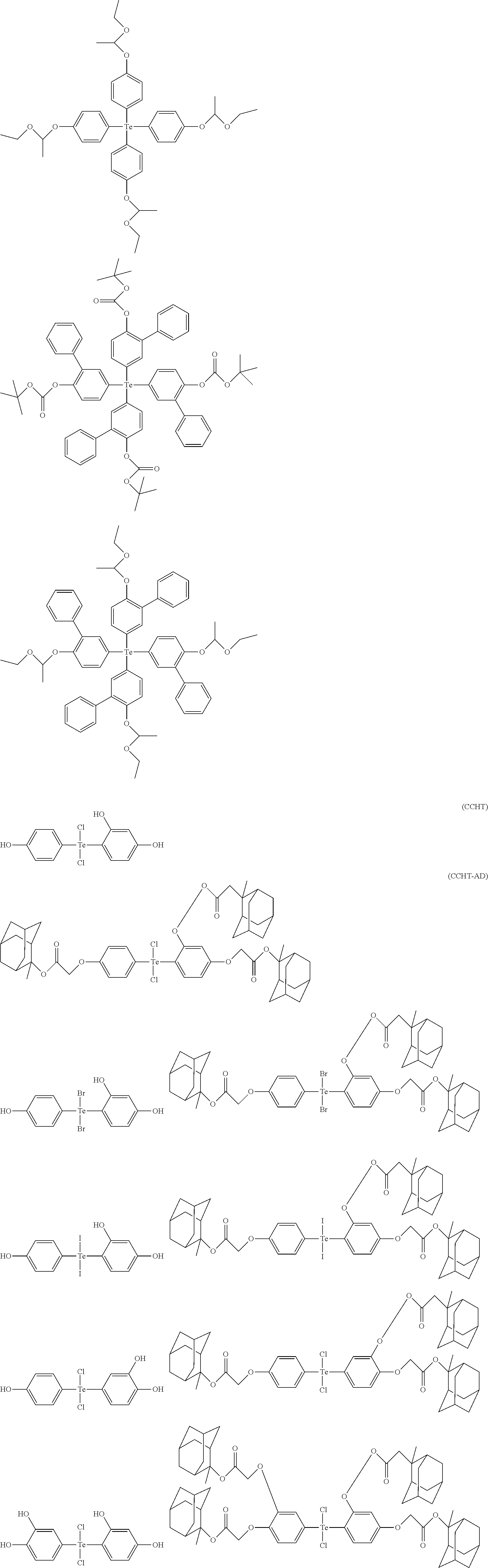

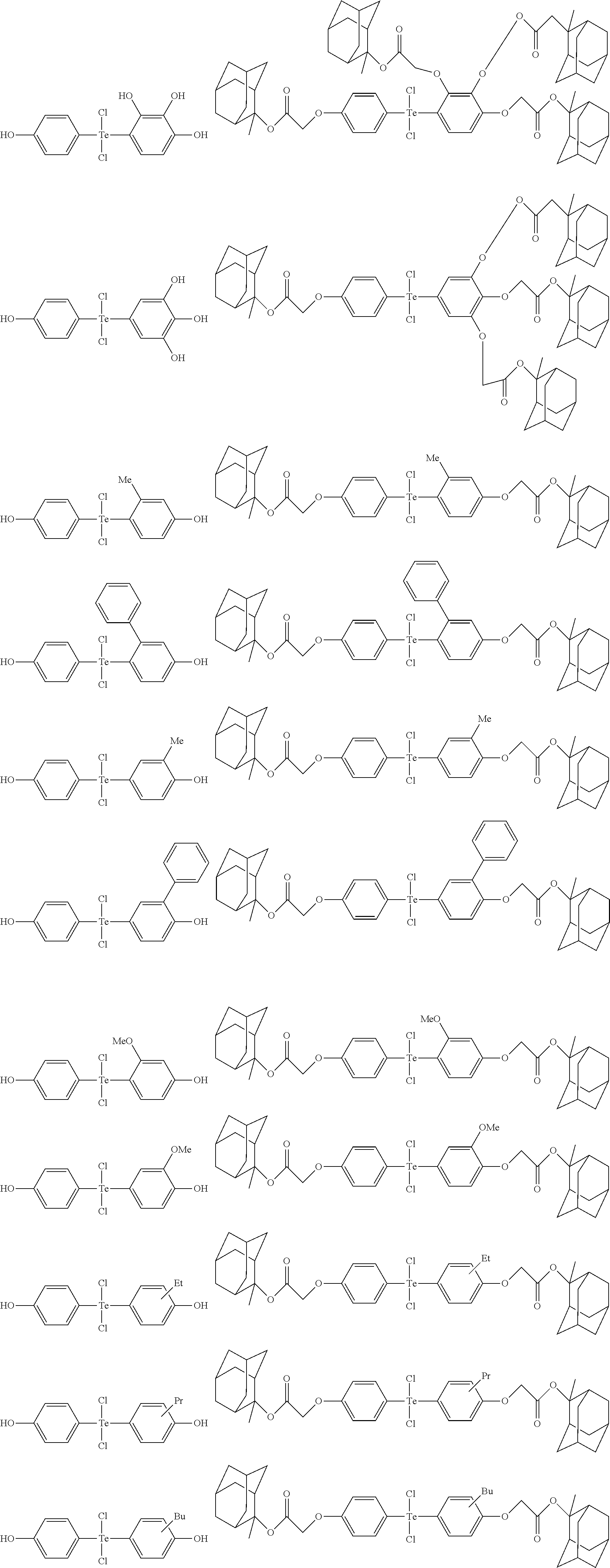

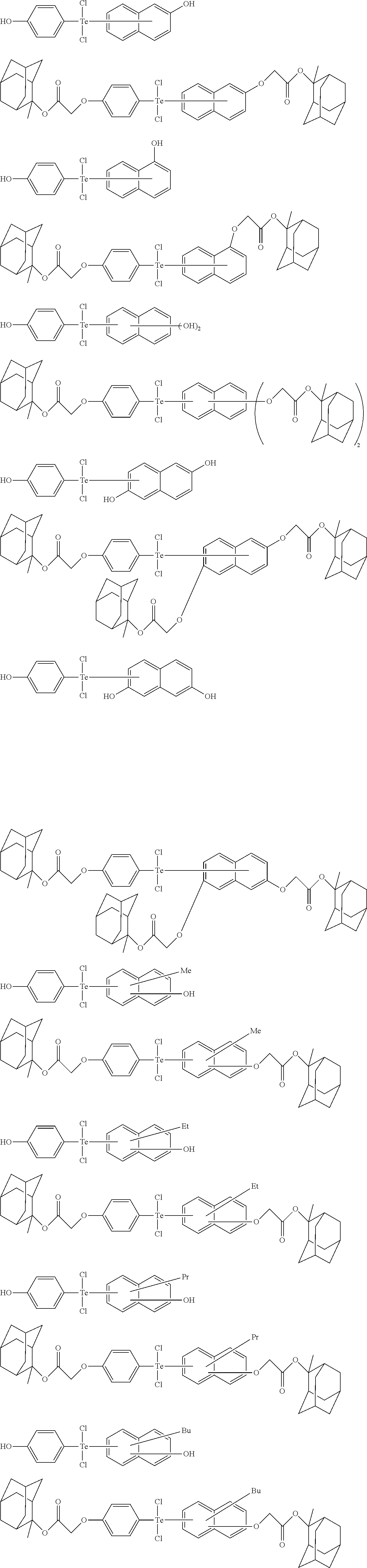

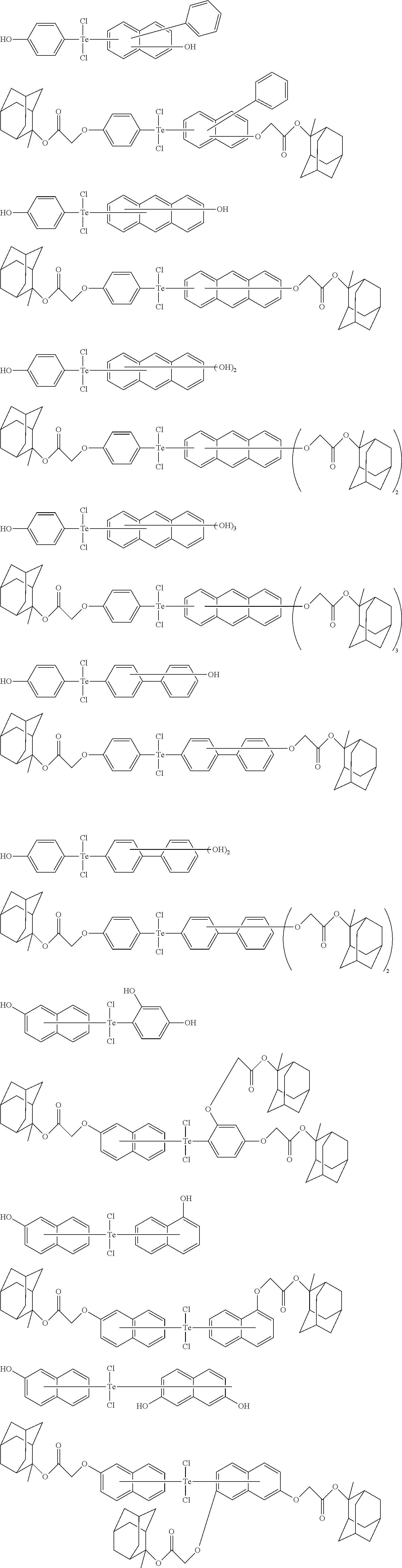

(Resin Comprising Constitutional Unit Derived from Formula (A-1))

The optical component forming composition of the present embodiment may contain a resin comprising a constitutional unit derived from the formula (A-1), instead of or together with the tellurium-containing compound represented by the formula (A-1). In other words, the optical component forming composition of the present embodiment can contain a resin obtained using the compound represented by the formula (A-1) as a monomer.

Also, the resin of the present embodiment can be obtained, for example, by reacting the compound represented by the formula (A-1) with a crosslinking compound.

As the crosslinking compound, a publicly known compound can be used without particular limitations as long as it can oligomerize or polymerize the compound represented by the formula (A-1). Specific examples thereof include, but not particularly limited to, aldehydes, ketones, carboxylic acids, carboxylic acid halides, halogen-containing compounds, amino compounds, imino compounds, isocyanates, and unsaturated hydrocarbon group-containing compounds.

As the tellurium-containing resin, for example, a resin comprising a compound derived from the compound represented by the above formula (A-1) (including, for example, a resin comprising a compound derived from the compound represented by the above formula (A-2), and a resin comprising a compound derived from the compound represented by the above formula (A-3)) as well as a resin comprising a constitutional unit represented by any of the following formulae may be used.

A resin comprising a constitutional unit represented by the following formula (B1-M):

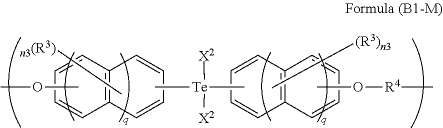

(In the formula (B1-M), each X² is independently a monovalent group containing an oxygen atom, a monovalent group containing a sulfur atom, a monovalent group containing a nitrogen atom, a hydrocarbon group, a hydrogen atom, or a halogen atom; each R³ is independently a monovalent group containing an oxygen atom, a monovalent group containing a sulfur atom, a monovalent group containing a nitrogen atom, a hydrocarbon group, or a halogen atom; q is an integer of 0 to 2; n³ is an integer of 0 to (4+2×q); and R⁴ is a single bond or any structure represented by the following general formula (5).)

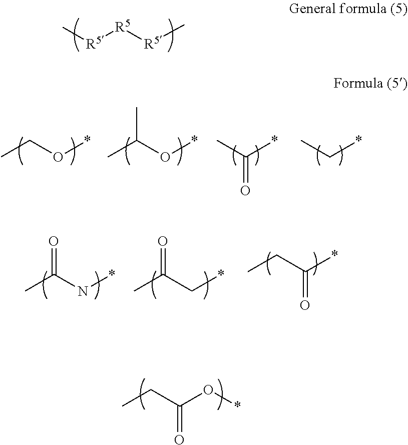

(In the general formula (5), R⁵ is a substituted or unsubstituted linear alkylene group of 1 to 20 carbon atoms, branched alkylene group of 3 to 20 carbon atoms, or cyclic alkylene group of 3 to 20 carbon atoms, or a substituted or unsubstituted arylene group of 6 to 20 carbon atoms; and each R^{5′} is independently any structure of the above formula (5′). In the formula (5′), * indicates that this portion is connected to R⁵.)

A resin comprising a constitutional unit represented by the following formula (B1-M′) (a resin wherein the R⁴ in the formula (B1-M) is a single bond):

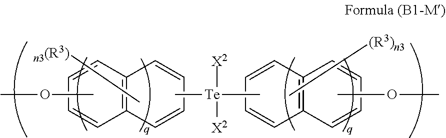

(In the formula (B1-M′), each X² is independently a monovalent group containing an oxygen atom, a monovalent group containing a sulfur atom, a monovalent group containing a nitrogen atom, a hydrocarbon group, a hydrogen atom, or a halogen atom; each R³ is independently a monovalent group containing an oxygen atom, a monovalent group containing a sulfur atom, a monovalent group containing a nitrogen atom, a hydrocarbon group, or a halogen atom; q is an integer of 0 to 2; and n³ is an integer of 0 to (4+2×q).)

A resin comprising a constitutional unit represented by the following formula (B2-M) (a resin comprising a constitutional unit wherein the R⁴ in the formula (B1-M) is any structure represented by the above general formula (5)):

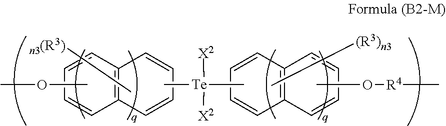

(In the formula (B2-M), X², R³, q, and n³ are as defined in the formula (B1-M); and R⁴ is any structure represented by the above general formula (5).)

A resin comprising a constitutional unit represented by the following formula (B2-M′):

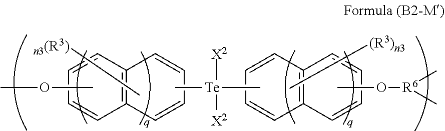

(In the formula (B2-M′), X², R³, q, and n³ are as defined in the formula (B1-M); and R⁶ is any structure represented by the following general formula (6).)

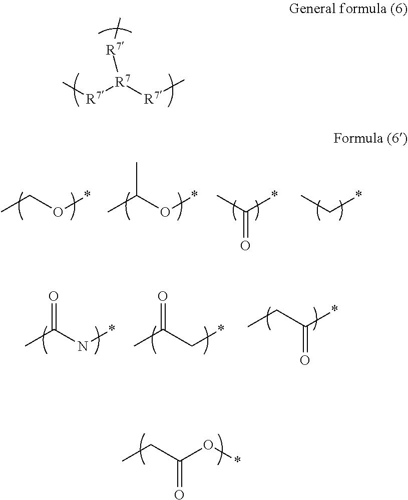

(In the general formula (6), R⁷ is a substituted or unsubstituted linear alkylene group of 1 to 20 carbon atoms, branched alkylene group of 3 to 20 carbon atoms, or cyclic alkylene group of 3 to 20 carbon atoms, or a substituted or unsubstituted arylene group of 6 to 20 carbon atoms; and each R^{7′} is independently any structure of the above formula (6′). In the formula (6′), * indicates that this portion is connected to R⁷.)

A resin comprising a constitutional unit represented by the following formula (C1):

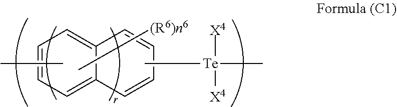

(In the formula (Cl), each X⁴ is independently a monovalent group containing an oxygen atom, a monovalent group containing a sulfur atom, a monovalent group containing a nitrogen atom, a hydrocarbon group, a hydrogen atom, or a halogen atom; each R⁶ is independently a monovalent group containing an oxygen atom, a monovalent group containing a sulfur atom, a monovalent group containing a nitrogen atom, a hydrocarbon group, or a halogen atom; r is an integer of 0 to 2; and n⁶ is an integer of 2 to (4+2×r).)

A resin comprising a constitutional unit represented by the following formula (B3-M):

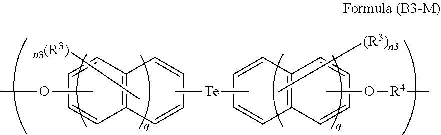

(In the formula (B3-M), each R³ is independently a monovalent group containing an oxygen atom, a monovalent group containing a sulfur atom, a monovalent group containing a nitrogen atom, a hydrocarbon group, or a halogen atom; q is an integer of 0 to 2; n³ is an integer of 0 to (4+2×q); and R⁴ is a single bond or any structure represented by the following general formula (5).)

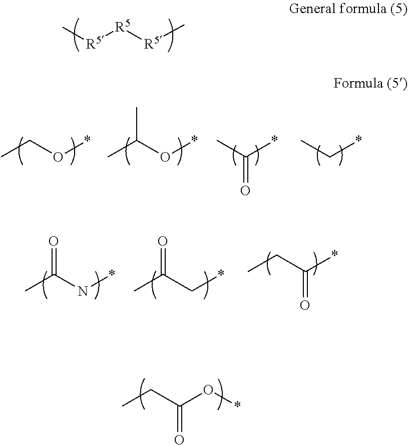

(In the general formula (5), R⁵ is a substituted or unsubstituted linear alkylene group of 1 to 20 carbon atoms, branched alkylene group of 3 to 20 carbon atoms, or cyclic alkylene group of 3 to 20 carbon atoms, or a substituted or unsubstituted arylene group of 6 to 20 carbon atoms; and each R^{5′} is independently any structure of the above formula (5′). In the formula (5′), * indicates that this portion is connected to R⁵.)

A resin comprising a constitutional unit represented by the following formula (B3-M′) (a resin wherein the R⁴ in the formula (B3-M) is a single bond):

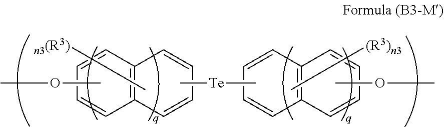

(In the formula (B3-M′), each R³ is independently a monovalent group containing an oxygen atom, a monovalent group containing a sulfur atom, a monovalent group containing a nitrogen atom, a hydrocarbon group, or a halogen atom; q is an integer of 0 to 2; and n³ is an integer of 0 to (4+2×q).)

A resin comprising a constitutional unit represented by the following formula (B4-M) (a resin comprising a constitutional unit wherein the R⁴ in the formula (B3-M) is any structure represented by the above general formula (5)):

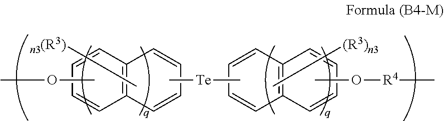

(In the formula (B4-M), R³, q, and n³ are as defined in the formula (B3-M); and R⁴ is any structure represented by the above general formula (5).)

A resin comprising a constitutional unit represented by the following formula (B4-M′):

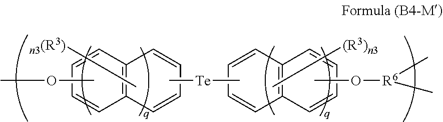

(In the formula (B4-M′), R³, q, and n³ are as defined in the formula (B3-M); and R⁶ is any structure represented by the following general formula (6).)

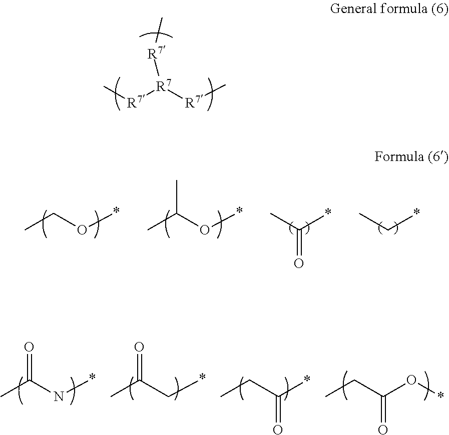

(In the general formula (6), R⁷ is a substituted or unsubstituted linear alkylene group of 1 to 20 carbon atoms, branched alkylene group of 3 to 20 carbon atoms, or cyclic alkylene group of 3 to 20 carbon atoms, or a substituted or unsubstituted arylene group of 6 to 20 carbon atoms; and each R^{7′} is independently any structure of the above formula (6′). In the formula (6′), * indicates that this portion is connected to R⁷.)

A resin comprising a constitutional unit represented by the following formula (C2):

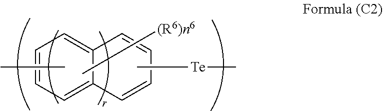

(In the formula (C2), each R⁶ is independently a monovalent group containing an oxygen atom, a monovalent group containing a sulfur atom, a monovalent group containing a nitrogen atom, a hydrocarbon group, or a halogen atom; r is an integer of 0 to 2; and n⁶ is an integer of 2 to (4+2×r).)

The resin comprising each of the constitutional units mentioned above may differ in each substituent group among the constitutional units. For example, R⁵ in the general formula (5) for R⁴ in the formula (B1-M) or (B3-M), or R⁶ in the general formula (6) for the formula (B2-M′) or (B4-M′) may be the same or different among the constitutional units.

Specific examples of the constitutional unit derived from the formula (A-1) can include the following:

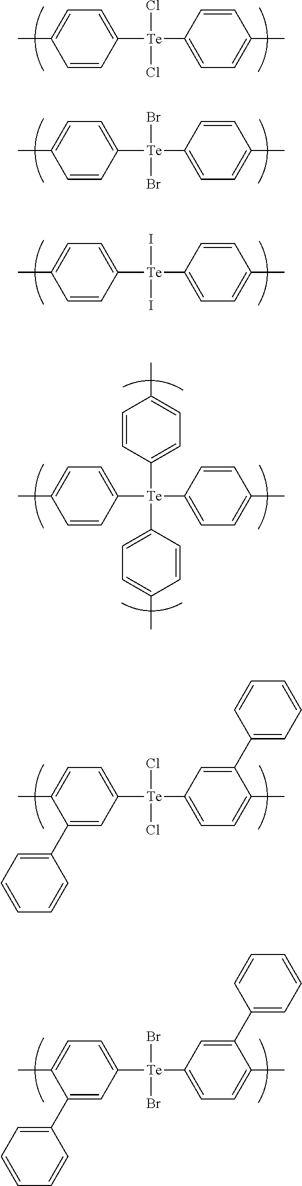

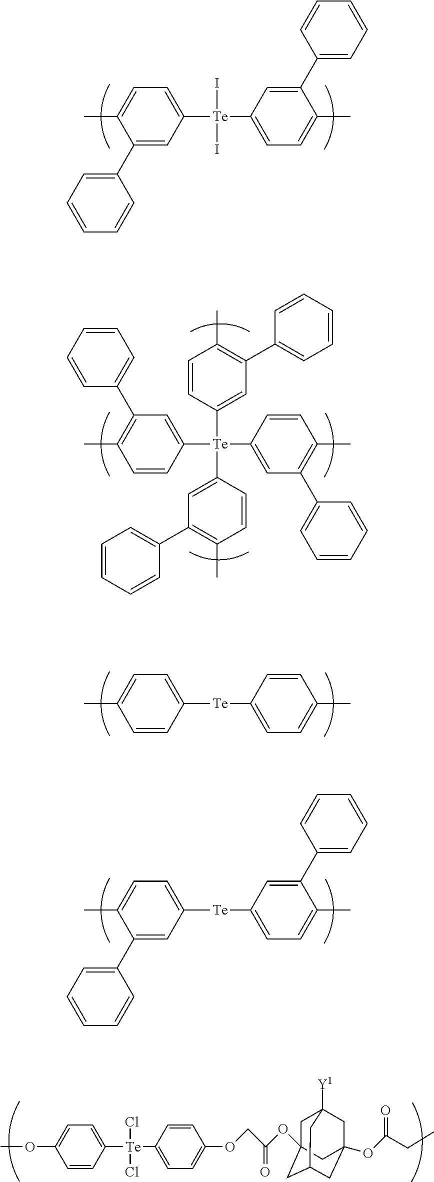

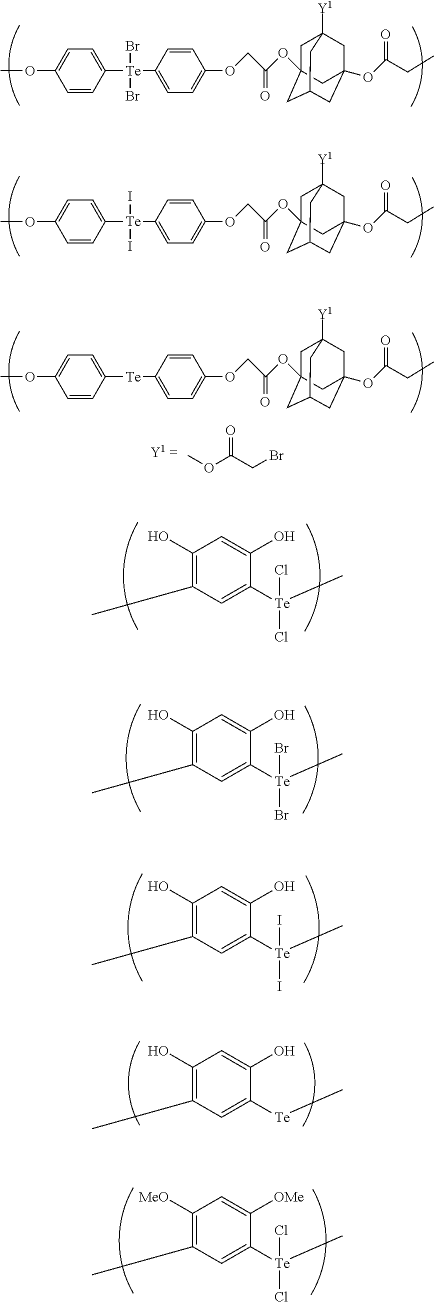

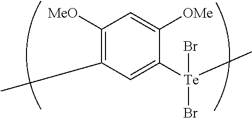

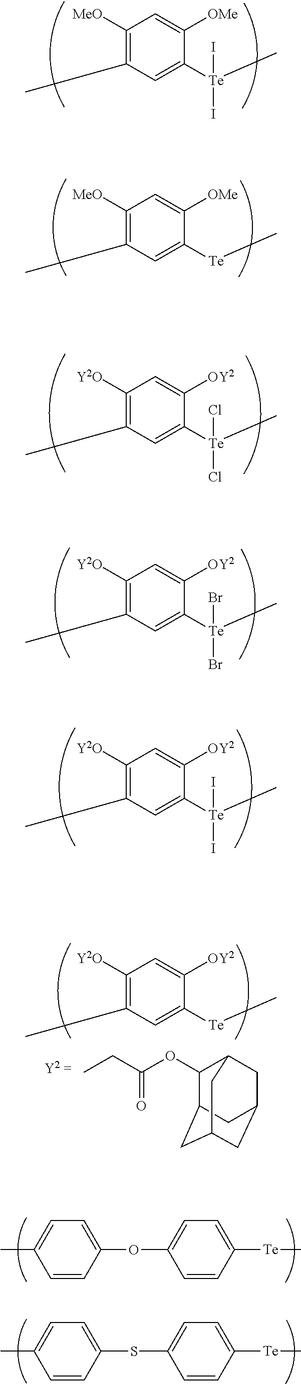

Herein, the resin according to the present embodiment may be a homopolymer of the compound represented by the above formula (A-1), but may be a copolymer with an additional phenol. Herein, examples of the copolymerizable phenol include, but not particularly limited to, phenol, cresol, dimethylphenol, trimethylphenol, butylphenol, phenylphenol, diphenylphenol, naphthylphenol, resorcinol, methylresorcinol, catechol, butylcatechol, methoxyphenol, methoxyphenol, propylphenol, pyrogallol, and thymol.

Alternatively, the resin according to the present embodiment may be a copolymer with a polymerizable monomer, instead of the additional phenol mentioned above. Examples of such a monomer for copolymerization include, but not particularly limited to, naphthol, methylnaphthol, methoxynaphthol, dihydroxynaphthalene, indene, hydroxyindene, benzofuran, hydroxyanthracene, acenaphthylene, biphenyl, bisphenol, trisphenol, dicyclopentadiene, tetrahydroindene, 4-vinylcyclohexene, norbornadiene, vinylnorbornene, pinene, and limonene. The resin according to the present embodiment may be a binary or higher (e.g., binary to quaternary) copolymer of the compound represented by the above formula (A-1) and the phenol mentioned above, may be a binary or higher (e.g., binary to quaternary) copolymer of the compound represented by the above formula (A-1) and the monomer for copolymerization mentioned above, or may be a ternary or higher (e.g., ternary to quaternary) copolymer of the compound represented by the above formula (A-1), the phenol mentioned above, and the monomer for copolymerization mentioned above.

The molecular weight of the resin according to the present embodiment is not particularly limited, and the weight average molecular weight (Mw) in terms of polystyrene is preferably 500 to 30,000 and more preferably 750 to 20,000. The resin according to the present embodiment preferably has a dispersibility (weight average molecular weight Mw/number average molecular weight Mn) within the range of 1.2 to 7 from the viewpoint of enhancing crosslinking efficiency and suppressing volatile components during baking. The Mn can be determined by a method described in Examples mentioned later.

The compound represented by the above formula (A-1) and/or the resin obtained with the compound as a constitutional unit preferably has high solubility in a solvent from the viewpoint of easier application to a wet process, etc. More specifically, in the case of using 1-methoxy-2-propanol (PGME) and/or propylene glycol monomethyl ether acetate (PGMEA) as a solvent, the solubility of the compound and/or the resin in the solvent is preferably 10% by mass or more. Herein, the solubility in PGME and/or PGMEA is defined as “Mass of the resin (or the compound)/(Mass of the resin (or the compound)+Mass of the solvent)×100 (% by mass)”. For example, 10 g of the compound represented by the above formula (A-1) and/or the resin obtained with the compound as a monomer is evaluated as being dissolved in 90 g of PGMEA when the solubility of the compound represented by the formula (A-1) and/or the resin obtained with the compound as a monomer in PGMEA is “3% by mass or more”, and is evaluated as being not dissolved therein when the solubility is “less than 3% by mass”

### [Method for Purifying Compound or Resin]

The compound or the resin of the present embodiment can be purified by a purification method comprising the following steps.

Specifically, the purification method comprises the steps of: obtaining a solution (A) by dissolving the compound represented by the formula (A-1) or the resin comprising a constitutional unit derived from the formula (A-1) in a solvent comprising an organic solvent that does not inadvertently mix with water; and extracting impurities in the compound represented by the above formula (A-1) or the resin by bringing the obtained solution (A) into contact with an acidic aqueous solution (a first extraction step).

In the case of using the purification method of the present embodiment, the resin is preferably a resin obtained by reacting the compound represented by the formula (A-1) with a crosslinking compound.

According to the purification method of the present embodiment, the contents of various metals that may be contained as impurities in the compound or the resin having a specific structure described above can be effectively reduced.

Metals contained in the solution (A) containing the compound represented by the formula (A-1) or the resin comprising a constitutional unit derived from the compound represented by the formula (A-1) are transferred to the aqueous phase, then the organic phase and the aqueous phase are separated, and thus the compound represented by the formula (A-1) or the resin comprising a constitutional unit derived from the compound represented by the formula (A-1) having a reduced metal content can be obtained.

The compound represented by the formula (A-1) or the resin comprising a constitutional unit derived from the compound represented by the formula (A-1) used in the purification method of the present embodiment may be alone, or may be a mixture of two or more kinds. Also, the compound represented by the formula (A-1) or the resin comprising a constitutional unit derived from the compound represented by the formula (A-1) may be applied to the production method of the present embodiment together with various surfactants, various crosslinking agents, various acid generators, various stabilizers, and the like.

The “organic solvent that does not inadvertently mix with water” used in the purification method of the present embodiment means an organic solvent that does not uniformly mix with water at any ratio. Such an organic solvent is not particularly limited, but is preferably an organic solvent that is safely applicable to semiconductor manufacturing processes, and specifically it is an organic solvent having a solubility in water at room temperature of less than 30%, and more preferably is an organic solvent having a solubility of less than 20% and particularly preferably less than 10%. The amount of the organic solvent used is preferably 1 to 100 parts by mass based on 100 parts by mass of the compound represented by the formula (A-1) and the resin comprising a constitutional unit derived from the compound represented by the formula (A-1) used.

Specific examples of the organic solvent that does not inadvertently mix with water include, but not limited to, ethers such as diethyl ether and diisopropyl ether; esters such as ethyl acetate, n-butyl acetate, and isoamyl acetate; ketones such as methyl ethyl ketone, methyl isobutyl ketone, ethyl isobutyl ketone, cyclohexanone (CHN), cyclopentanone, 2-heptanone, and 2-pentanone; glycol ether acetates such as ethylene glycol monoethyl ether acetate, ethylene glycol monobutyl ether acetate, propylene glycol monomethyl ether acetate (PGMEA), and propylene glycol monoethyl ether acetate; aliphatic hydrocarbons such as n-hexane and n-heptane; aromatic hydrocarbons such as toluene and xylene; and halogenated hydrocarbons such as methylene chloride and chloroform. Among these, one or more organic solvents selected from the group consisting of toluene, 2-heptanone, cyclohexanone, cyclopentanone, methyl isobutyl ketone, propylene glycol monomethyl ether acetate, ethyl acetate, and the like are preferable, methyl isobutyl ketone, ethyl acetate, cyclohexanone, and propylene glycol monomethyl ether acetate are more preferable, and methyl isobutyl ketone and ethyl acetate are still more preferable. Methyl isobutyl ketone, ethyl acetate, and the like have relatively high saturation solubility for the compound represented by the formula (A-1) or the resin comprising a constitutional unit derived from the compound represented by the formula (A-1) and a relatively low boiling point, and it is thus possible to reduce the load in the case of industrially distilling off the solvent and in the step of removing the solvent by drying.

These organic solvents can be each used alone, and can be used as a mixture of two or more kinds.

The “acidic aqueous solution” used in the purification method of the present embodiment is arbitrarily selected from aqueous solutions in which generally known organic compounds or inorganic compounds are dissolved in water. Examples of the acidic aqueous solution include, but not limited to, aqueous mineral acid solutions in which mineral acids such as hydrochloric acid, sulfuric acid, nitric acid, and phosphoric acid are dissolved in water; and aqueous organic acid solutions in which organic acids such as acetic acid, propionic acid, oxalic acid, malonic acid, succinic acid, fumaric acid, maleic acid, tartaric acid, citric acid, methanesulfonic acid, phenolsulfonic acid, p-toluenesulfonic acid, and trifluoroacetic acid are dissolved in water. These acidic aqueous solutions can be each used alone, and can be also used as a combination of two or more kinds. Among these acidic aqueous solutions, aqueous solutions of one or more mineral acids selected from the group consisting of hydrochloric acid, sulfuric acid, nitric acid, and phosphoric acid, or aqueous solutions of one or more organic acids selected from the group consisting of acetic acid, propionic acid, oxalic acid, malonic acid, succinic acid, fumaric acid, maleic acid, tartaric acid, citric acid, methanesulfonic acid, phenolsulfonic acid, p-toluenesulfonic acid, and trifluoroacetic acid are preferable, aqueous solutions of sulfuric acid, nitric acid, and carboxylic acids such as acetic acid, oxalic acid, tartaric acid, and citric acid are more preferable, aqueous solutions of sulfuric acid, oxalic acid, tartaric acid, and citric acid are still more preferable, and an aqueous solution of oxalic acid is further preferable. It is considered that polyvalent carboxylic acids such as oxalic acid, tartaric acid, and citric acid coordinate with metal ions and provide a chelating effect, and thus tend to be capable of more effectively removing metals. As for water used herein, it is preferable to use water, the metal content of which is small, such as ion exchanged water, according to the purpose of the purification method of the present embodiment.

The pH of the acidic aqueous solution used in the purification method of the present embodiment is not particularly limited, but it is preferable to regulate the acidity of the aqueous solution in consideration of an influence on the compound represented by the formula (A-1) or the resin comprising a constitutional unit derived from the compound represented by the formula (A-1). Normally, the pH range of the acidic aqueous solution is about 0 to 5, and is preferably about pH 0 to 3.

The amount of the acidic aqueous solution used in the purification method of the present embodiment is not particularly limited, but it is preferable to regulate the amount from the viewpoint of reducing the number of extraction operations for removing metals and from the viewpoint of ensuring operability in consideration of the overall amount of fluid. From the above viewpoints, the amount of the acidic aqueous solution used is preferably 10 to 200% by mass, more preferably 20 to 100% by mass, based on 100% by mass of the solution (A).

In the purification method of the present embodiment, by bringing an acidic aqueous solution as described above into contact with the solution (A) containing one or more selected from the compound represented by the formula (A-1) and the resin comprising a constitutional unit derived from the compound represented by the formula (A-1) and the organic solvent that does not inadvertently mix with water, metals can be extracted from the compound or the resin in the solution (A).

When an organic solvent that advertently mixes with water is contained, there is a tendency that the amount of the compound represented by the formula (A-1) or the resin comprising a constitutional unit derived from the compound represented by the formula (A-1) charged can be increased, also the fluid separability is improved, and purification can be carried out at a high reaction vessel efficiency. The method for adding the organic solvent that advertently mixes with water is not particularly limited. For example, any of a method involving adding it to the organic solvent-containing solution in advance, a method involving adding it to water or the acidic aqueous solution in advance, and a method involving adding it after bringing the organic solvent-containing solution into contact with water or the acidic aqueous solution. Among these, the method involving adding it to the organic solvent-containing solution in advance is preferable in terms of the workability of operations and the ease of managing the amount.

The organic solvent that inadvertently mixes with water used in the purification method of the present embodiment is not particularly limited, but is preferably an organic solvent that is safely applicable to semiconductor manufacturing processes. The amount of the organic solvent used that inadvertently mixes with water is not particularly limited as long as the solution phase and the aqueous phase separate, but is preferably 0.1 to 100 parts by mass, more preferably 0.1 to 50 parts by mass, and further preferably 0.1 to 20 parts by mass based on 100 parts by mass of the compound represented by the formula (A-1) and the resin comprising a constitutional unit derived from the compound represented by the formula (A-1).

Specific examples of the organic solvent used in the purification method of the present embodiment that inadvertently mixes with water include, but not limited to, ethers such as tetrahydrofuran and 1,3-dioxolane; alcohols such as methanol, ethanol, and isopropanol; ketones such as acetone and N-methylpyrrolidone; aliphatic hydrocarbons such as glycol ethers such as ethylene glycol monoethyl ether, ethylene glycol monobutyl ether, propylene glycol monomethyl ether (PGME), and propylene glycol monoethyl ether. Among these, N-methylpyrrolidone, propylene glycol monomethyl ether, and the like are preferable, and N-methylpyrrolidone and propylene glycol monomethyl ether are more preferable. These solvents can be each used alone, and can be used as a mixture of two or more kinds.

In the purification method of the present embodiment, the temperature when the solution (A) and the acidic aqueous solution are brought into contact, i.e., when extraction treatment is carried out, is preferably in the range of 20 to 90° C., and more preferably 30 to 80° C. The extraction operation is not particularly limited, and is carried out, for example, by thoroughly mixing the solution (A) and the acidic aqueous solution by stirring or the like and then leaving the obtained mixed solution to stand still. Thereby, metals contained in the solution (A) containing one or more selected from the compound represented by the formula (A-1) and the resin comprising a constitutional unit derived from the compound represented by the formula (A-1) and the organic solvents are transferred to the aqueous phase. Also, by this operation, the acidity of the solution (A) is lowered, and the degradation of the compound represented by the formula (A-1) and the resin comprising a constitutional unit derived from the compound represented by the formula (A-1) can be suppressed.

By being left to stand still, the mixed solution is separated into an aqueous phase and a solution phase containing one or more selected from the compound represented by the formula (A-1) and the resin comprising a constitutional unit derived from the compound represented by the formula (A-1) and the organic solvents, and thus the solution phase containing one or more selected from the compound represented by the formula (A-1) and the resin comprising a constitutional unit derived from the compound represented by the formula (A-1) and the organic solvents can be recovered by decantation. The time for leaving the mixed solution to stand still is not particularly limited, but it is preferable to regulate the time for leaving the mixed solution to stand still from the viewpoint of attaining good separation of the solution phase containing the organic solvents and the aqueous phase. Normally, the time for leaving the mixed solution to stand still is 1 minute or longer, preferably 10 minutes or longer, and more preferably 30 minutes or longer. While the extraction treatment may be carried out once, it is effective to repeat mixing, leaving-to-stand-still, and separating operations multiple times.

It is preferable that the purification method of the present embodiment includes the step of extracting impurities in the compound or the resin by further bringing the solution phase containing the compound or the resin into contact with water after the first extraction step (the second extraction step).

Specifically, for example, it is preferable that after the extraction treatment is carried out using an acidic aqueous solution, the solution phase that is extracted and recovered from the aqueous solution and that contains one or more selected from the compound represented by the formula (A-1) and the resin comprising a constitutional unit derived from the compound represented by the formula (A-1) and the organic solvents is further subjected to extraction treatment with water. The extraction treatment with water is not particularly limited, and can be carried out, for example, by thoroughly mixing the solution phase and water by stirring or the like and then leaving the obtained mixed solution to stand still. The mixed solution after being left to stand still is separated into an aqueous phase and a solution phase containing one or more selected from the compound represented by the formula (A-1) and the resin comprising a constitutional unit derived from the compound represented by the formula (A-1) and the organic solvents, and thus the solution phase containing one or more selected from the compound represented by the formula (A-1) and the resin comprising a constitutional unit derived from the compound represented by the formula (A-1) and the organic solvents can be recovered by decantation.

Water used herein is preferably water, the metal content of which is small, such as ion exchanged water, according to the purpose of the present embodiment. While the extraction treatment may be carried out once, it is effective to repeat mixing, leaving-to-stand-still, and separating operations multiple times. The proportions of both used in the extraction treatment and temperature, time, and other conditions are not particularly limited, and may be the same as those of the previous contact treatment with the acidic aqueous solution.

Water that is possibly present in the thus-obtained solution containing one or more selected from the compound represented by the formula (A-1) and the resin comprising a constitutional unit derived from the compound represented by the formula (A-1) and the organic solvents can be easily removed by performing vacuum distillation or a like operation. Also, if required, the concentration of the compound represented by the formula (A-1) and the resin comprising a constitutional unit derived from the compound represented by the formula (A-1) can be regulated to be any concentration by adding an organic solvent to the solution.

The method for isolating one or more selected from the compound represented by the above formula (A-1) and the resin comprising a constitutional unit derived from the compound represented by the formula (A-1) from the obtained solution containing one or more selected from the compound represented by the formula (A-1) and the resin comprising a constitutional unit derived from the compound represented by the formula (A-1) and the organic solvents is not particularly limited, and publicly known methods can be carried out, such as reduced-pressure removal, separation by reprecipitation, and a combination thereof. Publicly known treatments such as concentration operation, filtration operation, centrifugation operation, and drying operation can be carried out if required.

### (Physical Properties and the Like of Optical Component Forming Composition)

The optical component forming composition of the present embodiment can form an amorphous film by a publicly known method such as spin coating.

### (Other Components of Optical Component Forming Composition)

The optical component forming composition of the present embodiment contains at least any one of the tellurium-containing compound and the tellurium-containing resin, preferably the compound represented by the formula (A-1) and the resin comprising a constitutional unit derived from the compound represented by the formula (A-1), as a solid component. The optical component forming composition of the present embodiment may contain both the compound represented by the formula (A-1) and the resin comprising a constitutional unit derived from the compound represented by the formula (A-1).

### (Solvent)

It is preferable that the optical component forming composition of the present embodiment further contains a solvent other than the compound represented by the formula (A-1) and the resin comprising a constitutional unit derived from the compound represented by the formula (A-1).

Examples of the solvent used in the optical component forming composition of the present embodiment can include, but not particularly limited to, ethylene glycol monoalkyl ether acetates such as ethylene glycol monomethyl ether acetate, ethylene glycol monoethyl ether acetate, ethylene glycol mono-n-propyl ether acetate, and ethylene glycol mono-n-butyl ether acetate; ethylene glycol monoalkyl ethers such as ethylene glycol monomethyl ether and ethylene glycol monoethyl ether; propylene glycol monoalkyl ether acetates such as propylene glycol monomethyl ether acetate, propylene glycol monoethyl ether acetate (PGMEA), propylene glycol mono-n-propyl ether acetate, and propylene glycol mono-n-butyl ether acetate; propylene glycol monoalkyl ethers such as propylene glycol monomethyl ether (PGME) and propylene glycol monoethyl ether; ester lactates such as methyl lactate, ethyl lactate, n-propyl lactate, n-butyl lactate, and n-amyl lactate; aliphatic carboxylic acid esters such as methyl acetate, ethyl acetate, n-propyl acetate, n-butyl acetate, n-amyl acetate, n-hexyl acetate, methyl propionate, and ethyl propionate; other esters such as methyl 3-methoxypropionate, ethyl 3-methoxypropionate, methyl 3-ethoxypropionate, ethyl 3-ethoxypropionate, methyl 3-methoxy-2-methylpropionate, 3-methoxybutylacetate, 3-methyl-3-methoxybutylacetate, butyl 3-methoxy-3-methylpropionate, butyl 3-methoxy-3-methylbutyrate, methyl acetoacetate, methyl pyruvate, and ethyl pyruvate; aromatic hydrocarbons such as toluene and xylene; ketones such as methyl ethyl ketone, 2-heptanone, 3-heptanone, 4-heptanone, cyclopentanone (CPN), and cyclohexanone (CHN); amides such as N,N-dimethylformamide, N-methylacetamide, N,N-dimethylacetamide, and N-methylpyrrolidone; and lactones such as γ-lactone. These solvents can be used alone or in combination of two or more kinds.

The solvent used in the optical component forming composition of the present embodiment is preferably a safe solvent, more preferably at least one selected from PGMEA, PGME, CHN, CPN, 2-heptanone, anisole, butyl acetate, ethyl propionate, and ethyl lactate, and still more preferably at least one selected from PGMEA, PGME, and CHN.

In the optical component forming composition of the present embodiment, the relationship between the amount of the solid component and the amount of the solvent is not particularly limited, but preferably the solid component is 1 to 80% by mass and the solvent is 20 to 99% by mass, more preferably the solid component is 1 to 50% by mass and the solvent is 50 to 99% by mass, still more preferably the solid component is 2 to 40% by mass and the solvent is 60 to 98% by mass, and particularly preferably the solid component is 2 to 10% by mass and the solvent is 90 to 98% by mass, based on the total of the amount of the solid component and the solvent.

The optical component forming composition of the present embodiment may contain at least one selected from the group consisting of an acid generating agent (C), an acid crosslinking agent (G), an acid diffusion controlling agent (E), and a further component (F), as other solid components.

In the optical component forming composition of the present embodiment, the content of the compound represented by the formula (A-1) and the resin comprising a constitutional unit derived from the compound represented by the formula (A-1) (i.e., the tellurium-containing compound and the tellurium-containing resin) is not particularly limited, but is preferably 50 to 99.4% by mass of the total mass of the solid components (summation of the compound represented by the formula (A-1) and the resin comprising a constitutional unit derived from the compound represented by the formula (A-1), and optionally used solid components such as acid generating agent (C), acid crosslinking agent (G), acid diffusion controlling agent (E), and further component (F), hereinafter the same), more preferably 55 to 90% by mass, still more preferably 60 to 80% by mass, and particularly preferably 60 to 70% by mass.

When both the compound represented by the formula (A-1) and the resin comprising a constitutional unit derived from the compound represented by the formula (A-1) are contained, the content refers to the total amount of the compound represented by the formula (A-1) and the resin comprising a constitutional unit derived from the compound represented by the formula (A-1).

### (Acid Generating Agent (C))

The optical component forming composition of the present embodiment preferably contains one or more acid generating agents (C) generating an acid directly or indirectly by heat.

In this case, in the optical component forming composition of the present embodiment, the content of the acid generating agent (C) is preferably 0.001 to 49% by mass of the total mass of the solid components, more preferably 1 to 40% by mass, still more preferably 3 to 30% by mass, and particularly preferably 10 to 25% by mass. By using the acid generating agent (C) within the above content range, higher refractive index is obtained.

Concerning the optical component forming composition of the present embodiment, the acid generation method is not particularly limited as long as an acid is generated in the system. By using excimer laser instead of ultraviolet such as g-ray and i-ray, finer processing is possible, and also by using electron beam, extreme ultraviolet, X-ray or ion beam as a high energy ray, further finer processing is possible.

The acid generating agent (C) is not particularly limited, and is preferably at least one kind selected from the group consisting of compounds represented by the following formulae (8-1) to (8-8):

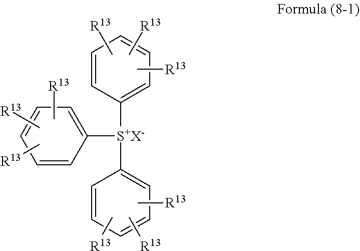

(In the formula (8-1), R¹³ may be each the same or different, and are each independently a hydrogen atom, a linear, branched or cyclic alkyl group, a linear, branched or cyclic alkoxy group, a hydroxyl group, or a halogen atom, X⁻ is an alkyl group, an aryl group, a sulfonic acid ion having a halogen substituted alkyl group or a halogen substituted aryl group, or a halide ion.)

The compound represented by the above formula (8-1) is preferably at least one kind selected from the group consisting of triphenylsulfonium trifluoromethanesulfonate, triphenylsulfonium nonafluoro-n-butanesulfonate, diphenyltolylsulfonium nonafluoro-n-butanesulfonate, triphenylsulfonium perfluoro-n-octanesulfonate, diphenyl-4-methylphenylsulfonium trifluoromethanesulfonate, di-2,4,6-trimethylphenylsulfonium trifluoromethanesulfonate, diphenyl-4-t-butoxyphenylsulfonium trifluoromethanesulfonate, diphenyl-4-t-butoxyphenylsulfonium nonafluoro-n-butanesulfonate, diphenyl-4-hydroxyphenylsulfonium trifluoromethanesulfonate, bis(4-fluorophenyl)-4-hydroxyphenylsulfonium trifluoromethanesulfonate, diphenyl-4-hydroxyphenylsulfonium nonafluoro-n-butanesulfonate, bis(4-hydroxyphenyl)-phenylsulfonium trifluoromethanesulfonate, tri(4-methoxyphenyl)sulfonium trifluoromethanesulfonate, tri(4-fluorophenyl)sulfonium trifluoromethanesulfonate, triphenylsulfonium p-toluenesulfonate, triphenylsulfonium benzenesulfonate, diphenyl-2,4,6-trimethylphenylsulfonium-p-toluenesulfonate, diphenyl-2,4,6-trimethylphenylsulfonium-2-trifluoromethylbenzenesulfonate, diphenyl-2,4,6-trimethylphenylsulfonium-4-trifluoromethylbenzenesulfonate, diphenyl-2,4,6-trimethylphenylsulfonium-2,4-difluorobenzenesulfonate, diphenyl-2,4,6-trimethylphenylsulfonium hexafluorobenzenesulfonate, diphenylnaphthylsulfonium trifluoromethanesulfonate, diphenyl-4-hydroxyphenylsulfonium-p-toluenesulfonate, triphenylsulfonium 10-camphorsulfonate, diphenyl-4-hydroxyphenylsulfonium 10-camphorsulfonate, and cyclo(1,3-perfluoropropanedisulfone)imidate.

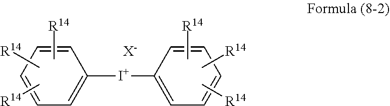

(In the formula (8-2), R¹⁴ may be each the same or different, and each independently represents a hydrogen atom, a linear, branched or cyclic alkyl group, a linear, branched or cyclic alkoxy group, a hydroxyl group, or a halogen atom. X⁻ is the same as above.)

The compound represented by the above formula (8-2) is preferably at least one kind selected from the group consisting of bis(4-t-butylphenyl)iodonium trifluoromethanesulfonate, bis(4-t-butylphenyl)iodonium nonafluoro-n-butanesulfonate, bis(4-t-butylphenyl)iodonium perfluoro-n-octanesulfonate, bis(4-t-butylphenyl)iodonium p-toluenesulfonate, bis(4-t-butylphenyl)iodonium benzenesulfonate, bis(4-t-butylphenyl)iodonium-2-trifluoromethylbenzenesulfonate, bis(4-t-butylphenyl)iodonium-4-trifluoromethylbenzenesulfonate, bis(4-t-butylphenyl)iodonium-2,4-difluorobenzenesulfonate, bis(4-t-butylphenyl)iodonium hexafluorobenzenesulfonate, bis(4-t-butylphenyl)iodonium 10-camphorsulfonate, diphenyliodonium trifluoromethanesulfonate, diphenyliodonium nonafluoro-n-butanesulfonate, diphenyliodonium perfluoro-n-octanesulfonate, diphenyliodonium p-toluenesulfonate, diphenyliodonium benzenesulfonate, diphenyliodonium 10-camphorsulfonate, diphenyliodonium-2-trifluoromethylbenzenesulfonate, diphenyliodonium-4-trifluoromethylbenzenesulfonate, diphenyliodonium-2,4-difluorobenzenesulfonate, diphenyliodonium hexafluorobenzenesulfonate, di(4-trifluoromethylphenyl)iodonium trifluoromethanesulfonate, di(4-trifluoromethylphenyl)iodonium nonafluoro-n-butanesulfonate, di(4-trifluoromethylphenyl)iodonium perfluoro-n-octanesulfonate, di(4-trifluoromethylphenyl)iodonium p-toluenesulfonate, di(4-trifluoromethylphenyl)iodonium benzenesulfonate, and di(4-trifluoromethylphenyl)iodonium 10-camphersulfonate.

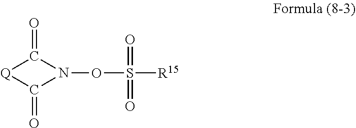

(In the formula (8-3), Q is an alkylene group, an arylene group, or an alkoxylene group, and R¹⁵ is an alkyl group, an aryl group, a halogen substituted alkyl group, or a halogen substituted aryl group.)

The compound represented by the above formula (8-3) is preferably at least one kind selected from the group consisting of N-(trifluoromethylsulfonyloxy)succinimide, N-(trifluoromethylsulfonyloxy)phthalimide, N-(trifluoromethylsulfonyloxy)diphenylmaleimide, N-(trifluoromethylsulfonyloxy)bicyclo[2.2.1]hept-5-en-2,3-dicarboxyimide, N-(trifluoromethylsulfonyloxy)naphthylimide, N-(10-camphorsulfonyloxy)succinimide, N-(10-camphorsulfonyloxy)phthalimide, N-(10-camphorsulfonyloxy)diphenylmaleimide, N-(10-camphorsulfonyloxy)bicyclo[2.2.1]hept-5-en-2,3-dicarboxyimide, N-(10-camphorsulfonyloxy)naphthylimide, N-(n-octanesulfonyloxy)bicyclo[2.2.1]hept-5-en-2,3-dicarboxyimide, N-(n-octanesulfonyloxy)naphthylimide, N-(p-toluenesulfonyloxy)bicyclo[2.2.1]hept-5-en-2,3-dicarboxyimide, N-(p-toluenesulfonyloxy)naphthylimide, N-(2-trifluoromethylbenzenesulfonyloxy)bicyclo[2.2.1]hept-5-en-2,3-dicarboxyimide, N-(2-trifluoromethylbenzenesulfonyloxy)naphthylimide, N-(4-trifluoromethylbenzenesulfonyloxy)bicyclo[2.2.1]hept-5-en-2,3-dicarboxyimide, N-(4-trifluoromethylbenzenesulfonyloxy)naphthylimide, N-(perfluorobenzenesulfonyloxy)bicyclo[2.2.1]hept-5-en-2,3-dicarboxyimide, N-(perfluorobenzenesulfonyloxy)naphthylimide, N-(1-naphthalenesulfonyloxy)bicyclo[2.2.1]hept-5-en-2,3-dicarboxyimide, N-(1-naphthalenesulfonyloxy)naphthylimide, N-(nonafluoro-n-butanesulfonyloxy)bicyclo[2.2.1]hept-5-en-2,3-dicarboxyimide, N-(nonafluoro-n-butanesulfonyloxy)naphthylimide, N-(perfluoro-n-octanesulfonyloxy)bicyclo[2.2.1]hept-5-en-2,3-dicarboxyimide, and N-(perfluoro-n-octanesulfonyloxy)naphthylimide.

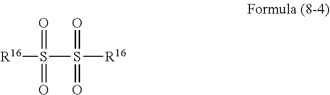

(In the formula (8-4), R¹⁶ may be each the same or different, and are each independently an optionally substituted linear, branched or cyclic alkyl group, an optionally substituted aryl group, an optionally substituted heteroaryl group, or an optionally substituted aralkyl group.)

The compound represented by the above formula (8-4) is preferably at least one kind selected from the group consisting of diphenyldisulfone, di(4-methylphenyl)disulfone, dinaphthyldisulfone, di(4-tert-butylphenyl)disulfone, di(4-hydroxyphenyl)disulfone, di(3-hydroxynaphthyl)disulfone, di(4-fluorophenyl)disulfone, di(2-fluorophenyl)disulfone, and di(4-trifluoromethylphenyl)disulfone.

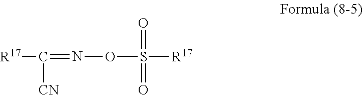

(In the formula (8-5), R¹⁷ may be the same or different, and are each independently an optionally substituted linear, branched or cyclic alkyl group, an optionally substituted aryl group, an optionally substituted heteroaryl group, or an optionally substituted aralkyl group.)

The compound represented by the above formula (8-5) is preferably at least one kind selected from the group consisting of α-(methylsulfonyloxyimino)-phenylacetonitrile, α-(methylsulfonyloxyimino)-4-methoxyphenylacetonitrile, α-(trifluoromethylsulfonyloxyimino)-phenylacetonitrile, α-(trifluoromethylsulfonyloxyimino)-4-methoxyphenylacetonitrile, α-(ethylsulfonyloxyimino)-4-methoxyphenylacetonitrile, α-(propylsulfonyloxyimino)-4-methylphenylacetonitrile, and α-(methylsulfonyloxyimino)-4-bromophenylacetonitrile.

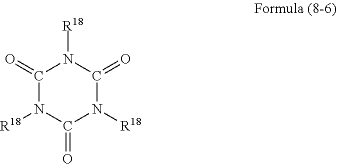

In the formula (8-6), R¹⁸ may be each the same or different, and are each independently a halogenated alkyl group having one or more chlorine atoms and one or more bromine atoms. The number of carbons in the halogenated alkyl group is preferably 1 to 5.

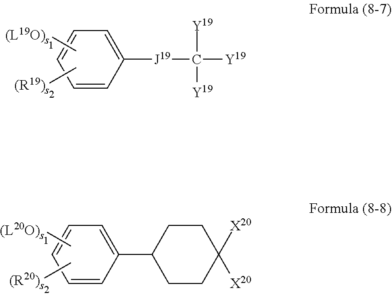

In the formulae (8-7) and (8-8), R¹⁹ and R²⁰ are each independently an alkyl group of 1 to 3 carbon atoms such as a methyl group, an ethyl group, an n-propyl group, and an isopropyl group; a cycloalkyl group such as a cyclopentyl group and a cyclohexyl group; an alkoxyl group of 1 to 3 carbon atoms such as a methoxy group, an ethoxy group, and a propoxy group; or an aryl group such as a phenyl group, a toluoyl group, and a naphthyl group, and preferably an aryl group of 6 to 10 carbon atom. L¹⁹ and L²⁰ are each independently an organic group having a 1,2-naphthoquinonediazide group. Specifically, preferable examples of the organic group having a 1,2-naphthoquinonediazide group include a 1,2-quinonediazidesulfonyl group such as a 1,2-naphthoquinonediazide-4-sulfonyl group, a 1,2-naphthoquinonediazide-5-sulfonyl group, and a 1,2-naphthoquinonediazide-6-sulfonyl group. Particularly, a 1,2-naphthoquinonediazide-4-sulfonyl group and a 1,2-naphthoquinonediazide-5-sulfonyl group are preferable. Each s₁ is independently an integer of 1 to 3; each s₂ is independently an integer of 0 to 4; and 1<s₁+s₂ 5. J¹⁹ is a single bond, a polymethylene group of 1 to 4 carbon atoms, a cycloalkylene group, a phenylene group, a group represented by the following formula (8-7-1), a carbonyl group, an ester group, an amide group, or an ether group. Y¹⁹ is a hydrogen atom, an alkyl group, or an aryl group, and X²⁰ are each independently a group represented by the following formula (8-8-1):

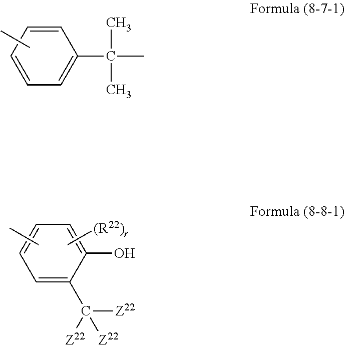

(In the above formula (8-8-1), Z²² are each independently an alkyl group, a cycloalkyl group, or an aryl group; R²² is an alkyl group, a cycloalkyl group, or an alkoxyl group; and r is an integer of 0 to 3.)

Examples of the other acid generating agent include bissulfonyldiazomethanes such as bis(p-toluenesulfonyl)diazomethane, bis(2,4-dimethylphenylsulfonyl)diazomethane, bis(tert-butylsulfonyl)diazomethane, bis(n-butylsulfonyl)diazomethane, bis(isobutylsulfonyl)diazomethane, bis(isopropylsulfonyl)diazomethane, bis(n-propylsulfonyl)diazomethane, bis(cyclohexylsulfonyl)diazomethane, bis(isopropylsulfonyl)diazomethane, 1,3-bis(cyclohexylsulfonylazomethylsulfonyl)propane, 1,4-bis(phenylsulfonylazomethylsulfonyl)butane, 1,6-bis(phenylsulfonylazomethylsulfonyl)hexane, and 1,10-bis(cyclohexylsulfonylazomethylsulfonyl)decane; and halogen-containing triazine derivatives such as 2-(4-methoxyphenyl)-4,6-(bistrichloromethyl)-1,3,5-triazine, 2-(4-methoxynaphthyl)-4,6-(bistrichloromethyl)-1,3,5-triazine, tris(2,3-dibromopropyl)-1,3,5-triazine, and tris(2,3-dibromopropyl)isocyanurate.

Among the acid generating agents, the acid generating agent (C) used in the optical component forming composition of the present embodiment is preferably an acid generating agent having an aromatic ring, and more preferably an acid generating agent represented by the formula (8-1) or (8-2). An acid generating agent having a sulfonate ion wherein X⁻ of the formula (8-1) or (8-2) has an aryl group or a halogen substituted aryl group is further preferable; an acid generating agent having a sulfonate ion wherein X⁻ of the formula (8-1) or (8-2) has an aryl group is particularly preferable; and diphenyltrimethylphenylsulfonium p-toluenesulfonate, triphenylsulfonium p-toluenesulfonate, triphenylsulfonium trifluoromethanesulfonate, and triphenylsulfonium nonafluoromethanesulfonate are particularly preferable. By using the acid generating agent, line edge roughness can be reduced.

The acid generating agent (C) can be used alone or in combination of two or more kinds.

### (Acid Crosslinking Agent (G))

The optical component forming composition of the present embodiment preferably contains one or more acid crosslinking agents (G), when used as an additive agent for enhancing the strength of a structure. The acid crosslinking agent (G) is a compound capable of intramolecular or intermolecular crosslinking the compound represented by the above formula (A-1) in the presence of the acid generated from the acid generating agent (C). Examples of such an acid crosslinking agent (G) include, but not particularly limited to, a compound having one or more groups (hereinafter, referred to as “crosslinkable group”) capable of crosslinking the compound represented by the above formula (A-1).

Specific examples of such a crosslinkable group are not particularly limited, and examples include (i) a hydroxyalkyl group or a group derived therefrom, such as a hydroxy (alkyl of 1 to 6 carbon atoms) group, an alkoxy of 1 to 6 carbon atoms (alkyl of 1 to 6 carbon atoms) group, and an acetoxy (alkyl of 1 to 6 carbon atoms) group; (ii) a carbonyl group or a group derived therefrom, such as a formyl group and a carboxy (alkyl of 1 to 6 carbon atoms) group; (iii) a nitrogenous group-containing group such as a dimethylaminomethyl group, a diethylaminomethyl group, a dimethylolaminomethyl group, a diethylolaminomethyl group, and a morpholinomethyl group; (iv) a glycidyl group-containing group such as a glycidyl ether group, a glycidyl ester group, and a glycidylamino group; (v) a group derived from an aromatic group such as an allyloxy of 1 to 6 carbon atoms (alkyl of 1 to 6 carbon atoms) group and an aralkyloxy of 1 to 6 carbon atoms (alkyl of 1 to 6 carbon atoms) group such as a benzyloxymethyl group and a benzoyloxymethyl group; and (vi) a polymerizable multiple bond-containing group such as a vinyl group and an isopropenyl group. As the crosslinkable group of the acid crosslinking agent (G), a hydroxyalkyl group and an alkoxyalkyl group or the like are preferable, and an alkoxymethyl group is particularly preferable.

Examples of the acid crosslinking agent (G) having the above crosslinkable group include, but not particularly limited to, (i) a methylol group-containing compound such as a methylol group-containing melamine compound, a methylol group-containing benzoguanamine compound, a methylol group-containing urea compound, a methylol group-containing glycoluryl compound, and a methylol group-containing phenolic compound; (ii) an alkoxyalkyl group-containing compound such as an alkoxyalkyl group-containing melamine compound, an alkoxyalkyl group-containing benzoguanamine compound, an alkoxyalkyl group-containing urea compound, an alkoxyalkyl group-containing glycoluryl compound, and an alkoxyalkyl group-containing phenolic compound; (iii) a carboxymethyl group-containing compound such as a carboxymethyl group-containing melamine compound, a carboxymethyl group-containing benzoguanamine compound, a carboxymethyl group-containing urea compound, a carboxymethyl group-containing glycoluryl compound, and a carboxymethyl group-containing phenolic compound; (iv) an epoxy compound such as a bisphenol A based epoxy compound, a bisphenol F based epoxy compound, a bisphenol S based epoxy compound, a novolac resin based epoxy compound, a resol resin based epoxy compound, and a poly(hydroxystyrene) based epoxy compound.

As the acid crosslinking agent (G), a compound having a phenolic hydroxyl group, and a compound and resin where the above crosslinkable group is introduced into an acid functional group in an alkali soluble resin to impart crosslinkability can be further used. The introduction rate of the crosslinkable group in that case is not particularly limited, and is adjusted to be, for example, 5 to 100 mol %, preferably 10 to 60 mol %, and more preferably 15 to 40 mol % based on the total acid functional groups in the compound having a phenolic hydroxy group, and the alkali soluble resin. Within the above range, the crosslinking reaction occurs sufficiently, and a decrease in the film remaining rate, and swelling phenomena and meandering or the like of a pattern are avoided, which is preferable.

In the optical component forming composition of the present embodiment, as the acid crosslinking agent (G), an alkoxyalkylated urea compound or resin thereof, or an alkoxyalkylated glycoluryl compound or resin thereof is preferable. Particularly preferable examples of the acid crosslinking agent (G) include compounds represented by the following formulae (11-1) to (11-3) and an alkoxymethylated melamine compound (acid crosslinking agent (G1)).

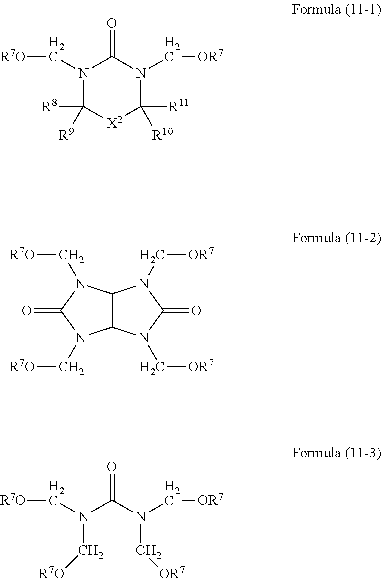

(In the above formulae (11-1) to (11-3), R⁷ each independently represents a hydrogen atom, an alkyl group, or an acyl group; R⁸ to R¹¹ each independently represents a hydrogen atom, a hydroxyl group, an alkyl group, or an alkoxyl group; and X² represents a single bond, a methylene group, or an oxygen atom.)

The alkyl group represented by R⁷ is not particularly limited, and is preferably of 1 to 6 carbon atoms, and more preferably of 1 to 3 carbon atoms. Examples thereof include a methyl group, an ethyl group, and a propyl group. The acyl group represented by R⁷ is not particularly limited, and is preferably of 2 to 6 carbon atoms, and more preferably of 2 to 4 carbon atoms. Examples thereof include an acetyl group and a propionyl group. The alkyl group represented by R⁸ to R¹¹ is not particularly limited, and is preferably of 1 to 6 carbon atoms, and more preferably of 1 to 3 carbon atoms. Examples thereof include a methyl group, an ethyl group, and a propyl group. The alkoxy group represented by R⁸ to R¹¹ is not particularly limited, and is preferably of 1 to 6 carbon atoms, and more preferably of 1 to 3 carbon atoms. Examples thereof include a methoxy group, an ethoxy group, and a propoxy group. X² is preferably a single bond or a methylene group. R⁷ to R¹¹ and X² may be substituted with an alkyl group such as a methyl group and an ethyl group, an alkoxy group such as a methoxy group and an ethoxy group, a hydroxyl group, and a halogen atom or the like. A plurality of R⁷ and R⁸ to R¹¹ may be each the same or different.

Specific examples of the compound represented by the formula (11-1) include compounds represented below.

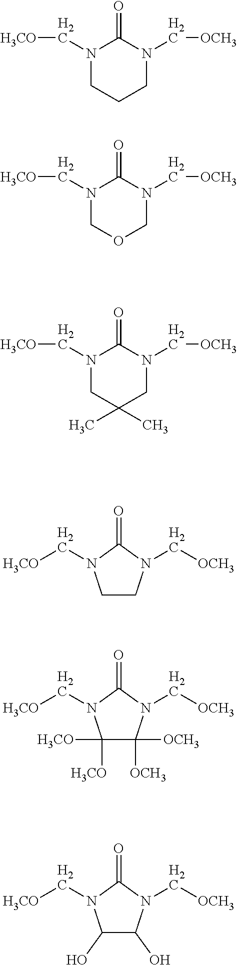

The compound represented by the formula (11-2) is not particularly limited, and specific examples include N,N,N,N-tetra(methoxymethyl)glycoluryl, N,N,N,N-tetra(ethoxymethyl)glycoluryl, N,N,N,N-tetra(n-propoxymethyl)glycoluryl, N,N,N,N-tetra(isopropoxymethyl)glycoluryl, N,N,N,N-tetra(n-butoxymethyl)glycoluryl, and N,N,N,N-tetra(t-butoxymethyl)glycoluryl. Among these, N,N,N,N-tetra(methoxymethyl)glycoluryl is particularly preferable.

The compound represented by the formula (11-3) is not particularly limited, and specific examples include compounds represented below.

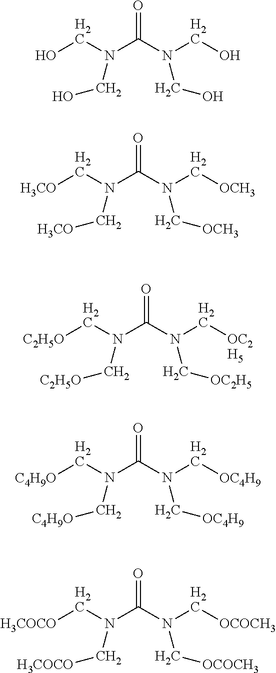

The alkoxymethylated melamine compound is not particularly limited, and specific examples include N,N,N,N,N,N-hexa(methoxymethyl)melamine, N,N,N,N,N,N-hexa(ethoxymethyl)melamine, N,N,N,N,N,N-hexa(n-propoxymethyl)melamine, N,N,N,N,N,N-hexa(isopropoxymethyl)melamine, N,N,N,N,N,N-hexa(n-butoxymethyl)melamine, and N,N,N,N,N,N-hexa(t-butoxymethyl)melamine. Among these, N,N,N,N,N,N-hexa(methoxymethyl)melamine is particularly preferable.

The above acid crosslinking agent (GI) can be obtained by, for example, conducting a condensation reaction of a urea compound or a glycoluryl compound with formalin to introduce a methylol group, etherifying the product with lower alcohols such as methyl alcohol, ethyl alcohol, propyl alcohol, and butyl alcohol, and then cooling the reaction solution to collect a precipitated compound or resin thereof. The above acid crosslinking agent (GI) can be obtained as a commercially available product such as CYMEL (trade name, manufactured by MT AquaPolymer) and NIKALAC (manufactured by Sanwa Chemical).

Other particularly preferable examples of the acid crosslinking agent (G) include a phenol derivative having 1 to 6 benzene rings within a molecule and two or more hydroxyalkyl groups and/or alkoxyalkyl groups within the entire molecule, the hydroxyalkyl groups and/or alkoxyalkyl groups being bonded to any of the above benzene rings (acid crosslinking agent (G2)). Preferable examples thereof include a phenol derivative having a molecular weight of 1500 or less, 1 to 6 benzene rings and a total of two or more hydroxyalkyl groups and/or alkoxyalkyl groups within a molecule, the hydroxyalkyl groups and/or alkoxyalkyl groups being bonded to any one of the above benzene rings, or a plurality of benzene rings.

The hydroxyalkyl group bonded to a benzene ring is not particularly limited to, and is the one of 1 to 6 carbon atoms such as a hydroxymethyl group, a 2-hydroxyethyl group, and a 2-hydroxy-1-propyl group is preferable. As the alkoxyalkyl group bonded to a benzene ring, the one of 2 to 6 carbon atoms is preferable. Specifically, a methoxymethyl group, an ethoxymethyl group, an n-propoxymethyl group, an isopropoxymethyl group, an n-butoxymethyl group, an isobutoxymethyl group, a sec-butoxymethyl group, a t-butoxymethyl group, a 2-methoxyethyl group, or a 2-methoxy-1-propyl group is preferable.

Among these phenol derivatives, particularly preferable ones are shown below:

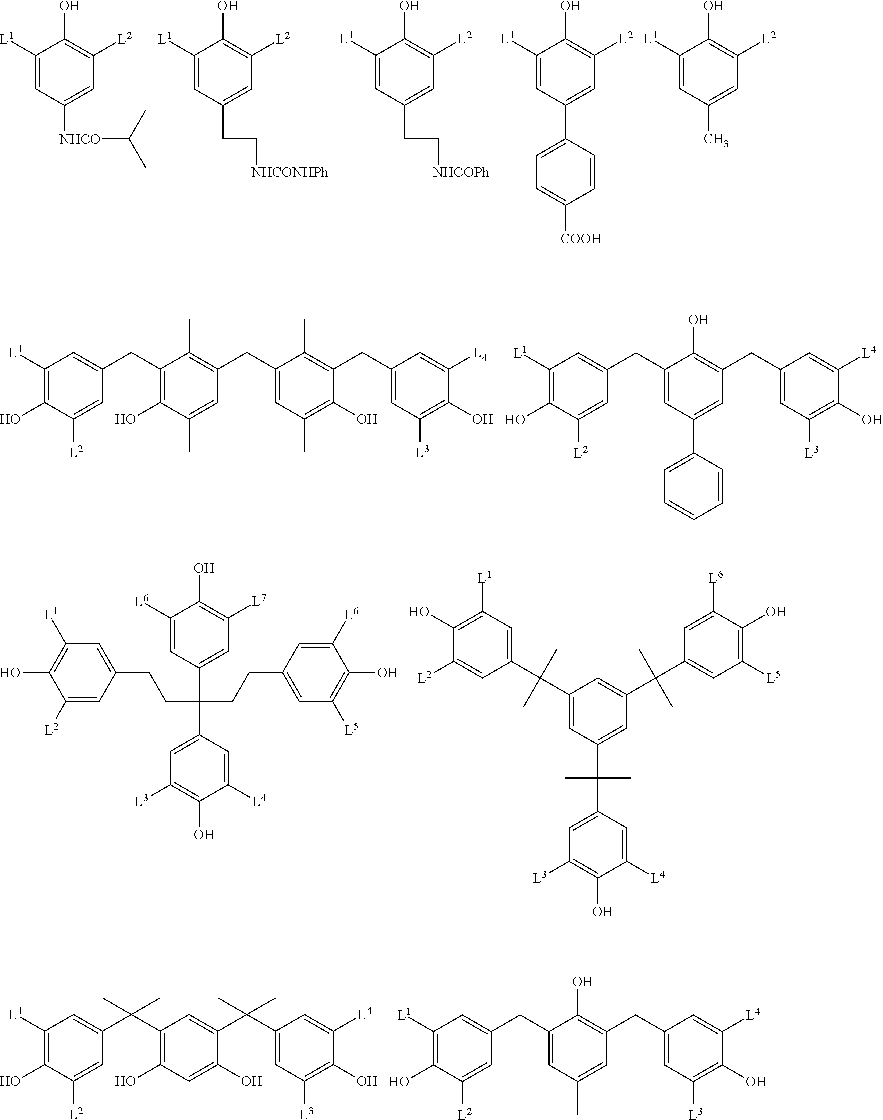

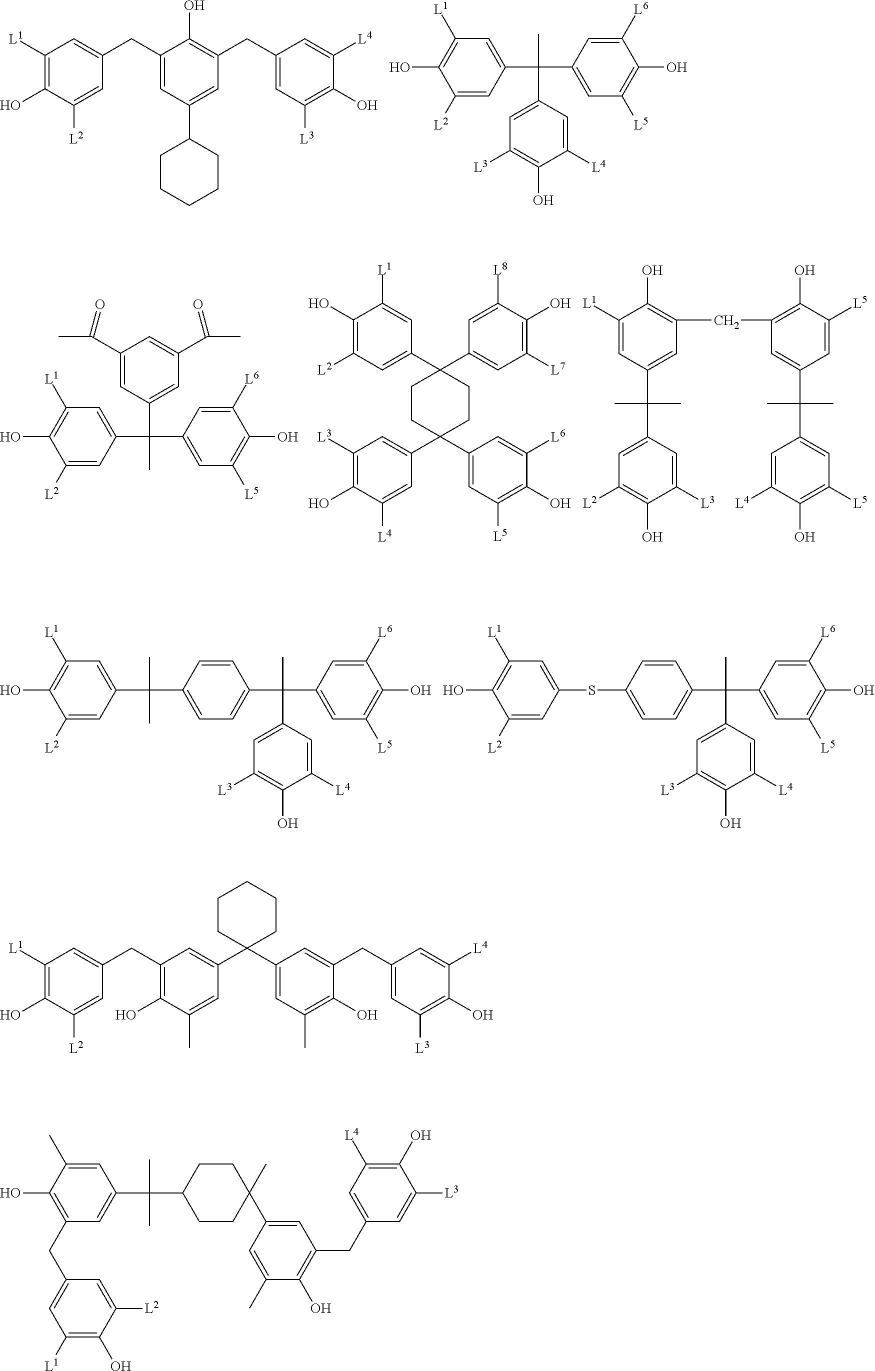

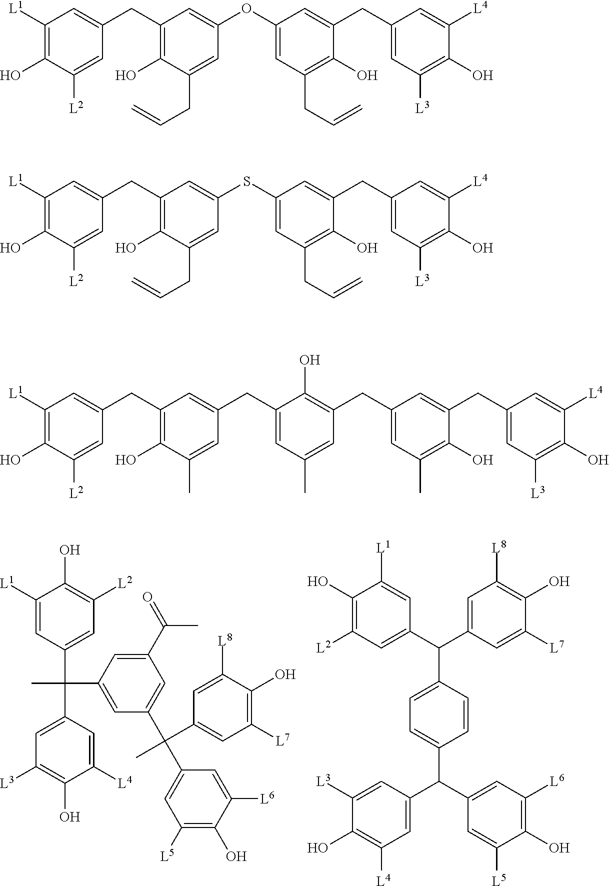

In the above formulae, L¹ to L⁸ may be the same or different, and each independently represents a hydroxymethyl group, a methoxymethyl group, or an ethoxymethyl group. A phenol derivative having a hydroxymethyl group can be obtained by reacting the corresponding phenolic compound having no hydroxymethyl group (a compound where L¹ to L⁸ in the above formulae are a hydrogen atom) with formaldehyde in the presence of a basic catalyst. In this case, in order to prevent resinification and gelation, the reaction temperature is preferably 60° C. or less. Specifically, it can be synthesized by methods described in Japanese Patent Application Laid-Open Nos. 6-282067 and 7-64285 or the like.

A phenol derivative having an alkoxymethyl group can be obtained by reacting the corresponding phenol derivative having a hydroxymethyl group with an alcohol in the presence of an acid catalyst. In this case, in order to prevent resinification and gelation, the reaction temperature is preferably 100° C. or less. Specifically, it can be synthesized by methods described in EP632003A1 or the like.

While the phenol derivative having a hydroxymethyl group and/or an alkoxymethyl group thus synthesized is preferable in terms of stability upon storage, the phenol derivative having an alkoxymethyl group is particularly preferable in terms of stability upon storage. The acid crosslinking agent (G2) may be used alone, or may be used in combination of two or more kinds.

Other particularly preferable examples of the acid crosslinking agent (G) include a compound having at least one α-hydroxyisopropyl group (acid crosslinking agent (G3)). The compound is not particularly limited in the structure, as long as it has an α-hydroxyisopropyl group. A hydrogen atom of a hydroxyl group in the above α-hydroxyisopropyl group may be substituted with one or more acid dissociation reactive groups (R—COO— group, R—SO₂— group or the like, wherein R represents a substituent group selected from the group consisting of a linear hydrocarbon group of 1 to 12 carbon atoms, a cyclic hydrocarbon group of 3 to 12 carbon atoms, an alkoxy group of 1 to 12 carbon atoms, a 1-branched alkyl group of 3 to 12 carbon atoms, and an aromatic hydrocarbon group of 6 to 12 carbon atoms). Examples of a compound having the above α-hydroxyisopropyl group include one kind or two kinds or more of a substituted or non-substituted aromatic based compound, a diphenyl compound, a naphthalene compound, a furan compound or the like containing at least one α-hydroxyisopropyl group. Specific examples thereof include a compound represented by the following formula (12-1) (hereinafter, referred to as “benzene based compound (1)”), a compound represented by the following formula (12-2) (hereinafter, referred to as “diphenyl based compound (2)”), a compound represented by the following formula (12-3) (hereinafter, referred to as “naphthalene based compound (3)”), and a compound represented by the following formula (12-4) (hereinafter, referred to as “furan based compound (4)”).

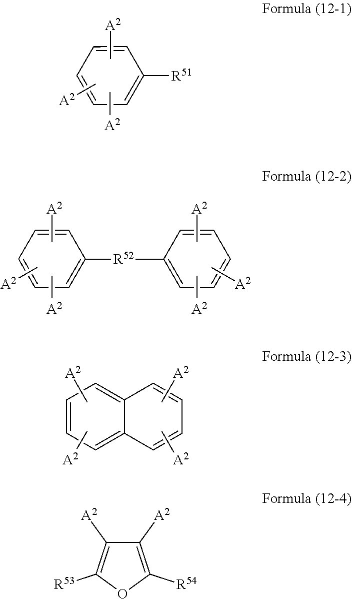

In the above formulae (12-1) to (12-4), each A² independently represents an α-hydroxyisopropyl group or a hydrogen atom, and at least one A² is an α-hydroxyisopropyl group.

In the formula (12-1), R⁵¹ represents a hydrogen atom, a hydroxyl group, a linear or branched alkylcarbonyl group of 2 to 6 carbon atoms, or a linear or branched alkoxycarbonyl group of 2 to 6 carbon atoms. Furthermore, in the formula (10-2), R⁵² represents a single bond, a linear or branched alkylene group of 1 to 5 carbon atoms, —O—, —CO—, or —COO—. Also, in the formula (12-4), R⁵³ and R⁵⁴ represent a hydrogen atom or a linear or branched alkyl group of 1 to 6 carbon atoms independently from each other.

Specific examples of the above benzene based compound (1) are not particularly limited, and examples include α-hydroxyisopropylbenzenes such as α-hydroxyisopropylbenzene, 1,3-bis(α-hydroxyisopropyl)benzene, 1,4-bis(α-hydroxyisopropyl)benzene, 1,2,4-tris(α-hydroxyisopropyl)benzene, and 1,3,5-tris(α-hydroxyisopropyl)benzene; α-hydroxyisopropylphenols such as 3-α-hydroxyisopropylphenol, 4-α-hydroxyisopropylphenol, 3,5-bis(α-hydroxyisopropyl)phenol, and 2,4,6-tris(α-hydroxyisopropyl)phenol; α-hydroxyisopropylphenyl alkyl ketones such as 3-α-hydroxyisopropylphenyl methyl ketone, 4-α-hydroxyisopropylphenyl methyl ketone, 4-α-hydroxyisopropylphenyl ethyl ketone, 4-α-hydroxyisopropylphenyl-n-propyl ketone, 4-α-hydroxyisopropylphenyl isopropyl ketone, 4-α-hydroxyisopropylphenyl-n-butyl ketone, 4-α-hydroxyisopropylphenyl-t-butyl ketone, 4-α-hydroxyisopropylphenyl-n-pentyl ketone, 3,5-bis(α-hydroxyisopropyl)phenyl methyl ketone, 3,5-bis(α-hydroxyisopropyl)phenyl ethyl ketone, and 2,4,6-tris(α-hydroxyisopropyl)phenyl methyl ketone; alkyl 4-α-hydroxyisopropylbenzoates such as methyl 3-α-hydroxyisopropylbenzoate, methyl 4-α-hydroxyisopropylbenzoate, ethyl 4-α-hydroxyisopropylbenzoate, n-propyl 4-α-hydroxyisopropylbenzoate, isopropyl 4-α-hydroxyisopropylbenzoate, n-butyl 4-α-hydroxyisopropylbenzoate, t-butyl 4-α-hydroxyisopropylbenzoate, n-pentyl 4-α-hydroxyisopropylbenzoate, methyl 3,5-bis(α-hydroxyisopropyl)benzoate, ethyl 3,5-bis(α-hydroxyisopropyl)benzoate, and methyl 2,4,6-tris(α-hydroxyisopropyl)benzoate.

Specific examples of the above diphenyl based compound (2) are not particularly limited, and examples include α-hydroxyisopropylbiphenyls such as 3-α-hydroxyisopropylbiphenyl, 4-α-hydroxyisopropylbiphenyl, 3,5-bis(α-hydroxyisopropyl)biphenyl, 3,3′-bis(α-hydroxyisopropyl)biphenyl, 3,4′-bis(α-hydroxyisopropyl)biphenyl, 4,4′-bis(α-hydroxyisopropyl)biphenyl, 2,4,6-tris(α-hydroxyisopropyl)biphenyl, 3,3′,5-tris(α-hydroxyisopropyl)biphenyl, 3,4′,5-tris(α-hydroxyisopropyl)biphenyl, 2,3′,4,6,-tetrakis(α-hydroxyisopropyl)biphenyl, 2,4,4′,6,-tetrakis(α-hydroxyisopropyl)biphenyl, 3,3′,5,5′-tetrakis(α-hydroxyisopropyl)biphenyl, 2,3′,4,5′,6-pentakis(α-hydroxyisopropyl)biphenyl, and 2,2′,4,4′,6,6′-hexakis(α-hydroxyisopropyl)biphenyl; α-hydroxyisopropyldiphenylalkanes such as 3-α-hydroxyisopropyldiphenylmethane, 4-α-hydroxyisopropyldiphenylmethane, 1-(4-α-hydroxyisopropylphenyl)-2-phenylethane, 1-(4-α-hydroxyisopropylphenyl)-2-phenylpropane, 2-(4-α-hydroxyisopropylphenyl)-2-phenylpropane, 1-(4-α-hydroxyisopropylphenyl)-3-phenylpropane, 1-(4-α-hydroxyisopropylphenyl)-4-phenylbutane, 1-(4-α-hydroxyisopropylphenyl)-5-phenylpentane, 3,5-bis(α-hydroxyisopropyldiphenylmethane, 3,3′-bis(α-hydroxyisopropyl)diphenylmethane, 3,4′-bis(α-hydroxyisopropyl)diphenylmethane, 4,4′-bis(α-hydroxyisopropyl)diphenylmethane, 1,2-bis(4-α-hydroxyisopropylphenyl)ethane, 1,2-bis(4-α-hydroxypropylphenyl)propane, 2,2-bis(4-α-hydroxypropylphenyl)propane, 1,3-bis(4-α-hydroxypropylphenyl)propane, 2,4,6-tris(α-hydroxyisopropyl)diphenylmethane, 3,3′,5-tris(α-hydroxyisopropyl)diphenylmethane, 3,4′,5-tris(α-hydroxyisopropyl)diphenylmethane, 2,3′,4,6-tetrakis(α-hydroxyisopropyl)diphenylmethane, 2,4,4′,6-tetrakis(α-hydroxyisopropyl)diphenylmethane, 3,3′,5,5′-tetrakis(α-hydroxyisopropyl)diphenylmethane, 2,3′,4,5′,6-pentakis(α-hydroxyisopropyl)diphenylmethane, and 2,2′,4,4′,6,6′-hexakis(α-hydroxyisopropyl)diphenylmethane; α-hydroxyisopropyldiphenyl ethers such as 3-α-hydroxyisopropyldiphenyl ether, 4-α-hydroxyisopropyldiphenyl ether, 3,5-bis(α-hydroxyisopropyl)diphenyl ether, 3,3′-bis(α-hydroxyisopropyl)diphenyl ether, 3,4′-bis(α-hydroxyisopropyl)diphenyl ether, 4,4′-bis(α-hydroxyisopropyl)diphenyl ether, 2,4,6-tris(α-hydroxyisopropyl)diphenyl ether, 3,3′,5-tris(α-hydroxyisopropyl)diphenyl ether, 3,4′,5-tris(α-hydroxyisopropyl)diphenyl ether, 2,3′,4,6-tetrakis(α-hydroxyisopropyl)diphenyl ether, 2,4,4′,6-tetrakis(α-hydroxyisopropyl)diphenyl ether, 3,3′,5,5′-tetrakis(α-hydroxyisopropyl)diphenyl ether, 2,3′,4,5′,6-pentakis(α-hydroxyisopropyl)diphenyl ether, and 2,2′,4,4′,6,6′-hexakis(α-hydroxyisopropyl)diphenyl ether; α-hydroxyisopropyldiphenyl ketones such as 3-α-hydroxyisopropyldiphenyl ketone, 4-α-hydroxyisopropyldiphenyl ketone, 3,5-bis(α-hydroxyisopropyl)diphenyl ketone, 3,3′-bis(α-hydroxyisopropyl)diphenyl ketone, 3,4′-bis(α-hydroxyisopropyl)diphenyl ketone, 4,4′-bis(α-hydroxyisopropyl)diphenyl ketone, 2,4,6-tris(α-hydroxyisopropyl)diphenyl ketone, 3,3′,5-tris(α-hydroxyisopropyl)diphenyl ketone, 3,4′,5-tris(α-hydroxyisopropyl)diphenyl ketone, 2,3′,4,6-tetrakis(α-hydroxyisopropyl)diphenyl ketone, 2,4,4′,6-tetrakis(α-hydroxyisopropyl)diphenyl ketone, 3,3′,5,5′-tetrakis(α-hydroxyisopropyl)diphenyl ketone, 2,3′,4,5′,6-pentakis(α-hydroxyisopropyl)diphenyl ketone, and 2,2′,4,4′,6,6′-hexakis(α-hydroxyisopropyl)diphenyl ketone; phenyl α-hydroxyisopropylbenzoates such as phenyl 3-α-hydroxyisopropylbenzoate, phenyl 4-α-hydroxyisopropylbenzoate, 3-α-hydroxyisopropylphenyl benzoate, 4-α-hydroxyisopropylphenyl benzoate, phenyl 3,5-bis(α-hydroxyisopropyl)benzoate, 3-α-hydroxyisopropylphenyl 3-α-hydroxyisopropylbenzoate, 4-α-hydroxyisopropylphenyl 3-α-hydroxyisopropylbenzoate, 3-α-hydroxyisopropylphenyl 4-α-hydroxyisopropylbenzoate, 4-α-hydroxyisopropylphenyl 4-α-hydroxyisopropylbenzoate, 3,5-bis(α-hydroxyisopropyl)phenyl benzoate, phenyl 2,4,6-tris(α-hydroxyisopropyl)benzoate, 3-α-hydroxyisopropylphenyl 3,5-bis(α-hydroxyisopropyl)benzoate, 4-α-hydroxyisopropylphenyl 3,5-bis(α-hydroxyisopropyl)benzoate, 3,5-bis(α-hydroxyisopropyl)phenyl 3-α-hydroxyisopropylbenzoate, 3,5-bis(α-hydroxyisopropyl)phenyl 4-α-hydroxyisopropylbenzoate, 2,4,6-tris(α-hydroxyisopropyl)phenyl benzoate, 3-α-hydroxyisopropylphenyl 2,4,6-tris(α-hydroxyisopropyl)benzoate, 4-α-hydroxyisopropylphenyl 2,4,6-tris(α-hydroxyisopropyl)benzoate, 3,5-bis(α-hydroxyisopropyl)phenyl 3,5-bis(α-hydroxyisopropyl)benzoate, 2,4,6-tris(α-hydroxyisopropyl)phenyl 3-α-hydroxyisopropylbenzoate, 2,4,6-tris(α-hydroxyisopropyl)phenyl 4-α-hydroxyisopropylbenzoate, 3,5-bis(α-hydroxyisopropyl)phenyl 2,4,6-tris(α-hydroxyisopropyl)benzoate, 2,4,6-tris(α-hydroxyisopropyl)phenyl 3,5-bis(α-hydroxyisopropyl)benzoate, and 2,4,6-tris(α-hydroxyisopropyl)phenyl 2,4,6-tris(α-hydroxyisopropyl)benzoate.

Furthermore, specific examples of the above naphthalene based compound (3) are not particularly limited, and examples include 1-(α-hydroxyisopropyl)naphthalene, 2-(α-hydroxyisopropyl)naphthalene, 1,3-bis(α-hydroxyisopropyl)naphthalene, 1,4-bis(α-hydroxyisopropyl)naphthalene, 1,5-bis(α-hydroxyisopropyl)naphthalene, 1,6-bis(α-hydroxyisopropyl)naphthalene, 1,7-bis(α-hydroxyisopropyl)naphthalene, 2,6-bis(α-hydroxyisopropyl)naphthalene, 2,7-bis(α-hydroxyisopropyl)naphthalene, 1,3,5-tris(α-hydroxyisopropyl)naphthalene, 1,3,6-tris(α-hydroxyisopropyl)naphthalene, 1,3,7-tris(α-hydroxyisopropyl)naphthalene, 1,4,6-tris(α-hydroxyisopropyl)naphthalene, 1,4,7-tris(α-hydroxyisopropyl)naphthalene, and 1,3,5,7-tetrakis(α-hydroxyisopropyl)naphthalene.

Specific examples of the above furan based compound (4) include, but not particularly limited to, 3-(α-hydroxyisopropyl)furan, 2-methyl-3-(α-hydroxyisopropyl)furan, 2-methyl-4-(α-hydroxyisopropyl)furan, 2-ethyl-4-(α-hydroxyisopropyl)furan, 2-n-propyl-4-(α-hydroxyisopropyl)furan, 2-isopropyl-4-(α-hydroxyisopropyl)furan, 2-n-butyl-4-(α-hydroxyisopropyl)furan, 2-t-butyl-4-(α-hydroxyisopropyl)furan, 2-n-pentyl-4-(α-hydroxyisopropyl)furan, 2,5-dimethyl-3-(α-hydroxyisopropyl)furan, 2,5-diethyl-3-(α-hydroxyisopropyl)furan, 3,4-bis(α-hydroxyisopropyl)furan, 2,5-dimethyl-3,4-bis(α-hydroxyisopropyl)furan, and 2,5-diethyl-3,4-bis(α-hydroxyisopropyl)furan.

As the above acid crosslinking agent (G3), a compound having two or more free α-hydroxyisopropyl groups is preferable; the above benzene based compound (1) having two or more α-hydroxyisopropyl groups, the above diphenyl based compound (2) having two or more α-hydroxyisopropyl groups, and the above naphthalene based compound (3) having two or more α-hydroxyisopropyl groups are further preferable; and α-hydroxyisopropylbiphenyls having two or more α-hydroxyisopropyl groups and the above naphthalene based compound (3) having two or more α-hydroxyisopropyl groups are particularly preferable.

The above acid crosslinking agent (G3) can normally be obtained by a method for reacting an acetyl group-containing compound such as 1,3-diacetylbenzene with Grignard reagent such as CH₃MgBr to methylate and then hydrolyzing, or a method for oxidizing an isopropyl group-containing compound such as 1,3-diisopropylbenzene with oxygen or the like to produce a peroxide and then reducing.

In the optical component forming composition of the present embodiment, the content of the acid crosslinking agent (G) is preferably 0.5 to 49% by mass of the total mass of the solid components, more preferably 0.5 to 40% by mass, still more preferably 1 to 30% by mass, and particularly preferably 2 to 20% by mass. When the content ratio of the above acid crosslinking agent (G) is 0.5% by mass or more, the inhibiting effect of the solubility of the optical component forming composition in an organic solvent can be improved, which is preferable. On the other hand, when the content is 49% by mass or less, a decrease in the heat resistance of the optical component forming composition can be inhibited, which is preferable.

The content of at least one kind of compound selected from the above acid crosslinking agent (G1), acid crosslinking agent (G2), and acid crosslinking agent (G3) in the above acid crosslinking agent (G) is also not particularly limited, and can be within various ranges according to the kind of substrates or the like used upon forming an optical component forming composition.

In all acid crosslinking agent components, the content of the alkoxymethylated melamine compound and/or the compounds represented by formula (12-1) to formula (12-3) is not particularly limited, but is preferably 50 to 99% by mass, more preferably 60 to 99% by mass, still more preferably 70 to 98% by mass, and particularly preferably 80 to 97% by mass. By having the alkoxymethylated melamine compound and/or the compounds represented by formula (12-1) to formula (12-3) of 50% by mass or more of all acid crosslinking agent components, the resolution can be further improved, which is preferable. By having the compounds of 99% by mass or less, the structure is likely to have a good shape, which is preferable.

### (Acid Diffusion Controlling Agent (E))

The optical component forming composition of the present embodiment may contain an acid diffusion controlling agent (E) having a function of controlling diffusion of an acid generated from an acid generating agent in the optical component forming composition to inhibit any unpreferable chemical reaction or the like. By using such an acid diffusion controlling agent (E), the storage stability of the optical component forming composition is improved. Also, along with the further improvement of the resolution, the line width change of a structure due to variation in the post exposure delay time after heating can be inhibited, and the composition has extremely excellent process stability.

Such an acid diffusion controlling agent (E) is not particularly limited, and examples include a radiation degradable basic compound such as a nitrogen atom-containing basic compound, a basic sulfonium compound, and a basic iodonium compound. The acid diffusion controlling agent (E) can be used alone or in combination of two or more kinds.

The above acid diffusion controlling agent is not particularly limited, and examples include a nitrogen-containing organic compound, and a basic compound degradable by exposure. The nitrogen-containing organic compound is not particularly limited, and examples include a compound represented by the following formula (14):

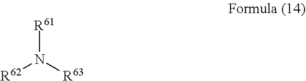

(hereinafter, referred to as a “nitrogen-containing compound (I)”), a diamino compound having two nitrogen atoms within the same molecule (hereinafter, referred to as a “nitrogen-containing compound (II)”), a polyamino compound or polymer having three or more nitrogen atoms (hereinafter, referred to as a “nitrogen-containing compound (III)”), an amide group-containing compound, a urea compound, and a nitrogen-containing heterocyclic compound. The acid diffusion controlling agent (E) may be used alone as one kind or may be used in combination of two or more kinds.

In the above formula (14), R⁶¹, R⁶², and R⁶³ represent a hydrogen atom, a linear, branched or cyclic alkyl group, an aryl group, or an aralkyl group independently from each other. The above alkyl group, aryl group, or aralkyl group may be non-substituted or may be substituted with a hydroxyl group or the like. Herein, the above linear, branched or cyclic alkyl group is not particularly limited, and examples include the one of 1 to 15 carbon atoms, and preferably 1 to 10 carbon atoms. Specific examples thereof include a methyl group, an ethyl group, an n-propyl group, an isopropyl group, an n-butyl group, an isobutyl group, a sec-butyl group, a t-butyl group, an n-pentyl group, a neopentyl group, an n-hexyl group, a texyl group, an n-heptyl group, an n-octyl group, an n-ethylhexyl group, an n-nonyl group, and an n-decyl group. Examples of the above aryl group include the one of 6 to 12 carbon atoms. Specific examples thereof include a phenyl group, a tolyl group, a xylyl group, a cumenyl group, and a 1-naphthyl group. Furthermore, the above aralkyl group is not particularly limited, and examples include the one of 7 to 19 carbon atoms, and preferably 7 to 13 carbon atoms. Specific examples thereof include a benzyl group, an α-methylbenzyl group, a phenethyl group, and a naphthylmethyl group.

The above nitrogen-containing compound (I) is not particularly limited, and specific examples include particularly mono(cyclo)alkylamines such as n-hexylamine, n-heptylamine, n-octylamine, n-nonylamine, n-decylamine, n-dodecylamine, and cyclohexylamine; di(cyclo)alkylamines such as di-n-butylamine, di-n-pentylamine, di-n-hexylamine, di-n-heptylamine, di-n-octylamine, di-n-nonylamine, di-n-decylamine, methyl-n-dodecylamine, di-n-dodecylmethyl, cyclohexylmethylamine, and dicyclohexylamine; tri(cyclo)alkylamines such as triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, dimethyl-n-dodecylamine, di-n-dodecylmethylamine, dicyclohexylmethylamine, and tricyclohexylamine; alkanolamines such as monoethanolamine, diethanolamine, and triethanolamine; and aromatic amines such as aniline, N-methylaniline, N,N-dimethylaniline, 2-methylaniline, 3-methylaniline, 4-methylaniline, 4-nitroaniline, diphenylamine, triphenylamine, and 1-naphthylamine.

The above nitrogen-containing compound (II) is not particularly limited, and specific examples include particularly ethylenediamine, N,N,N′,N′-tetramethylethylenediamine, N,N,N′,N′-tetrakis(2-hydroxypropyl)ethylenediamine, tetramethylenediamine, hexamethylenediamine, 4,4′-diaminodiphenylmethane, 4,4′-diaminodiphenyl ether, 4,4′-diaminobenzophenone, 4,4′-diaminodiphenylamine, 2,2-bis(4-aminophenyl)propane, 2-(3-aminophenyl)-2-(4-aminophenyl)propane, 2-(4-aminophenyl)-2-(3-hydroxyphenyl)propane, 2-(4-aminophenyl)-2-(4-hydroxyphenyl)propane, 1,4-bis[1-(4-aminophenyl)-1-methylethyl]benzene, and 1,3-bis[1-(4-aminophenyl)-1-methylethyl]benzene.

The above nitrogen-containing compound (III) is not particularly limited, and specific examples include particularly polymers of polyethyleneimine, polyarylamine, and N-(2-dimethylaminoethyl)acrylamide.

The above amide group-containing compound is not particularly limited, and specific examples include particularly formamide, N-methylformamide, N,N-dimethylformamide, acetamide, N-methylacetamide, N,N-dimethylacetamide, propioneamide, benzamide, pyrrolidone, and N-methylpyrrolidone.

The above urea compound is not particularly limited, and specific examples include particularly urea, methylurea, 1,1-dimethylurea, 1,3-dimethylurea, 1,1,3,3-tetramethylurea, 1,3-diphenylurea, and tri-n-butylthiourea.

The above nitrogen-containing heterocyclic compound is not particularly limited, and specific examples include particularly imidazoles such as imidazole, benzimidazole, 4-methylimidazole, 4-methyl-2-phenylimidazole, and 2-phenylbenzimidazole; pyridines such as pyridine, 2-methylpyridine, 4-methylpyridine, 2-ethylpyridine, 4-ethylpyridine, 2-phenylpyridine, 4-phenylpyridine, 2-methyl-4-phenylpyridine, nicotine, nicotinic acid, amide nicotinate, quinoline, 8-oxyquinoline, and acridine; and pyrazine, pyrazole, pyridazine, quinozaline, purine, pyrrolidine, piperidine, morpholine, 4-methylmorpholine, piperazine, 1,4-dimethylpiperazine, and 1,4-diazabicyclo[2.2.2]octane.

The radiation degradable basic compound is not particularly limited, and examples include a sulfonium compound represented by the following formula (15-1) and an iodonium compound represented by the following formula (15-2):

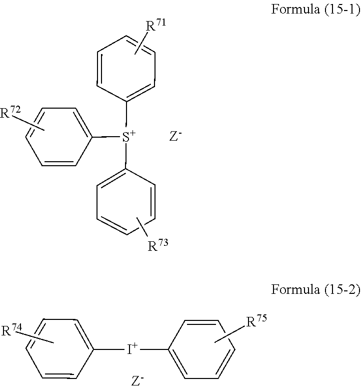

In the above formulae (15-1) and (15-2), R⁷¹, R⁷², R⁷³, R⁷⁴, and R⁷⁵ each independently represent a hydrogen atom, an alkyl group of 1 to 6 carbon atoms, an alkoxyl group of 1 to 6 carbon atoms, a hydroxyl group, or a halogen atom. Z⁻ represents HO—, R—COO— (R represents an alkyl group of 1 to 6 carbon atoms, an aryl group of 6 to 11 carbon atoms, or an alkaryl group of 7 to 12 carbon atoms), or an anion represented by the following formula (15-3):

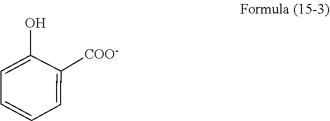

Specific examples of the above radiation degradable basic compound are not particularly limited, and examples include triphenylsulfonium hydroxide, triphenylsulfonium acetate, triphenylsulfonium salicylate, diphenyl-4-hydroxyphenylsulfonium hydroxide, diphenyl-4-hydroxyphenylsulfonium acetate, diphenyl-4-hydroxyphenylsulfonium salicylate, bis(4-t-butylphenyl)iodonium hydroxide, bis(4-t-butylphenyl)iodonium acetate, bis(4-t-butylphenyl)iodonium hydroxide, bis(4-t-butylphenyl)iodonium acetate, bis(4-t-butylphenyl)iodonium salicylate, 4-t-butylphenyl-4-hydroxyphenyliodonium hydroxide, 4-t-butylphenyl-4-hydroxyphenyliodonium acetate, and 4-t-butylphenyl-4-hydroxyphenyliodonium salicylate.

The content of the acid diffusion controlling agent (E) is preferably 0.001 to 49% by mass of the total mass of the solid component, more preferably 0.01 to 10% by mass, still more preferably 0.01 to 5% by mass, and particularly preferably 0.01 to 3% by mass. When the content of the acid diffusion controlling agent (E) is within the above range, a decrease in resolution, and deterioration of the pattern shape and the dimension fidelity or the like can be further inhibited. Moreover, even though the post exposure delay time from electron beam irradiation to heating after radiation irradiation becomes longer, the shape of the pattern upper layer portion does not deteriorate. When the content of the acid diffusion controlling agent (E) is 10% by mass or less, a decrease in sensitivity, and developability of the unexposed portion or the like can be prevented. By using such an acid diffusion controlling agent, the storage stability of an optical component forming composition improves, also along with improvement of the resolution, the line width change of an optical component forming composition due to variation in the post exposure delay time before radiation irradiation and the post exposure delay time after radiation irradiation can be inhibited, and the composition is extremely excellent process stability.

### (Other Optional Component (F))

To the optical component forming composition of the present embodiment, within the range of not inhibiting the purpose of the present embodiment, if required, as the other optional component (F), one kind or two kinds or more of various additive agents such as a dissolution promoting agent, a dissolution controlling agent, a sensitizing agent, a surfactant and an organic carboxylic acid or an oxo acid of phosphor, or derivative thereof can be added.

### —Dissolution Promoting Agent—

A low molecular weight dissolution promoting agent is a component having a function of increasing the solubility of the compound represented by the formula (A-1) or the resin comprising a constitutional unit derived from the compound represented by the formula (A-1) in a developing solution to moderately increase the dissolution rate of the compound upon developing, when the solubility of the compound or the resin is too low. The low molecular weight dissolution promoting agent can be used, within the range of not deteriorating the effect of the present invention. Examples of the above dissolution promoting agent can include low molecular weight phenolic compounds, such as bisphenols and tris(hydroxyphenyl)methane. These dissolution promoting agents can be used alone or in mixture of two or more kinds. The content of the dissolution promoting agent, which is arbitrarily adjusted according to the kind of the tellurium-containing compound represented by the formula (A-1) to be used, is preferably 0 to 49% by mass of the total mass of the solid component, more preferably 0 to 5% by mass, still more preferably 0 to 1% by mass, and particularly preferably 0% by mass.

### —Dissolution Controlling Agent—

The dissolution controlling agent is a component having a function of controlling the solubility of the compound represented by the formula (A-1) or the resin comprising a constitutional unit derived from the compound represented by the formula (A-1) in a developing solution to moderately decrease the dissolution rate upon developing, when the solubility of the compound or the resin is too high. As such a dissolution controlling agent, the one which does not chemically change in steps such as calcination of an optical component, heating, and development is preferable.

The dissolution controlling agent is not particularly limited, and examples include aromatic hydrocarbons such as phenanthrene, anthracene, and acenaphthene; ketones such as acetophenone, benzophenone, and phenyl naphtyl ketone; and sulfones such as methyl phenyl sulfone, diphenyl sulfone, and dinaphthyl sulfone. These dissolution controlling agents can be used alone or in two or more kinds.

The content of the dissolution controlling agent is not particularly limited and is arbitrarily adjusted according to the kind of the compound represented by the formula (A-1) or the resin comprising a constitutional unit derived from the compound represented by the formula (A-1) to be used, but is preferably 0 to 49% by mass of the total mass of the solid component, more preferably 0 to 5% by mass, still more preferably 0 to 1% by mass, and particularly preferably 0% by mass.

### —Sensitizing Agent—

The sensitizing agent is a component having a function of absorbing irradiated radiation energy, transmitting the energy to the acid generating agent (C), and thereby increasing the acid production amount, and improving the apparent sensitivity of a resist. Such a sensitizing agent is not particularly limited, and examples include benzophenones, biacetyls, pyrenes, phenothiazines, and fluorenes. These sensitizing agents can be used alone or in two or more kinds. The content of the sensitizing agent, which is arbitrarily adjusted according to the kind of the compound represented by the formula (A-1) or the resin comprising a constitutional unit derived from the compound represented by the formula (A-1) to be used, is preferably 0 to 49% by mass of the total mass of the solid component, more preferably 0 to 5% by mass, still more preferably 0 to 1% by mass, and particularly preferably 0% by mass.

### —Surfactant—

The surfactant is a component having a function of improving coatability and striation of the optical component forming composition of the present embodiment or the like. Such a surfactant is not particularly limited, and may be any of anionic, cationic, nonionic or amphoteric. A preferable surfactant is a nonionic surfactant. The nonionic surfactant has a good affinity with a solvent used in production of optical component forming compositions and more effects. Examples of the nonionic surfactant include, but not particularly limited to, a polyoxyethylene higher alkyl ethers, polyoxyethylene higher alkyl phenyl ethers, and higher fatty acid diesters of polyethylene glycol. Examples of commercially available products include, hereinafter by trade name, EFTOP (manufactured by Jemco Inc.), MEGAFAC (manufactured by DIC Corporation), Fluorad (manufactured by Sumitomo 3M Limited), AsahiGuard, Surflon (hereinbefore, manufactured by Asahi Glass Co., Ltd.), Pepole (manufactured by Toho Chemical Industry Co., Ltd.), KP (manufactured by Shin-Etsu Chemical Co., Ltd.), and Polyflow (manufactured by Kyoeisha Chemical Co., Ltd.). The content of the surfactant is not particularly limited, and is arbitrarily adjusted according to the kind of the compound represented by the formula (A-1) or the resin comprising a constitutional unit derived from the compound represented by the formula (A-1) to be used, but is preferably 0 to 49% by mass of the total mass of the solid component, more preferably 0 to 5% by mass, still more preferably 0 to 1% by mass, and particularly preferably 0% by mass.

### —Organic Carboxylic Acid or Oxo Acid of Phosphor or Derivative Thereof—

For the purpose of prevention of sensitivity deterioration or improvement of a structure and post exposure delay stability or the like, and as an additional optional component, the optical component forming composition of the present embodiment may contain an organic carboxylic acid or an oxo acid of phosphor or derivative thereof. The composition can be used in combination with the acid diffusion controlling agent, or may be used alone. The organic carboxylic acid is not particularly limited, and, for example, is suitably malonic acid, citric acid, malic acid, succinic acid, benzoic acid, salicylic acid, or the like. Examples of the oxo acid of phosphor or derivative thereof include phosphoric acid or derivative thereof such as ester including phosphoric acid, di-n-butyl ester phosphate, and diphenyl ester phosphate; phosphonic acid or derivative thereof such as ester including phosphonic acid, dimethyl ester phosphonate, di-n-butyl ester phosphonate, phenylphosphonic acid, diphenyl ester phosphonate, and dibenzyl ester phosphonate; and phosphinic acid and derivative thereof such as ester including phosphinic acid and phenylphosphinic acid. Among these, phosphonic acid is particularly preferable.

The organic carboxylic acid or the oxo acid of phosphor or derivative thereof can be used alone or in combination of two or more kinds. The content of the organic carboxylic acid or the oxo acid of phosphor or derivative thereof, which is arbitrarily adjusted according to the kind of the compound represented by the formula (A-1) or the resin comprising a constitutional unit derived from the compound represented by the formula (A-1) to be used, is preferably 0 to 49% by mass of the total mass of the solid component, more preferably 0 to 5% by mass, still more preferably 0 to 1% by mass, and particularly preferably 0% by mass.

### —Other Additive Agent—

Furthermore, the optical component forming composition of the present embodiment can contain one kind or two kinds or more of additive agents other than the above dissolution controlling agent, sensitizing agent, and surfactant, within the range of not inhibiting the purpose of the present invention, if required. Examples of such an additive agent include, but not particularly limited to, a dye, a pigment, and an adhesion aid. For example, the composition contains the dye or the pigment, and thereby a latent image of the exposed portion is visualized and influence of halation upon exposure can be alleviated, which is preferable. The composition contains the adhesion aid, and thereby adhesiveness to a substrate can be improved, which is preferable. Furthermore, examples of other additive agent can include, but not particularly limited to, a halation preventing agent, a storage stabilizing agent, a defoaming agent, and a shape improving agent. Specific examples thereof can include 4-hydroxy-4′-methylchalkone.

The total content of the optional component (F) is preferably 0 to 49% by mass of the total mass of the solid component, more preferably 0 to 5% by mass, still more preferably 0 to 1% by mass, and particularly preferably 0% by mass.

In the optical component forming composition of the present embodiment, the content of the compound represented by the formula (A-1) or the resin comprising a constitutional unit derived from the compound represented by the formula (A-1), the acid generating agent (C), the acid diffusion controlling agent (E), the optional component (F) (the compound represented by the formula (A-1) or the resin comprising a constitutional unit derived from the compound represented by the formula (A-1)/the acid generating agent (C)/the acid diffusion controlling agent (E)/the optional component (F)) is preferably 50 to 99.4/0.001 to 49/0.001 to 49/0 to 49 in % by mass based on the solid content, more preferably 55 to 90/1 to 40/0.01 to 10/0 to 5, still more preferably 60 to 80/3 to 30/0.01 to 5/0 to 1, and particularly preferably 60 to 70/10 to 25/0.01 to 3/0.

The content ratio of each component is selected from each range so that the summation thereof is 100% by mass. By the above content ratio, performance such as sensitivity, resolution, and developability is even better.

The method for purifying the optical component forming composition of the present embodiment is not particularly limited, and, examples include a method involving dissolving each component in a solvent upon use into a homogenous solution, and then if required, filtering through a filter or the like with a pore diameter of about 0.2 μm, for example.

The optical component forming composition of the present embodiment can contain a resin within the range of not inhibiting the purpose of the present invention. Examples of the resin include, but not particularly limited to, a novolac resin, polyvinyl phenols, polyacrylic acid, polyvinyl alcohol, a styrene-maleic anhydride resin, an acrylic acid, vinyl alcohol or vinylphenol as a monomeric unit, or derivative thereof. The content of the resin is not particularly limited, and is arbitrarily adjusted according to the kind of the compound represented by the formula (A-1) or the resin comprising a constitutional unit derived from the compound represented by the formula (A-1) to be used, but is preferably 30 parts by mass or less per 100 parts by mass of the compound, more preferably 10 parts by mass or less, still more preferably 5 parts by mass or less, particularly preferably 0 parts by mass.

The cured product of the present embodiment is obtained by curing the above optical component forming composition and can be used as various resins. Such a cured product can be used for various purposes as a highly versatile material that confers various properties such as a high melting point, high refractive index and high transparency. The cured product can be obtained by using a publicly known method appropriate for each composition of the above composition, such as light irradiation or heating.

The cured product can be used as various synthetic resins such as an epoxy resin, a polycarbonate resin, and an acrylic resin and further as an optical component such as a lens or an optical sheet by exploiting functionality.

### EXAMPLES

The present embodiment will be more specifically described with reference to examples below. However, the present invention is not limited to these examples. Below, methods for measuring a compound and methods for evaluating optical component performance and the like in examples are presented.

### [Measurement Method]

### (1) Structure of Compound

The structure of the compound was verified by carrying out ¹H-NMR measurement under the following conditions using “Advance 600 II spectrometer” manufactured by Bruker.

Frequency: 400 MHz

Solvent: d6-DMSO (except for Synthesis Example 4)

Internal standard: TMS

Measurement temperature: 23° C.

### (2) Molecular Weight of Compound

The molecular weight of the compound was measured by GC-MS analysis using “Agilent 5975/6890N” manufactured by Agilent Technologies, Inc. or by LC-MS analysis using “Acquity UPLC/MALDI-Synapt HDMS” manufactured by Waters Corp.

### [Evaluation Method]

### (1) Organic Solvent Solubility Test of Compound

The solubility of the compound in propylene glycol monomethyl ether acetate was measured as the solubility of the compound in an organic solvent. The solubility was evaluated according to the following criteria utilizing the amount of dissolution in propylene glycol monomethyl ether acetate. The amount of dissolution was measured at 23° C. by precisely weighing the compound into a test tube, adding propylene glycol monomethyl ether acetate so as to attain a predetermined concentration, applying ultrasonic waves for 30 minutes in an ultrasonic cleaner, then visually observing the subsequent state of the fluid, and conducting evaluation on the basis of the concentration of the amount of complete dissolution.

A: 5.0% by mass Amount of dissolution

B: 3.0% by mass Amount of dissolution <5.0% by mass

C: Amount of dissolution <3.0% by mass

### (2) Storage Stability and Thin Film Formability of Optical Component Forming Composition

The storage stability of an optical component forming composition containing the compound was evaluated by leaving the resist composition to stand still for three days at 23° C. after preparation and then visually observing the optical component forming composition for the presence and absence of precipitates. The optical component forming composition after being left to stand still for three days was evaluated as “A” when it was a homogeneous solution without precipitates, and “C” when precipitates were observed.

A clean silicon wafer was spin coated with the optical component forming composition in a homogeneous state, and then prebaked (PB) in an oven of 110° C. to form an optical component forming film with a thickness of 1 μm. The prepared optical component forming composition was evaluated as “A” when the film formability was good, and “C” when the formed film had defects.

### (3) Evaluation of Refractive Index and Transparency

A clean silicon wafer was spin coated with the homogeneous optical component forming composition, and then PB in an oven of 110° C. to form a film with a thickness of 1 μm. The refractive index (X=589.3 nm) of the film at 25° C. was measured using a variable angle spectroscopic ellipsometer VASE manufactured by J. A. Woollam Co., Inc. The prepared film was evaluated as “A” when the refractive index was 1.8 or more, “B” when the refractive index was 1.6 or more and less than 1.8, and “C” when the refractive index was less than 1.6. Also, the film was evaluated as “A” when the transparency (X=632.8 nm) was 90% or more, and “C” when the transparency was less than 90%.

### SYNTHESIS EXAMPLES

### (Synthesis Example 1) Synthesis of Compound (BHPT)

In a glove box, to a 50 mL container, tellurium tetrachloride (5.39 g, 20 mmol) was fed, 10.8 g (100 mmol) of anisole was added, and the mixture was reacted at 160° C. for 6 hours under reflux conditions. The obtained product was dried under reduced pressure, and recrystallization was carried out twice using acetonitrile, followed by filtration to obtain orange crystals. The obtained crystals were dried under reduced pressure for 24 hours to obtain 5.95 g of BMPT (bis(4-methoxyphenyl)tellurium dichloride).

As a result of measuring the molecular weight of the obtained compound (BMPT) by the above measurement method (LC-MS), it was 414.

The following peaks were found by NMR measurement performed on the obtained compound (BMPT) under the above measurement conditions, and the compound was confirmed to have a chemical structure of the compound (BMPT) shown below.

δ (ppm) 7.0-7.9 (8H, Ph-H), 3.8 (6H, —CH₃)

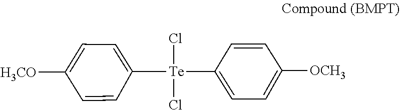

Then, to a container (internal capacity: 100 mL) equipped with a stirrer, a condenser tube, and a burette, 1.1 g (2.8 mmol) of bis(4-methoxyphenyl)tellurium dichloride and 18 ml of methylene dichloride were added, 3.9 g (15.75 mmol) of boron tribromide was dropped, and the mixture was reacted at −20° C. for 48 hours. The solution after reaction was dropped to a 0.5 N hydrochloric acid solution in an ice bath, and a yellow solid was recovered after filtration. The solid was dissolved in ethyl acetate, the solution was dehydrated by the addition of magnesium sulfate and then concentrated, and the residue was separated and purified by column chromatography to obtain 0.1 g of BHPT (bis(4-hydroxyphenyl)tellurium dichloride).

As a result of measuring the molecular weight of the obtained compound (BHPT) by the above measurement method (LC-MS), it was 386.

The following peaks were found by NMR measurement performed on the obtained compound (BHPT) under the above measurement conditions, and the compound was confirmed to have a chemical structure of the compound (BHPT) shown below.

δ (ppm) 10.2 (2H, —OH), 6.8-7.8 (8H, Ph-H)

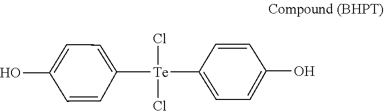

The solubility of the obtained compound (BHPT) in a safe solvent was evaluated by the above method. The results are shown in Table 1.

### (Synthesis Example 2) Synthesis of Compound (BHPT-ADBAC)

In a container (internal capacity: 200 mL) equipped with a stirrer, a condenser tube, and a burette, 3.9 g (10 mmol) of the compound (BHPT) obtained as mentioned above, 0.30 g (22 mmol) of potassium carbonate, and 0.64 g (2 mmol) of tetrabutyl ammonium bromide were dissolved in 50 ml of N-methylpyrrolidone, and the solution was stirred for 2 hours. After stirring, 6.3 g (22 mmol) of bromoacetic acid-2-methyladamantan-2-yl was further added thereto, and the mixture was reacted at 100° C. for 24 hours. After the reaction terminated, the reaction mixture was dropped to a 1 N aqueous hydrochloric acid solution, and the resulting black solid was filtered off and separated and purified by column chromatography to obtain 1.9 g of the following compound (BHPT-ADBAC: bis(4-(2-methyl-2-adamantyloxycarbonylmethoxy)phenyl)tellurium dichloride).

As a result of measuring the molecular weight of the obtained compound (BHPT-ADBAC) by the above measurement method (LC-MS), it was 798.

The following peaks were found by NMR measurement performed on the obtained compound (BHPT-ADBAC) under the above measurement conditions, and the compound was confirmed to have a chemical structure of the compound (BHPT-ADBAC) shown below.

δ (ppm) 6.8-8.1 (8H, Ph-H), 4.7-5.0 (4H, O—CH₂—C(═O)—), 1.2-2.7 (34H, C—H/Adamantane of methylene and methine)

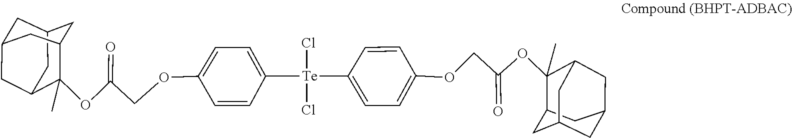

The solubility of the obtained compound in a safe solvent was evaluated by the above method. The results are shown in Table 1.

### (Synthesis Example 3) Synthesis of Compound (BHPT-BOC)

In a container (internal capacity: 200 mL) equipped with a stirrer, a condenser tube, and a burette, 3.9 g (10 mmol) of the compound (BHPT) obtained as mentioned above and 5.5 g (25 mmol) of di-t-butyl dicarbonate (manufactured by Sigma-Aldrich) were dissolved in 50 ml of N-methylpyrrolidone, 0.30 g (22 mmol) of potassium carbonate was added to the solution, and the mixture was reacted at 100° C. for 24 hours. After the reaction terminated, the reaction mixture was dropped to a 1 N aqueous hydrochloric acid solution, and the resulting black solid was filtered off and separated and purified by column chromatography to obtain 1.0 g of the following compound (BHPT-BOC: bis(tert-butoxycarboxyphenyl)tellurium dichloride).

As a result of measuring the molecular weight of the obtained compound (BHPT-BOC) by the above measurement method (LC-MS), it was 585.

The following peaks were found by NMR measurement performed on the obtained compound (BHPT-BOC) under the above measurement conditions, and the compound was confirmed to have a chemical structure of the compound (BHPT-BOC) shown below.

δ (ppm) 7.1-7.3 (8H, Ph-H), 1.4 (18H, C—CH₃)

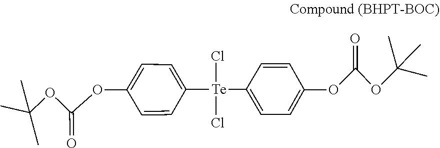

The solubility of the obtained compound in a safe solvent was evaluated by the above method. The results are shown in Table 1.

### (Synthesis Example 4) Synthesis of Compound (BHPT-EE)

In a container (internal capacity: 200 mL) equipped with a stirrer, a condenser tube, and a burette, 3.9 g (10 mmol) of the compound (BHPT) obtained as mentioned above and 1.8 g (25 mmol) of ethyl vinyl ether (manufactured by Tokyo Kasei Kogyo Co., Ltd.) were dissolved in 50 ml of N-methylpyrrolidone, 0.30 g (22 mmol) of potassium carbonate was added to the solution, and the mixture was reacted at 100° C. for 24 hours. After the reaction terminated, the reaction mixture was dropped to a 1 N aqueous hydrochloric acid solution, and the resulting black solid was filtered off and separated and purified by column chromatography to obtain 1.0 g of the following compound (BHPT-EE: bis(ethoxyethylphenyl)tellurium dichloride).

As a result of measuring the molecular weight of the obtained compound (BHPT-EE) by the above measurement method (LC-MS), it was 529.

The following peaks were found by NMR measurement performed on the obtained compound (BHPT-EE) under the above measurement conditions, and the compound was confirmed to have a chemical structure of the compound (BHPT-EE) shown below.

δ (ppm) 6.9-7.4 (8H, Ph-H), 5.6 (2H, CH), 1.6 (6H, —CH₃), 3.9 (4H, O—CH₂—), 1.2 (6H, —CH₃)

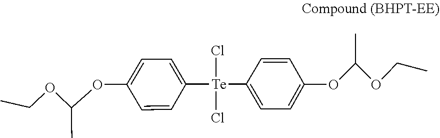

The solubility of the obtained compound in a safe solvent was evaluated by the above method. The results are shown in Table 1.

### (Synthesis Example 5) Synthesis of Compound (Ph-BHPT)

In a glove box, to a 50 mL container, tellurium tetrachloride (5.39 g, 20 mmol) was fed, 7.37 g (40 mmol) of 2-phenylanisole was added, and the mixture was reacted at 160° C. for 6 hours under reflux conditions. The obtained product was dried under reduced pressure, and recrystallization was carried out twice using acetonitrile, followed by filtration to obtain orange crystals. The obtained crystals were dried under reduced pressure for 24 hours to obtain 3.91 g of Ph-BMPT (bis(3-phenyl-4-methoxyphenyl)tellurium dichloride).

As a result of measuring the molecular weight of the obtained compound (Ph-BMPT) by the above measurement method (LC-MS), it was 465.

The following peaks were found by NMR measurement performed on the obtained compound (Ph-BMPT) under the above measurement conditions, and the compound was confirmed to have a chemical structure of the compound (Ph-BMPT) shown below.

δ (ppm) 7.0-8.1 (16H, Ph-H), 3.8 (6H, —CH₃)

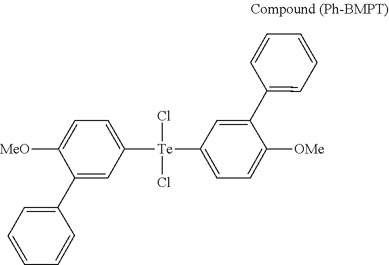

Then, to a container (internal capacity: 100 mL) equipped with a stirrer, a condenser tube, and a burette, 1.6 g (2.8 mmol) of Ph-BMPT and 25 ml of methylene dichloride were added, 3.9 g (15.75 mmol) of boron tribromide was dropped, and the mixture was reacted at −20° C. for 48 hours. The solution after reaction was dropped to a 0.5 N hydrochloric acid solution in an ice bath, and a yellow solid was recovered after filtration. The solid was dissolved in ethyl acetate, the solution was dehydrated by the addition of magnesium sulfate and then concentrated, and the residue was separated and purified by column chromatography to obtain 0.2 g of Ph-BHPT (bis(3-phenyl-4-hydroxyphenyl)tellurium dichloride).

As a result of measuring the molecular weight of the obtained compound (Ph-BHPT) by the above measurement method (LC-MS), it was 537.

The following peaks were found by NMR measurement performed on the obtained compound (Ph-BHPT) under the above measurement conditions, and the compound was confirmed to have a chemical structure of the compound (Ph-BHPT) shown below.

δ (ppm) 9.0 (2H, —OH), 7.0-7.5 (16H, Ph-H)

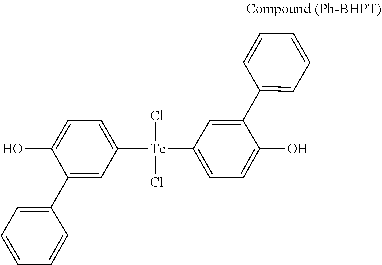

The solubility of the obtained compound in a safe solvent was evaluated by the above method. The results are shown in Table 1.

### (Synthesis Example 6) Synthesis of Compound (TDP)

In a glove box, to a 50 mL container, tellurium tetrachloride (6.74 g, 25 mmol) was fed, 3.29 g (35 mmol) of phenol was added, and the mixture was reacted at 160° C. for 6 hours under reflux conditions. The obtained product was dried under reduced pressure, and recrystallization was carried out twice using acetonitrile, followed by filtration to obtain brown crystals. The obtained crystals were dried under reduced pressure for 24 hours to obtain 3.60 g of TDP (4,4′-telluriumdiphenol).

As a result of measuring the molecular weight of the obtained compound (TDP) by the above measurement method (LC-MS), it was 314.

The following peaks were found by NMR measurement performed on the obtained compound (TDP) under the above measurement conditions, and the compound was confirmed to have a chemical structure of the compound (TDP) shown below.

δ (ppm) 6.8-7.7 (8H, Ph-H), 9.8 (2H, —OH)

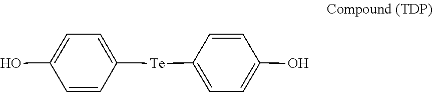

The solubility of the obtained compound in a safe solvent was evaluated by the above method. The results are shown in Table 1.

### (Synthesis Example 7) Synthesis of Compound (Ph-TDP)

In a glove box, to a 50 mL container, tellurium tetrachloride (6.74 g, 25 mmol) was fed, 6.96 g (35 mmol) of 2-phenol was added, and the mixture was reacted at 160° C. for 6 hours under reflux conditions. The obtained product was dried under reduced pressure, and recrystallization was carried out twice using acetonitrile, followed by filtration to obtain brown crystals. The obtained crystals were dried under reduced pressure for 24 hours to obtain 2.46 g of Ph-TDP (bis(3-phenyl-4-hydroxyphenyl)tellurium).

As a result of measuring the molecular weight of the obtained compound (Ph-TDP) by the above measurement method (LC-MS), it was 466.

The following peaks were found by NMR measurement performed on the obtained compound (Ph-TDP) under the above measurement conditions, and the compound was confirmed to have a chemical structure of the compound (Ph-TDP) shown below.

δ (ppm) 6.8-7.7 (16H, Ph-H), 9.8 (2H, —OH)

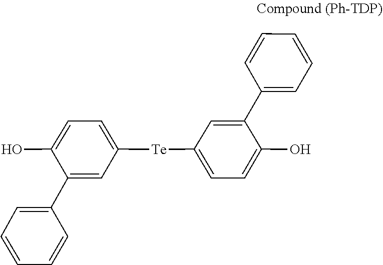

The solubility of the obtained compound in a safe solvent was evaluated by the above method. The results are shown in Table 1.

### (Synthesis Example 8) Synthesis of Compound (Ph-BHPT-ADBAC)

The same operations as in Synthesis Example 2 were performed except that 5.4 g (10 mmol) of the compound (Ph-BHPT) was used in place of 3.9 g (10 mmol) of the compound (BHPT), to obtain 1.28 g of a compound (Ph-BHPT-ADBAC) having a structure shown below.

As a result of measuring the molecular weight of the obtained compound (Ph-BHPT-ADBAC) by the above measurement method (LC-MS), it was 537.

The following peaks were found by NMR measurement performed on the obtained compound (Ph-BHPT-ADBAC) under the above measurement conditions, and the compound was confirmed to have a chemical structure of the compound (BHPT-ADBAC) shown below.

δ (ppm) 7.1-7.7 (16H, Ph-H), 5.0 (4H, O—CH₂—C(═O)—), 1.0-2.6 (34H, C—H/Adamantane of methylene and methine)

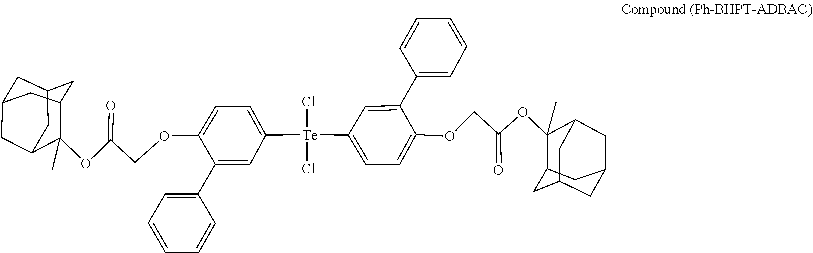

The solubility of the obtained compound in a safe solvent was evaluated by the above method. The results are shown in Table 1.

### (Synthesis Example 9) Synthesis of Compound (TDP-ADBAC)

The same operations as in Synthesis Example 2 were performed except that 3.2 g (10 mmol) of the compound (TDP) was used in place of 3.9 g (10 mmol) of the compound (BHPT), to obtain 1.46 g of a compound (TDP-ADBAC) having a structure shown below.

As a result of measuring the molecular weight of the obtained compound (TDP-ADBAC) by the above measurement method (LC-MS), it was 726.

The following peaks were found by NMR measurement performed on the obtained compound (TDP-ADBAC) under the above measurement conditions, and the compound was confirmed to have a chemical structure of the compound (TDP-ADBAC) shown below.

δ (ppm) 7.0-7.4 (8H, Ph-H), 5.0 (4H, O—CH₂—C(═O)—), 1.0-2.6 (34H, C—H/Adamantane of methylene and methine)

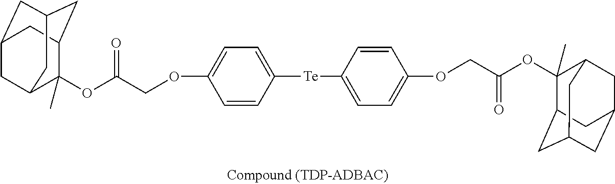

The solubility of the obtained compound in a safe solvent was evaluated by the above method. The results are shown in Table 1.

### (Synthesis Example 10) Synthesis of Compound (Ph-TDP-ADBAC)

The same operations as in Synthesis Example 2 were performed except that 4.7 g (10 mmol) of the compound (Ph-TDP) was used in place of 3.9 g (10 mmol) of the compound (BHPT), to obtain 1.70 g of a compound (Ph-TDP-ADBAC) having a structure shown below.

As a result of measuring the molecular weight of the obtained compound (Ph-TDP-ADBAC) by the above measurement method (LC-MS), it was 879.

The following peaks were found by NMR measurement performed on the obtained compound (Ph-TDP-ADBAC) under the above measurement conditions, and the compound was confirmed to have a chemical structure of the compound (Ph-TDP-ADBAC) shown below.

δ (ppm) 7.1-7.7 (16H, Ph-H), 5.0 (4H, O—CH₂—C(═O)—), 1.0-2.6 (34H, C—H/Adamantane of methylene and methine)

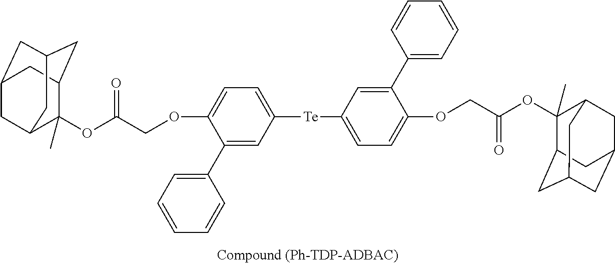

The solubility of the obtained compound in a safe solvent was evaluated by the above method. The results are shown in Table 1.

### (Synthesis Example 11) Synthesis of Compound (Ph-TDP-BOC)

The same operations as in Synthesis Example 3 were performed except that 4.7 g (10 mmol) of the compound (Ph-TDP) was used in place of 3.9 g (10 mmol) of the compound (BHPT), to obtain 1.14 g of a compound (Ph-TDP-BOC) having a structure shown below.

As a result of measuring the molecular weight of the obtained compound (Ph-TDP-BOC) by the above measurement method (LC-MS), it was 666.

The following peaks were found by NMR measurement performed on the obtained compound (Ph-TDP-BOC) under the above measurement conditions, and the compound was confirmed to have a chemical structure of the compound (Ph-TDP-BOC) shown below.

δ (ppm) 7.3-7.7 (8H, Ph-H), 1.4 (18H, C—CH₃)

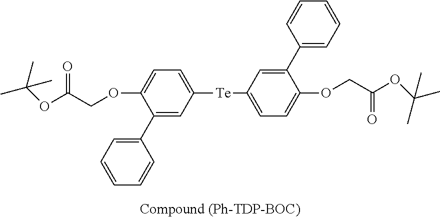

The solubility of the obtained compound in a safe solvent was evaluated by the above method. The results are shown in Table 1.

### (Synthesis Example 12) Synthesis of Compound (Ph-TDP-EE)

The same operations as in Synthesis Example 4 were performed except that 4.7 g (10 mmol) of the compound (Ph-TDP) was used in place of 3.9 g (10 mmol) of the compound (BHPT), to obtain 1.16 g of a compound (Ph-TDP-EE) having a structure shown below.

As a result of measuring the molecular weight of the obtained compound (Ph-TDP-EE) by the above measurement method (LC-MS), it was 610.

The following peaks were found by NMR measurement performed on the obtained compound (Ph-TDP-EE) under the above measurement conditions, and the compound was confirmed to have a chemical structure of the compound (Ph-TDP-EE) shown below.

δ (ppm) 7.1-7.7 (16H, Ph-H), 5.6 (2H, CH), 1.6 (6H, —CH₃), 3.9 (4H, O—CH₂—), 1.2 (6H, —CH₃)

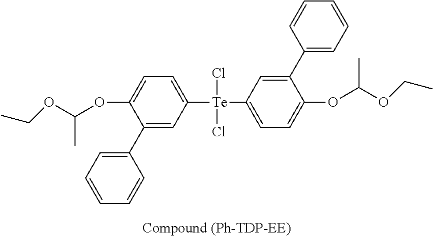

The solubility of the obtained compound in a safe solvent was evaluated by the above method. The results are shown in Table 1.

### (Synthesis Example 13) Synthesis of R1-BHPT

To a container (internal capacity: 100 ml) equipped with a stirrer, a condenser tube, and a burette, 8.1 g (21 mmol) of the compound (BHPT), 0.7 g (42 mmol) of paraformaldehyde, 50 ml of glacial acetic acid, and 50 ml of PGME were fed, 8 ml of 95% sulfuric acid was added, and the reaction solution was stirred at 100° C. for 6 hours and reacted. Next, the reaction solution was concentrated. The reaction product was precipitated by the addition of 1000 ml of methanol. After cooling to room temperature, the precipitates were separated by filtration. The obtained solid matter was subjected to filtration, dried, and then separated and purified by column chromatography to obtain 5.6 g of the objective resin (R1-BHPT) having a structure represented by the following formula.

As a result of measuring the molecular weight in terms of polystyrene of the obtained resin (R1-BHPT) by the above method, it was Mn: 587, Mw: 1216, Mw/Mn: 2.07.

The following peaks were found by NMR measurement performed on the obtained resin(R1-BHPT) under the above measurement conditions, and the resin was confirmed to have a chemical structure of the following formula (R1-BHPT).

δ (ppm) 10.2 (2H, —OH), 6.8-7.8 (8H, Ph-H), 4.1 (2H, —CH₂)

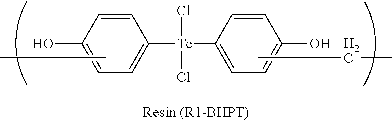

The solubility of the obtained compound in a safe solvent was evaluated by the above method. The results are shown in Table 1.

### (Synthesis Example 14) Synthesis of R2-BHPT

The same operations as in Synthesis Example 13 were performed except that 7.6 g (42 mmol) of 4-biphenylcarboxyaldehyde (manufactured by Mitsubishi Gas Chemical Company Inc.) was used in place of 0.7 g (42 mmol) of paraformaldehyde, to obtain 5.7 g of the objective resin (R2-BHPT) having a structure represented by the following formula.

As a result of measuring the molecular weight in terms of polystyrene of the obtained resin (R2-BHPT) by the above method, it was Mn: 405, Mw: 880, Mw/Mn: 2.17.

The following peaks were found by NMR measurement performed on the obtained resin (R2-BHPT) under the above measurement conditions, and the resin was confirmed to have a chemical structure of the following formula (R2-BHPT).

δ (ppm) 10.2 (2H, —OH), 6.8-7.8 (17H, Ph-H), 4.5 (1H, —CH)

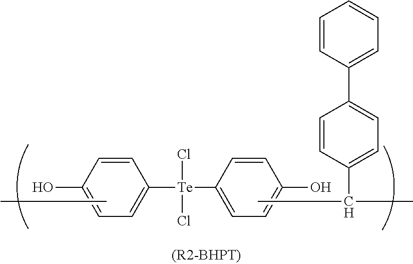

The solubility of the obtained compound in a safe solvent was evaluated by the above method. The results are shown in Table 1.

### (Synthesis Example 15) Synthesis of R1-BHPT-ADBAC

The same operations as in Synthesis Example 13 were performed except that 16.8 g of the compound (BHPT-ADBAC) was used in place of 8.1 g (21 mmol) of the compound (BHPT), to obtain 5.0 g of the objective resin (R1-BHPT-ADBAC) having a structure represented by the following formula.

As a result of measuring the molecular weight in terms of polystyrene of the obtained resin (R1-BHPT-ADBAC) by the above method, it was Mn: 1045, Mw: 2330, Mw/Mn: 2.23.

The following peaks were found by NMR measurement performed on the obtained resin (R1-BHPT-ADBAC) under the above measurement conditions, and the resin was confirmed to have a chemical structure of the following formula (R1-BHPT-ADBAC).

δ (ppm) 6.8-8.1 (8H, Ph-H), 4.7-5.0 (4H, O—CH₂—C(═O)—), 1.2-2.7 (34H, C—H/Adamantane of methylene and methine), 4.1 (2H, —CH₂)

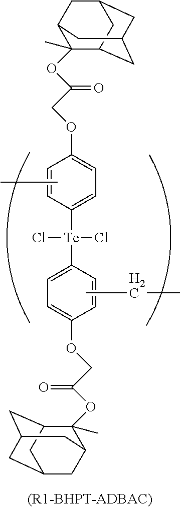

The solubility of the obtained compound in a safe solvent was evaluated by the above method. The results are shown in Table 1.

### (Synthesis Example 16) Synthesis of R2-BHPT-ADBAC

The same operations as in Synthesis Example 15 were performed except that 7.6 g (42 mmol) of 4-biphenylcarboxyaldehyde (manufactured by Mitsubishi Gas Chemical Company Inc.) was used in place of 0.7 g (42 mmol) of paraformaldehyde, to obtain 10.4 g of the objective resin (R2-BHPT-ADBAC) having a structure represented by the following formula.

As a result of measuring the molecular weight in terms of polystyrene of the obtained resin (R2-BHPT-ADBAC) by the above method, it was Mn: 840, Mw: 1819, Mw/Mn: 2.16.

The following peaks were found by NMR measurement performed on the obtained resin (R2-BHPT-ADBAC) under the above measurement conditions, and the resin was confirmed to have a chemical structure of the following formula (R2-BHPT-ADBAC).

δ (ppm) 6.8-8.1 (17H, Ph-H), 4.7-5.0 (4H, O—CH₂—C(═O)—), 1.2-2.7 (34H, C—H/Adamantane of methylene and methine), 4.5 (1H, —CH)

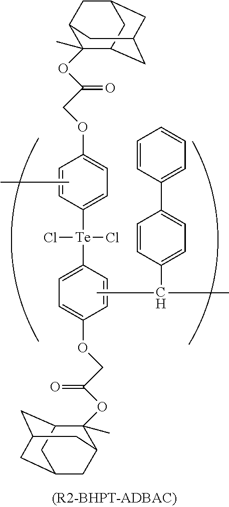

The solubility of the obtained compound in a safe solvent was evaluated by the above method. The results are shown in Table 1.

### (Synthesis Example 17) Synthesis of R1-BHPT-BOC

The same operations as in Synthesis Example 13 were performed except that 12.3 g of the compound (BHPT-BOC) was used in place of 8.1 g (21 mmol) of the compound (BHPT), to obtain 7.6 g of the objective resin (R1-BHPT-BOC) having a structure represented by the following formula.

As a result of measuring the molecular weight in terms of polystyrene of the obtained resin (R1-BHPT-BOC) by the above method, it was Mn: 768, Mw: 1846, Mw/Mn: 2.40.

The following peaks were found by NMR measurement performed on the obtained resin (R1-BHPT-BOC) under the above measurement conditions, and the resin was confirmed to have a chemical structure of the following formula (R1-BHPT-BOC).

δ (ppm) 7.1-7.3 (8H, Ph-H), 1.4 (18H, C—CH₃), 4.1 (2H, —CH₂)

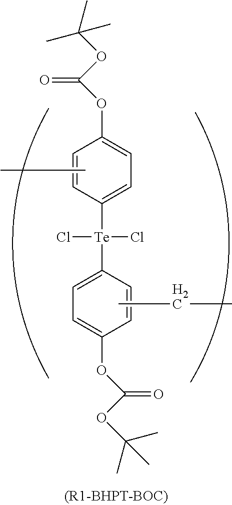

The solubility of the obtained compound in a safe solvent was evaluated by the above method. The results are shown in Table 1.

### (Synthesis Example 18) Synthesis of R2-BHPT-BOC

The same operations as in Synthesis Example 17 were performed except that 7.6 g (42 mmol) of 4-biphenylcarboxyaldehyde (manufactured by Mitsubishi Gas Chemical Company Inc.) was used in place of 0.7 g (42 mmol) of paraformaldehyde, to obtain 3.7 g of the objective resin (R2-BHPT-BOC) having a structure represented by the following formula.

As a result of measuring the molecular weight in terms of polystyrene of the obtained resin (R2-BHPT-BOC) by the above method, it was Mn: 620, Mw: 1336, Mw/Mn: 2.15.

The following peaks were found by NMR measurement performed on the obtained resin (R2-BHPT-BOC) under the above measurement conditions, and the resin was confirmed to have a chemical structure of the following formula (R2-BHPT-BOC).

δ (ppm) 7.1-7.3 (17H, Ph-H), 1.4 (18H, C—CH₃), 4.5 (1H, —CH)

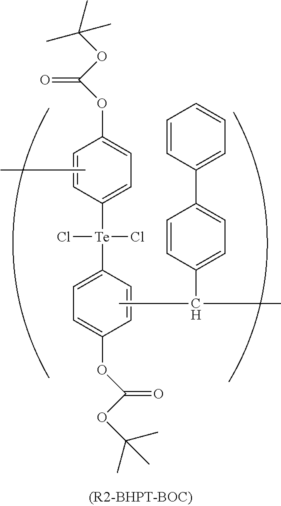

The solubility of the obtained compound in a safe solvent was evaluated by the above method. The results are shown in Table 1.

### (Synthesis Example 19) Synthesis of R1-BHPT-EE

The same operations as in Synthesis Example 13 were performed except that 11.1 g of the compound (BHPT-EE) was used in place of 8.1 g (21 mmol) of the compound (BHPT), to obtain 7.8 g of the objective resin (R1-BHPT-EE) having a structure represented by the following formula.

As a result of measuring the molecular weight in terms of polystyrene of the obtained resin (R1-BHPT-EE) by the above method, it was Mn: 694, Mw: 1548, Mw/Mn: 2.23.

The following peaks were found by NMR measurement performed on the obtained resin (R1-BHPT-EE) under the above measurement conditions, and the resin was confirmed to have a chemical structure of the following formula (R1-BHPT-EE).

δ (ppm) 6.9-7.4 (8H, Ph-H), 5.6 (2H, CH), 1.6 (6H, —CH₃), 3.9 (4H, O—CH₂—), 1.2 (6H, —CH₃), 4.1 (2H, —CH₂)

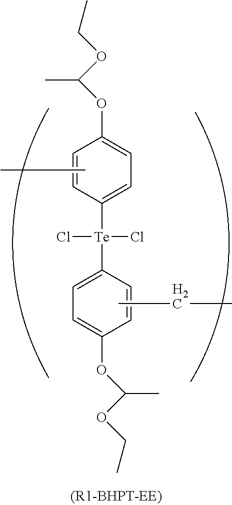

The solubility of the obtained compound in a safe solvent was evaluated by the above method. The results are shown in Table 1.

### (Synthesis Example 20) Synthesis of R2-BHPT-EE

The same operations as in Synthesis Example 19 were performed except that 7.6 g (42 mmol) of 4-biphenylcarboxyaldehyde (manufactured by Mitsubishi Gas Chemical Company Inc.) was used in place of 0.7 g (42 mmol) of paraformaldehyde, to obtain 3.6 g of the objective resin (R2-BHPT-EE) having a structure represented by the following formula.

As a result of measuring the molecular weight in terms of polystyrene of the obtained resin (R2-BHPT-EE) by the above method, it was Mn: 610, Mw: 1208, Mw/Mn: 1.98.

The following peaks were found by NMR measurement performed on the obtained resin (R2-BHPT-EE) under the above measurement conditions, and the resin was confirmed to have a chemical structure of the following formula (R2-BHPT-EE).

δ (ppm) 6.9-7.4 (17H, Ph-H), 5.6 (2H, CH), 1.6 (6H, —CH₃), 3.9 (4H, O—CH₂—), 1.2 (6H, —CH₃), 4.5 (1H, —CH)

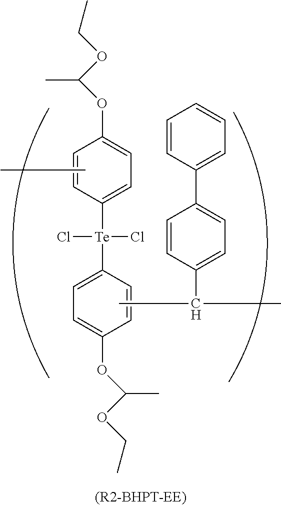

The solubility of the obtained compound in a safe solvent was evaluated by the above method. The results are shown in Table 1.

### (Synthesis Example 21) Synthesis of R1-Ph-BHPT

The same operations as in Synthesis Example 13 were performed except that 11.3 g of the compound (Ph-BHPT) was used in place of 8.1 g (21 mmol) of the compound (BHPT), to obtain 7.0 g of the objective resin (R1-Ph-BHPT) having a structure represented by the following formula.

As a result of measuring the molecular weight in terms of polystyrene of the obtained resin (R1-Ph-BHPT) by the above method, it was Mn: 764, Mw: 1695, Mw/Mn: 2.22.

The following peaks were found by NMR measurement performed on the obtained resin (R1-Ph-BHPT) under the above measurement conditions, and the resin was confirmed to have a chemical structure of the following formula (R1-Ph-BHPT).

δ (ppm) 9.0 (2H, —OH), 7.0-7.5 (16H, Ph-H), 4.1 (2H, —CH₂)

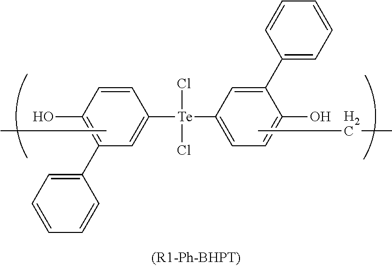

The solubility of the obtained compound in a safe solvent was evaluated by the above method. The results are shown in Table 1.

### (Synthesis Example 22) Synthesis of R2-Ph-BHPT

The same operations as in Synthesis Example 21 were performed except that 7.6 g (42 mmol) of 4-biphenylcarboxyaldehyde (manufactured by Mitsubishi Gas Chemical Company Inc.) was used in place of 0.7 g (42 mmol) of paraformaldehyde, to obtain 3.4 g of the objective resin (R2-Ph-BHPT) having a structure represented by the following formula.

As a result of measuring the molecular weight in terms of polystyrene of the obtained resin (R2-Ph-BHPT) by the above method, it was Mn: 672, Mw: 1345, Mw/Mn: 2.00.

The following peaks were found by NMR measurement performed on the obtained resin (R2-Ph-BHPT) under the above measurement conditions, and the resin was confirmed to have a chemical structure of the following formula (R2-Ph-BHPT).

δ (ppm) 9.0 (2H, —OH), 7.0-7.5 (25H, Ph-H), 4.5 (1H, —CH)

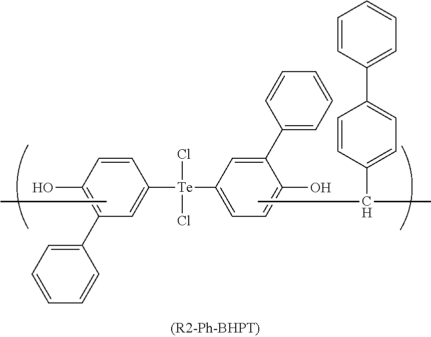

The solubility of the obtained compound in a safe solvent was evaluated by the above method. The results are shown in Table 1.

### (Synthesis Example 23) Synthesis of R1-TDP

The same operations as in Synthesis Example 13 were performed except that 6.6 g of the compound (TDP) was used in place of 8.1 g (21 mmol) of the compound (BHPT), to obtain 4.6 g of the objective resin (R1-TDP) having a structure represented by the following formula.

As a result of measuring the molecular weight in terms of polystyrene of the obtained resin (R1-TDP) by the above method, it was Mn: 449, Mw: 995, Mw/Mn: 2.22.

The following peaks were found by NMR measurement performed on the obtained resin (R1-TDP) under the above measurement conditions, and the resin was confirmed to have a chemical structure of the following formula (R1-TDP).

δ (ppm) 6.8-7.7 (8H, Ph-H), 9.8 (2H, —OH), 4.1 (2H, —CH₂)

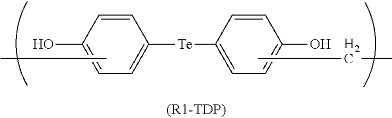

The solubility of the obtained compound in a safe solvent was evaluated by the above method. The results are shown in Table 1.

### (Synthesis Example 24) Synthesis of R2-TDP

The same operations as in Synthesis Example 23 were performed except that 7.6 g (42 mmol) of 4-biphenylcarboxyaldehyde (manufactured by Mitsubishi Gas Chemical Company Inc.) was used in place of 0.7 g (42 mmol) of paraformaldehyde, to obtain 2.0 g of the objective resin (R2-TDP) having a structure represented by the following formula.

As a result of measuring the molecular weight in terms of polystyrene of the obtained resin (R2-TDP) by the above method, it was Mn: 414, Mw: 922, Mw/Mn: 2.23.

The following peaks were found by NMR measurement performed on the obtained resin (R2-TDP) under the above measurement conditions, and the resin was confirmed to have a chemical structure of the following formula (R2-TDP).

δ (ppm) 6.8-7.7 (17H, Ph-H), 9.8 (2H, —OH), 4.5 (1H, —CH)

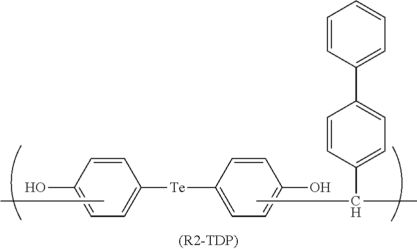

The solubility of the obtained compound in a safe solvent was evaluated by the above method. The results are shown in Table 1.

### (Synthesis Example 25) Synthesis of R1-Ph-TDP

The same operations as in Synthesis Example 13 were performed except that 9.8 g of the compound (Ph-TDP) was used in place of 8.1 g (21 mmol) of the compound (BHPT), to obtain 6.9 g of the objective resin (R1-Ph-TDP) having a structure represented by the following formula.

As a result of measuring the molecular weight in terms of polystyrene of the obtained resin (R1-Ph-TDP) by the above method, it was Mn: 665, Mw: 1474, Mw/Mn: 2.22.

The following peaks were found by NMR measurement performed on the obtained resin (R1-Ph-TDP) under the above measurement conditions, and the resin was confirmed to have a chemical structure of the following formula (R1-Ph-TDP).

δ (ppm) 6.8-7.7 (16H, Ph-H), 9.8 (2H, —OH), 4.1 (2H, —CH₂)

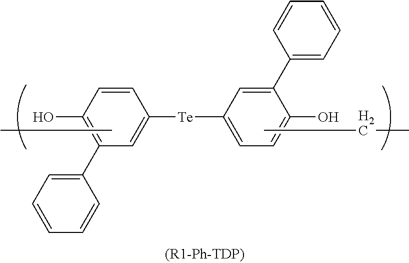

The solubility of the obtained compound in a safe solvent was evaluated by the above method. The results are shown in Table 1.

### (Synthesis Example 26) Synthesis of R2-Ph-TDP

The same operations as in Synthesis Example 21 were performed except that 7.6 g (42 mmol) of 4-biphenylcarboxyaldehyde (manufactured by Mitsubishi Gas Chemical Company Inc.) was used in place of 0.7 g (42 mmol) of paraformaldehyde, to obtain 3.2 g of the objective resin (R2-Ph-TDP) having a structure represented by the following formula.

As a result of measuring the molecular weight in terms of polystyrene of the obtained resin (R2-Ph-TDP) by the above method, it was Mn: 608, Mw: 1395, Mw/Mn: 2.29.

The following peaks were found by NMR measurement performed on the obtained resin (R2-Ph-TDP) under the above measurement conditions, and the resin was confirmed to have a chemical structure of the following formula (R2-Ph-TDP).

δ (ppm) 6.8-7.7 (25H, Ph-H), 9.8 (2H, —OH), 4.5 (1H, —CH)

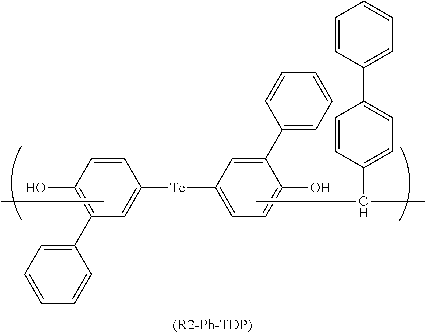

The solubility of the obtained compound in a safe solvent was evaluated by the above method. The results are shown in Table 1.

### (Synthesis Example 27) Synthesis of R1-Ph-BHPT-ADBAC

The same operations as in Synthesis Example 13 were performed except that 20.0 g of the compound (Ph-BHPT-ADBAC) was used in place of 8.1 g (21 mmol) of the compound (BHPT), to obtain 5.0 g of the objective resin (R1-Ph-BHPT-ADBAC) having a structure represented by the following formula.

As a result of measuring the molecular weight in terms of polystyrene of the obtained resin (R1-Ph-BHPT-ADBAC) by the above method, it was Mn: 1045, Mw: 2330, Mw/Mn: 2.23.

The following peaks were found by NMR measurement performed on the obtained resin (R1-Ph-BHPT-ADBAC) under the above measurement conditions, and the resin was confirmed to have a chemical structure of the following formula (R1-Ph-BHPT-ADBAC).

δ (ppm) 6.8-8.1 (8H, Ph-H), 4.7-5.0 (4H, O—CH₂—C(═O)—), 1.2-2.7 (34H, C—H/Adamantane of methylene and methine), 4.1 (2H, —CH₂)

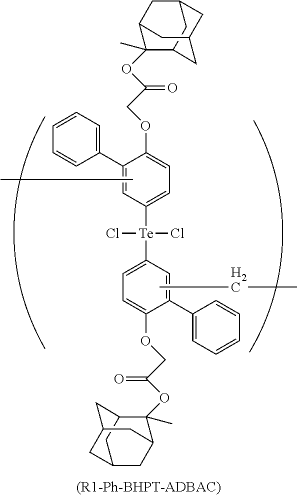

The solubility of the obtained compound in a safe solvent was evaluated by the above method. The results are shown in Table 1.

### (Synthesis Example 28) Synthesis of R2-Ph-BHPT-ADBAC

The same operations as in Synthesis Example 27 were performed except that 7.6 g (42 mmol) of 4-biphenylcarboxyaldehyde (manufactured by Mitsubishi Gas Chemical Company Inc.) was used in place of 0.7 g (42 mmol) of paraformaldehyde, to obtain 6.0 g of the objective resin (R2-Ph-BHPT-ADBAC) having a structure represented by the following formula.

As a result of measuring the molecular weight in terms of polystyrene of the obtained resin (R2-Ph-BHPT-ADBAC) by the above method, it was Mn: 1188, Mw: 2394, Mw/Mn: 2.02.

The following peaks were found by NMR measurement performed on the obtained resin (R2-Ph-BHPT-ADBAC) under the above measurement conditions, and the resin was confirmed to have a chemical structure of the following formula (R2-Ph-BHPT-ADBAC).

δ (ppm) 7.1-7.7 (25H, Ph-H), 5.0 (4H, O—CH2-C(═O)—), 1.0-2.6 (34H, C—H/Adamantane of methylene and methine), 4.5 (1H, —CH)

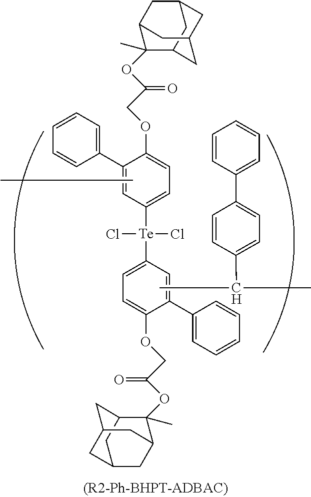

The solubility of the obtained compound in a safe solvent was evaluated by the above method. The results are shown in Table 1.

### (Synthesis Example 29) Synthesis of R1-TDP-ADBAC

The same operations as in Synthesis Example 13 were performed except that 15.3 g of the compound (TDP-ADBAC) was used in place of 8.1 g (21 mmol) of the compound (BHPT), to obtain 11.4 g of the objective resin (R1-TDP-ADBAC) having a structure represented by the following formula.

As a result of measuring the molecular weight in terms of polystyrene of the obtained resin (R1-TDP-ADBAC) by the above method, it was Mn: 954, Mw: 2148, Mw/Mn: 2.25.

The following peaks were found by NMR measurement performed on the obtained resin (R1-TDP-ADBAC) under the above measurement conditions, and the resin was confirmed to have a chemical structure of the following formula (R1-TDP-ADBAC).

δ (ppm) 7.0-7.4 (8H, Ph-H), 5.0 (4H, O—CH2-C(═O)—), 1.0-2.6 (34H, C—H/Adamantane of methylene and methine), 4.1 (2H, —CH2)

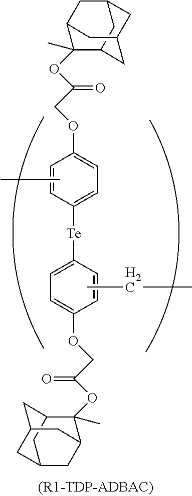

The solubility of the obtained compound in a safe solvent was evaluated by the above method. The results are shown in Table 1.

### (Synthesis Example 30) Synthesis of R2-TDP-ADBAC

The same operations as in Synthesis Example 29 were performed except that 7.6 g (42 mmol) of 4-biphenylcarboxyaldehyde (manufactured by Mitsubishi Gas Chemical Company Inc.) was used in place of 0.7 g (42 mmol) of paraformaldehyde, to obtain 4.6 g of the objective resin (R2-TDP-ADBAC) having a structure represented by the following formula.

As a result of measuring the molecular weight in terms of polystyrene of the obtained resin (R2-TDP-ADBAC) by the above method, it was Mn: 910, Mw: 1805, Mw/Mn: 1.98.

The following peaks were found by NMR measurement performed on the obtained resin (R2-TDP-ADBAC) under the above measurement conditions, and the resin was confirmed to have a chemical structure of the following formula (R2-TDP-ADBAC).

δ (ppm) 7.0-7.4 (17H, Ph-H), 5.0 (4H, O—CH2-C(═O)—), 1.0-2.6 (34H, C—H/Adamantane of methylene and methine), 4.5 (1H, —CH)

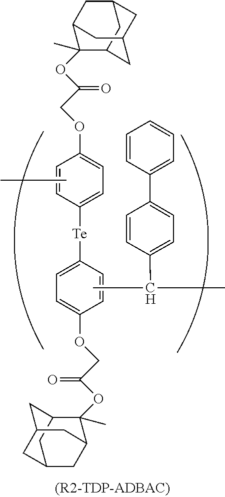

The solubility of the obtained compound in a safe solvent was evaluated by the above method. The results are shown in Table 1.

### (Synthesis Example 31) Synthesis of R1-Ph-TDP-ADBAC

The same operations as in Synthesis Example 13 were performed except that 18.5 g of the compound (Ph-TDP-ADBAC) was used in place of 8.1 g (21 mmol) of the compound (BHPT), to obtain 12.0 g of the objective resin (R1-Ph-TDP-ADBAC) having a structure represented by the following formula.

As a result of measuring the molecular weight in terms of polystyrene of the obtained resin (R1-Ph-TDP-ADBAC) by the above method, it was Mn: 1152, Mw: 2570, Mw/Mn: 2.23.

The following peaks were found by NMR measurement performed on the obtained resin (R1-Ph-TDP-ADBAC) under the above measurement conditions, and the resin was confirmed to have a chemical structure of the following formula (R1-Ph-TDP-ADBAC).

δ (ppm) 7.1-7.7 (16H, Ph-H), 5.0 (4H, O—CH₂—C(═O)—), 1.0-2.6 (34H, C—H/Adamantane of methylene and methine), 4.1 (2H, —CH2)

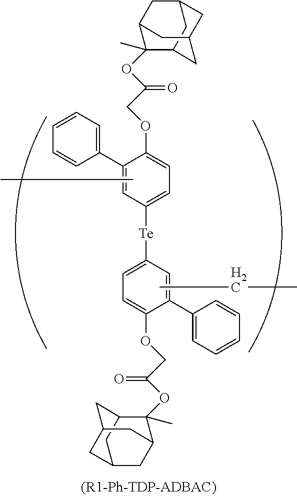

The solubility of the obtained compound in a safe solvent was evaluated by the above method. The results are shown in Table 1.

### (Synthesis Example 32) Synthesis of R2-Ph-TDP-ADBAC

The same operations as in Synthesis Example 31 were performed except that 7.6 g (42 mmol) of 4-biphenylcarboxyaldehyde (manufactured by Mitsubishi Gas Chemical Company Inc.) was used in place of 0.7 g (42 mmol) of paraformaldehyde, to obtain 5.6 g of the objective resin (R2-Ph-TDP-ADBAC) having a structure represented by the following formula.

As a result of measuring the molecular weight in terms of polystyrene of the obtained resin (R2-Ph-TDP-ADBAC) by the above method, it was Mn: 1100, Mw: 2205, Mw/Mn: 2.004.

The following peaks were found by NMR measurement performed on the obtained resin (R2-Ph-TDP-ADBAC) under the above measurement conditions, and the resin was confirmed to have a chemical structure of the following formula (R2-Ph-TDP-ADBAC).

δ (ppm) 7.1-7.7 (25H, Ph-H), 5.0 (4H, O—CH₂—C(═O)—), 1.0-2.6 (34H, C—H/Adamantane of methylene and methine), 4.5 (1H, —CH)

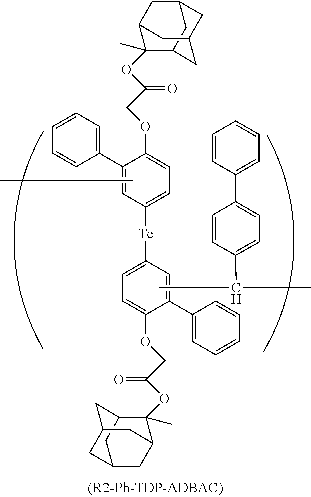

The solubility of the obtained compound in a safe solvent was evaluated by the above method. The results are shown in Table 1.

### (Synthesis Example 33) Synthesis of Resin (BHPT-co-ADTBA)

In a 100 mL container, 0.58 g (1.5 mmol) of the compound (BHPT) was placed, 0.05 g (0.15 mmol) of tetrabutyl ammonium bromide, 0.28 g (2 mmol) of potassium carbonate, and 2 ml of N-methylpyrrolidone were added, and the mixture was stirred at 80° C. for 2 hours. Next, 0.547 g (1.0 mmol) of ADTBA (1,3,5-adamantane tribromoacetate) was dissolved in 1 ml of N-methylpyrrolidone, and the solution was reacted at 80° C. for 48 hours. The obtained reaction product was dropped to 1 N HCl to obtain brown crystals. The crystals were filtered and then dried under reduced pressure to obtain 0.40 g of the objective resin (BHPT-co-ADTBA).

As a result of measuring the molecular weight in terms of polystyrene of the obtained resin (BHPT-co-ADTBA) by the above method, it was Mn: 750, Mw: 1350, Mw/Mn: 1.80.

The following peaks were found by NMR measurement performed on the obtained resin (BHPT-co-ADTBA) under the above measurement conditions, and the resin was confirmed to have a chemical structure of the following formula (BHPT-co-ADTBA).

δ (ppm) 6.9-7.4 (4H, Ph-H), 4.6 (4H, —O—CH₂—CO—), 4.3 (2H, —CH₂—Br), 1.2-3.4 (13H, C—H/Adamantane of methylene and methine)

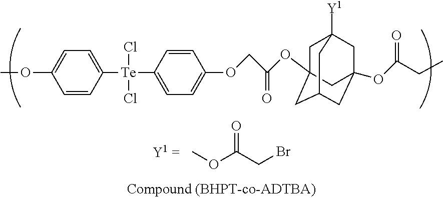

The solubility of the obtained compound in a safe solvent was evaluated by the above method. The results are shown in Table 1.

### (Synthesis Example 34) Synthesis of Resin (TDP-co-ADTBA)

The same operations as in Synthesis Example 33 were performed except that 0.47 g of the compound (TDP) was used in place of 0.58 g (1.5 mmol) of the compound (BHPT), to obtain 0.36 g of the objective resin (TDP-co-ADTBA) having a structure represented by the following formula.

As a result of measuring the molecular weight in terms of polystyrene of the obtained resin (TDP-co-ADTBA) by the above method, it was Mn: 680, Mw: 1238, Mw/Mn: 1.82.

The following peaks were found by NMR measurement performed on the obtained resin (TDP-co-ADTBA) under the above measurement conditions, and the resin was confirmed to have a chemical structure of the following formula (TDP-co-ADTBA).

δ (ppm) 6.9-7.4 (4H, Ph-H), 4.6 (4H, —O—CH₂—CO—), 4.3 (2H, —CH₂—Br), 1.2-3.4 (13H, C—H/Adamantane of methylene and methine)

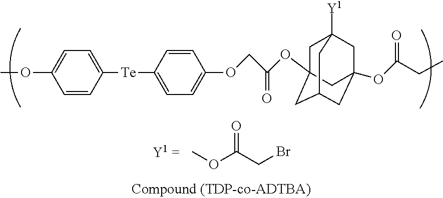

The solubility of the obtained compound in a safe solvent was evaluated by the above method. The results are shown in Table 1.

### (Synthesis Example 35) Synthesis of Resin (DMB-co-TeCl2-OH)

In a glove box, to a 100 ml container, 5.39 g (20 mmol) of tellurium tetrachloride was fed, 2.8 g (20 mmol) of 1,3-dimethoxybenzene, 5.9 g (44 mmol) of aluminum trichloride, and 20 ml of chloroform were added, and the mixture was reacted for 24 hours under ice cooling. The obtained product was dried under reduced pressure, and recrystallization was carried out twice using acetonitrile, followed by filtration. The obtained crystals were dried under reduced pressure for 24 hours to obtain 3.0 g of a resin (DMB-co-TeCl2).

As a result of measuring the molecular weight in terms of polystyrene of the obtained resin (DMB-co-TeCl2) by the above method, it was Mn: 39820, Mw: 62910, Mw/Mn: 1.58.

The following peaks were found by NMR measurement performed on the obtained resin (DMB-co-TeCl2) under the above measurement conditions, and the resin was confirmed to have a chemical structure of the following formula (DMB-co-TeCl2).

δ (ppm) 6.0-7.2 (2H, Ph-H), 3.6 (6H, —CH₃)

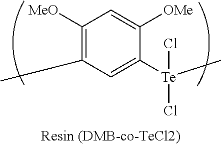

As a result of evaluating the solubility of the obtained compound in PGMEA by the above measurement method, it was 5% by mass or more (evaluation A). Thus, the compound was evaluated as having excellent solubility.

Then, to a container (internal capacity: 100 mL) equipped with a stirrer, a condenser tube, and a burette, 0.78 g of the resin (DMB-co-TeCl2) and 15 ml of chloroform were added, 3.9 g (15.75 mmol) of boron tribromide was dropped, and the mixture was reacted at −20° C. for 48 hours. The solution after reaction was dropped to a 1.0 N hydrochloric acid solution in an ice bath, and a black solid was recovered after filtration. The solid was dissolved in ethyl acetate, the solution was dehydrated by the addition of magnesium sulfate and then concentrated, and the residue was separated and purified by column chromatography to obtain 0.4 g of a resin (DMB-co-TeCl2-OH).

As a result of measuring the molecular weight in terms of polystyrene of the obtained resin (DMB-co-TeCl2-OH) by the above method, it was Mn: 39800, Mw: 62880, Mw/Mn: 1.58.

The following peaks were found by NMR measurement performed on the obtained resin (DMB-co-TeCl2-OH) under the above measurement conditions, and the resin was confirmed to have a chemical structure of the resin (DMB-co-TeCl2-OH) shown below.

δ (ppm) 9.0 (2H, —OH), 6.4-7.0 (2H, Ph-H)

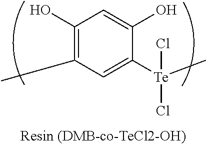

The solubility of the obtained compound in a safe solvent was evaluated by the above method. The results are shown in Table 1.

### (Synthesis Example 36) Synthesis of Resin (Re-co-Te)

In a glove box, to a 100 mL container, tellurium tetrachloride (7.54 g, 28 mmol) was fed, 1.54 g (14 mmol) of resorcinol and 20 ml of carbon tetrachloride were added, and the mixture was reacted at 80° C. for 24 hours under reflux conditions. The obtained reaction solution was washed by the addition of dichloromethane and filtered, and the obtained solid was dried under reduced pressure.

Then, in a 300 ml container, 13.0 g (66 mmol) of sodium ascorbate was dissolved in 25 ml of water, the above solid dissolved in 60 ml of ethyl acetate was dropped to the solution, and the mixture was reacted at 25° C. for 24 hours. The solution after reaction was subjected to extraction with ethyl acetate 15 times, and the organic solvent was then distilled off to obtain a brown solid.

Further, in a container (internal capacity: 100 mL) equipped with a stirrer, a condenser tube, and a burette, the obtained brown solid was placed, 10 ml of ethyl acetate and 13.0 g (60 mmol) of copper powder were added, and the mixture was reacted at 80° C. for 24 hours under reflux conditions. The obtained reaction solution was concentrated 2-fold, the residue was dropped to chloroform, and the obtained precipitates were filtered and dried to obtain 0.2 g of a black-brown resin (Re-co-Te).

As a result of measuring the molecular weight in terms of polystyrene of the obtained resin (Re-co-Te) by the above method, it was Mn: 21500, Mw: 41500, Mw/Mn: 1.93.

The following peaks were found by NMR measurement performed on the obtained resin (Re-co-Te) under the above measurement conditions, and the resin was confirmed to have a chemical structure of the resin (Re-co-Te) shown below.

δ (ppm) 9.1 (2H, —OH), 6.1-7.0 (2H, Ph-H)

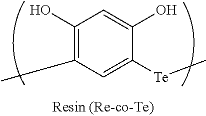

The solubility of the obtained compound in a safe solvent was evaluated by the above method. The results are shown in Table 1.

### (Synthesis Example 37) Synthesis of Resin (DMB-co-TeCl2-ADBAC)

In a container (internal capacity: 200 mL) equipped with a stirrer, a condenser tube, and a burette, 3.7 g of the resin (DMB-co-TeCl2-OH), 0.30 g (22 mmol) of potassium carbonate, and 6.3 g (22 mmol) of bromoacetic acid-2-methyladamantan-2-yl were dissolved in 50 ml of N-methylpyrrolidone, and the solution was stirred for 2 hours. After stirring, 5.7 g (22 mmol) of adamantane bromoacetate was further added thereto, and the mixture was reacted at 100° C. for 24 hours. After the reaction terminated, the reaction mixture was dropped to a 1 N aqueous hydrochloric acid solution, and the resulting black solid was filtered off and dried to obtain 5.3 g of the following resin (DMB-co-TeCl2-ADBAC).

The following peaks were found by NMR measurement performed on the obtained resin (DMB-co-TeCl2-ADBAC) under the above measurement conditions, and the resin was confirmed to have a chemical structure of the resin (DMB-co-TeCl2-ADBAC) shown below.

δ (ppm) 6.5-7.2 (2H, Ph-H), 4.9-5.0 (4H, —CH₂—), 1.0-2.6 (34H, C—H/Adamantane of methylene and methine)

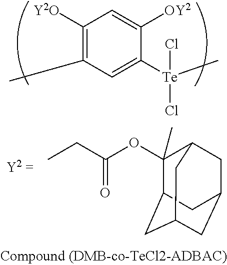

The solubility of the obtained compound in a safe solvent was evaluated by the above method. The results are shown in Table 1.

### (Synthesis Example 38) Synthesis of Resin (Re-co-Te-ADBAC)

In a container (internal capacity: 200 mL) equipped with a stirrer, a condenser tube, and a burette, 2.7 g of the resin (Re-co-Te), 0.30 g (22 mmol) of potassium carbonate, and 0.64 g (2 mmol) of tetrabutyl ammonium bromide were dissolved in 50 ml of N-methylpyrrolidone, and the solution was stirred for 2 hours. After stirring, 6.3 g (22 mmol) of bromoacetic acid-2-methyladamantan-2-yl was further added thereto, and the mixture was reacted at 100° C. for 24 hours. After the reaction terminated, the reaction mixture was dropped to a 1 N aqueous hydrochloric acid solution, and the resulting black solid was filtered off and dried to obtain 4.6 g of the following resin (Re-co-Te-ADBAC).

The following peaks were found by NMR measurement performed on the obtained resin (Re-co-Te-ADBAC) under the above measurement conditions, and the resin was confirmed to have a chemical structure of the resin (Re-co-Te-ADBAC) shown below.

δ (ppm) 6.5-7.2 (2H, Ph-H), 4.9-5.0 (4H, —CH₂—), 1.0-2.6 (34H, C—H/Adamantane of methylene and methine)

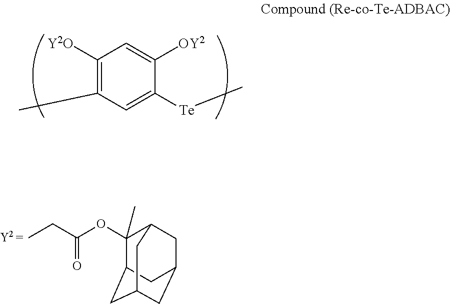

The solubility of the obtained compound in a safe solvent was evaluated by the above method. The results are shown in Table 1.

### (Synthesis Example 39) Synthesis of Resin (DPE-co-Te)

In a glove box, to a 300 ml container, tellurium tetrachloride (75 g, 280 mmol) was fed, 100 ml of carbon tetrachloride and 15 g (140 mmol) of diphenyl ether were added, and the mixture was reacted at 80° C. for 24 hours under reflux conditions. The obtained reaction solution was washed by the addition of dichloromethane and filtered, and the obtained solid was dried under reduced pressure.

Then, in a 1000 ml container, 130 g (66 mmol) of sodium ascorbate was dissolved in 250 ml of water, the above solid dissolved in 120 ml of ethyl acetate was dropped to the solution, and the mixture was reacted at 25° C. for 24 hours. The solution after reaction was subjected to extraction with ethyl acetate 5 times, and the organic solvent was then distilled off to obtain a brown solid.

Further, in a container (internal capacity: 100 mL) equipped with a stirrer, a condenser tube, and a burette, the obtained brown solid was placed, 20 ml of ethyl acetate and 38.0 g (600 mmol) of copper powder were added, and the mixture was reacted at 80° C. for 24 hours under reflux conditions. The obtained reaction solution was concentrated 2-fold, the residue was dropped to hexane, and the obtained precipitates were filtered and dried to obtain 0.11 g of a red resin (DPE-co-Te).

As a result of measuring the molecular weight in terms of polystyrene of the obtained resin (DPE-co-Te) by the above method, it was Mn: 1280, Mw: 2406, Mw/Mn: 1.88.

The following peaks were found by NMR measurement performed on the obtained resin (DPE-co-Te) under the above measurement conditions, and the resin was confirmed to have a chemical structure of the resin (DPE-co-Te) shown below.

δ (ppm) 6.9-8.8 (8H, Ph-H)

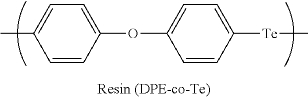

The solubility of the obtained compound in a safe solvent was evaluated by the above method. The results are shown in Table 1.

### (Synthesis Example 40) Synthesis of Tellurium-Containing Core-Shell Type Hyperbranched Polymer

To a 200 mL container, 3.2 g (25 mmol) of tellurium and 25 ml of THF were added and stirred for suspension, 30 ml of a methyllithium solution (1 mol/l, diethyl ether solution) was dropped under ice cooling, and the mixture was stirred at 0° C. for 1 hour. 6.1 g (40 mmol) of chloromethylstyrene was further added thereto, and the mixture was further reacted by being stirred at 25° C. for 2 hours. Next, the solvent in the reaction solution was distilled off, and the residue was dried under reduced pressure to obtain 2.0 g of methyltellanylstyrene.

To a 200 mL container, 3.2 g (25 mmol) of tellurium and 25 ml of THF were added and stirred for suspension, 30 ml of a methyllithium solution (1 mol/l, diethyl ether solution) was dropped under ice cooling, and the mixture was stirred at 0° C. for 1 hour. Next, 20 ml of a 0.5 mol/l aqueous ammonium chloride solution was added thereto, and the mixture was reacted by being stirred at 25° C. for 2 hours. After reaction, the aqueous layer was separated and subjected to extraction with diethyl ether three times. The solvent in the extracted organic layer was distilled off, and the residue was dried under reduced pressure to obtain 2.2 g of dimethyl ditelluride.

Further, to a container (internal capacity: 500 mL) equipped with a stirrer, a condenser tube, and a burette, 80 g of chlorobenzene, 2.6 g (10 mmol) of the above methyltellanylstyrene, 0.7 g (2.5 mmol) of the dimethyl ditelluride, and 0.4 g (2.5 mmol) of azobisisobutyronitrile were added, and the mixture was stirred at 110° C. for 1 hour in the current of nitrogen. After stirring, 90 g of benzene, 0.4 g of acrylic acid, and 4.35 g of t-butyl acrylate were added thereto, and the mixture was further reacted by being stirred at 110° C. for 5 hours. After the reaction terminated, 1500 ml of water was added to the reaction solution, and the mixture was filtered and dried to obtain 2.0 g of a tellurium-containing core-shell type hyperbranched polymer (referred to as “Te-containing hyperbranched polymer” in Table 1).

As a result of measuring the molecular weight in terms of polystyrene of the obtained tellurium-containing core-shell type hyperbranched polymer by the above method, it was Mn: 3260, Mw: 5800, Mw/Mn: 1.78.

The solubility of the obtained compound in a safe solvent was evaluated by the above method. The results are shown in Table 1.

### (Production Example 41) Synthesis of Compound (CCHT)

In a glove box, to a 50 mL container, tellurium tetrachloride (0.27 g, 1.0 mmol) and resorcinol (0.15 g, 1.36 mmol) were fed, 5 mL of carbon tetrachloride was added as a solvent, and the mixture was reacted for 6 hours under reflux conditions. The obtained product was filtered, washed twice with dichloromethane, and dried under reduced pressure to obtain a pale yellow solid. This solid was placed in a 50 mL container, resorcinol (1.10 g, 10 mmol) was added, and then, the mixture was reacted at 170° C. for 24 hours. The obtained reaction solution was dissolved in ethyl acetate, and precipitates were formed again with n-hexane to obtain CCHT ((2,4-dihydroxyphenyl) (4-hydroxyphenyl)tellurium dichloride).

As a result of measuring the molecular weight of the obtained compound (CCHT) by the above measurement method (LC-MS), it was 401.

The following peaks were found by NMR measurement performed on the obtained compound (CCHT) under the above measurement conditions, and the compound was confirmed to have a chemical structure of the compound (CCHT) shown below.

δ (ppm) 9.5-9.9 (3H, —OH), 6.3-7.2 (7H, Ph-H)

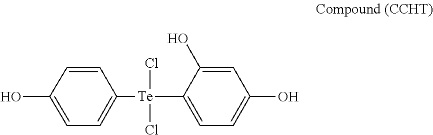

The solubility of the obtained compound in a safe solvent was evaluated by the above method. The results are shown in Table 1.

### (Production Example 42) Synthesis of Compound (CCHT-ADBAC)

The same operations as in Production Example 2 were performed except that 2.7 g (6.7 mmol) of the compound (CCHT) was used in place of 3.9 g (10 mmol) of the compound (BHPT), to obtain 1.09 g of a compound (CCHT-ADBAC) having a structure shown below.

As a result of measuring the molecular weight of the obtained compound (Ph-CCHT-ADBAC) by the above measurement method (LC-MS), it was 537.

The following peaks were found by NMR measurement performed on the obtained compound (CCHT-ADBAC) under the above measurement conditions, and the compound was confirmed to have a chemical structure of the compound (CCHT-ADBAC) shown below.

δ (ppm) 6.5-7.0 (7H, Ph-H), 5.0 (6H, O—CH2-C(═O)—), 1.0-2.6 (51H, C—H/Adamantane of methylene and methine)

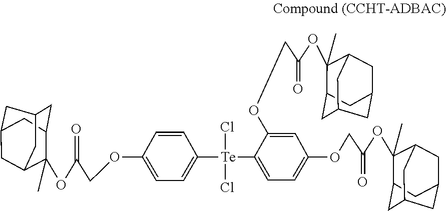

The solubility of the obtained compound in a safe solvent was evaluated by the above method. The results are shown in Table 1.

### Comparative Synthesis Example 1

A 4-neck flask (internal capacity: 10 L) equipped with a Dimroth condenser tube, a thermometer, and a stirring blade and having a detachable bottom was prepared. To this 4-neck flask, 1.09 kg (7 mol) of 1,5-dimethylnaphthalene (manufactured by Mitsubishi Gas Chemical Co., Inc.), 2.1 kg (28 mol as formaldehyde) of 40% by mass of an aqueous formalin solution (manufactured by Mitsubishi Gas Chemical Co., Inc.), and 0.97 mL of 98% by mass of sulfuric acid (manufactured by Kanto Chemical Co., Inc.) were fed in the current of nitrogen, and the mixture was reacted for 7 hours while being refluxed at 100° C. at normal pressure. Subsequently, 1.8 kg of ethylbenzene (a special grade reagent manufactured by Wako Pure Chemical Industries, Ltd.) was added as a diluting solvent to the reaction solution, and the mixture was left to stand still, followed by removal of an aqueous phase as a lower phase. Neutralization and washing with water were further performed, and ethylbenzene and unreacted 1,5-dimethylnaphthalene were distilled off under reduced pressure to obtain 1.25 kg of a dimethylnaphthalene formaldehyde resin as a light brown solid.

The molecular weight of the obtained dimethylnaphthalene formaldehyde was Mn: 562, Mw: 1168, Mw/Mn: 2.08.

Then, a 4-neck flask (internal capacity: 0.5 L) equipped with a Dimroth condenser tube, a thermometer, and a stirring blade was prepared. To this 4-neck flask, 100 g (0.51 mol) of the dimethylnaphthalene formaldehyde resin obtained as described above and 0.05 g of p-toluenesulfonic acid were fed in the current of nitrogen, the temperature was elevated to 190° C., and the mixture was heated for 2 hours and then stirred. Subsequently, 52.0 g (0.36 mol) of 1-naphthol was further added thereto, the temperature was further elevated to 220° C., and the mixture was reacted for 2 hours. After dilution with a solvent, neutralization and washing with water were performed, and the solvent was distilled off under reduced pressure to obtain 126.1 g of a modified resin (CR-1) as a black-brown solid.

The obtained resin (CR-1) had Mn: 885, Mw: 2220, Mw/Mn: 4.17. As a result of evaluating the solubility of the obtained resin (CR-1) in PGMEA by the above measurement method, it was evaluated as 5% by mass or more (evaluation A).

### Examples and Comparative Examples

### (Preparation of Optical Component Forming Composition)

An optical component forming composition was prepared according to the formula shown in Table 1 below using each of the compounds synthesized in Synthesis Examples and Comparative Synthesis Examples. Among the components of the optical component forming composition in Table 1, the following acid generating agent (C), acid crosslinking agent (G), acid diffusion controlling agent (E), and solvent (S-1) were used.

### [Acid Generating Agent (C)]

P-1: triphenylsulfonium trifluoromethanesulfonate

### (Midori Kagaku Co., Ltd.)

### [Acid Crosslinking Agent (G)]

G-I: MX-270 manufactured by Sanwa Chemical

### [Acid Diffusion Controlling Agent (E)]

Q-I: trioctylamine (Tokyo Kasei Kogyo Co., Ltd.)

### [Solvent]

S-1: propylene glycol monomethyl ether acetate (Tokyo Kasei Kogyo Co., Ltd.)

The “storage stabilities” of the obtained optical component forming compositions were evaluated by the above measurement method. Also, the “film formabilities” of the optical component forming compositions in a homogeneous state were evaluated. The obtained results are shown in Table 1.

TABLE 1

| | | | Optical component | | | | | | | | |
|---|---|---|---|---|---|---|---|---|---|---|---|
| | | | forming composition | | | | | | | | |
| | | | | Acid | Acid | | | | | | |
| | | | | gener- | cross- | Acid | | | | | |
| | | | | ating | linking | diffusion | | | | | |
| | | Solubility | | agent | agent | controlling | | | | | |
| | | in | | (C) | (G) | agent | Solvent | Evaluation | | | |
| | | organic | Compound | P-1 | G-1 | (E) | S-1 | Storage | Film | Refractive | Trans- |
| | | solvent | [g] | [g] | [g] | Q-1 [g] | [g] | stability | formability | index | mittance |
| Example 1 | BHPT | A | 0.75 | 0.3 | 0.25 | 0.03 | 25 | A | A | A | A |
| Example 2 | BHPT-ADBAC | A | 0.75 | 0.3 | 0.25 | 0.03 | 25 | A | A | A | A |
| Example 3 | BHPT-BOC | A | 0.75 | 0.3 | 0.25 | 0.03 | 25 | A | A | A | A |
| Example 4 | BHPT-EE | A | 0.75 | 0.3 | 0.25 | 0.03 | 25 | A | A | A | A |
| Example 5 | Ph-BHPT | A | 0.75 | 0.3 | 0.25 | 0.03 | 25 | A | A | A | A |
| Example 6 | TDP | A | 0.75 | 0.3 | 0.25 | 0.03 | 25 | A | A | A | A |
| Example 7 | Ph-TDP | A | 0.75 | 0.3 | 0.25 | 0.03 | 25 | A | A | A | A |
| Example 8 | Ph-BHPT-ADBAC | A | 0.75 | 0.3 | 0.25 | 0.03 | 25 | A | A | A | A |
| Example 9 | TDP-ADBAC | A | 0.75 | 0.3 | 0.25 | 0.03 | 25 | A | A | A | A |
| Example 10 | Ph-TDP-ADBAC | A | 0.75 | 0.3 | 0.25 | 0.03 | 25 | A | A | A | A |
| Example 11 | Ph-TDP-BOC | A | 0.75 | 0.3 | 0.25 | 0.03 | 25 | A | A | A | A |
| Example 12 | Ph-TDP-EE | A | 0.75 | 0.3 | 0.25 | 0.03 | 25 | A | A | A | A |
| Example 13 | R1-BHPT | A | 0.75 | 0.3 | 0.25 | 0.03 | 25 | A | A | A | A |
| Example 14 | R2-BHPT | A | 0.75 | 0.3 | 0.25 | 0.03 | 25 | A | A | A | A |
| Example 15 | R1-BHPT-ADBAC | A | 0.75 | 0.3 | 0.25 | 0.03 | 25 | A | A | A | A |
| Example 16 | R2-BHPT-ADBAC | A | 0.75 | 0.3 | 0.25 | 0.03 | 25 | A | A | A | A |
| Example 17 | R1-BHPT-BOC | A | 0.75 | 0.3 | 0.25 | 0.03 | 25 | A | A | A | A |
| Example 18 | R2-BHPT-BOC | A | 0.75 | 0.3 | 0.25 | 0.03 | 25 | A | A | A | A |
| Example 19 | R1-BHPT-EE | A | 0.75 | 0.3 | 0.25 | 0.03 | 25 | A | A | A | A |
| Example 20 | R2-BHPT-EE | A | 0.75 | 0.3 | 0.25 | 0.03 | 25 | A | A | A | A |
| Example 21 | R1-Ph-BHPT | A | 0.75 | 0.3 | 0.25 | 0.03 | 25 | A | A | A | A |
| Example 22 | R2-Ph-BHPT | A | 0.75 | 0.3 | 0.25 | 0.03 | 25 | A | A | A | A |
| Example 23 | R1-TDP | A | 0.75 | 0.3 | 0.25 | 0.03 | 25 | A | A | A | A |
| Example 24 | R2-TDP | A | 0.75 | 0.3 | 0.25 | 0.03 | 25 | A | A | A | A |
| Example 25 | R1-Ph-TDP | A | 0.75 | 0.3 | 0.25 | 0.03 | 25 | A | A | A | A |
| Example 26 | R2-Ph-TDP | A | 0.75 | 0.3 | 0.25 | 0.03 | 25 | A | A | A | A |
| Example 27 | R1-Ph-BHPT-ADBAC | A | 0.75 | 0.3 | 0.25 | 0.03 | 25 | A | A | A | A |
| Example 28 | R2-Ph-BHPT-ADBAC | A | 0.75 | 0.3 | 0.25 | 0.03 | 25 | A | A | A | A |
| Example 29 | R1-TDP-ADBAC | A | 0.75 | 0.3 | 0.25 | 0.03 | 25 | A | A | A | A |
| Example 30 | R2-TDP-ADBAC | A | 0.75 | 0.3 | 0.25 | 0.03 | 25 | A | A | A | A |
| Example 31 | R1-Ph-TDP-ADBAC | A | 0.75 | 0.3 | 0.25 | 0.03 | 25 | A | A | A | A |
| Example 32 | R2-Ph-TDP-ADBAC | A | 0.75 | 0.3 | 0.25 | 0.03 | 25 | A | A | A | A |
| Example 33 | BHPT-co-ADTBA | A | 0.75 | 0.3 | 0.25 | 0.03 | 25 | A | A | A | A |
| Example 34 | TDP-co-ADTBA | A | 0.75 | 0.3 | 0.25 | 0.03 | 25 | A | A | A | A |
| Example 35 | DMB-co-TeCl2-OH | A | 0.75 | 0.3 | 0.25 | 0.03 | 25 | A | A | A | A |
| Example 36 | Re-co-Te | A | 0.75 | 0.3 | 0.25 | 0.03 | 25 | A | A | A | A |
| Example 37 | DMB-co-TeCl2-ADBAC | A | 0.75 | 0.3 | 0.25 | 0.03 | 25 | A | A | A | A |
| Example 38 | Re-co-Te-ADBAC | A | 0.75 | 0.3 | 0.25 | 0.03 | 25 | A | A | A | A |
| Example 39 | DPE-co-Te | A | 0.75 | 0.3 | 0.25 | 0.03 | 25 | A | A | A | A |
| Example 40 | Te-containing | A | 0.75 | 0.3 | 0.25 | 0.03 | 25 | A | A | A | A |
| | hyperbranched | | | | | | | | | | |
| | polymer | | | | | | | | | | |
| Example 41 | CCHT | A | 0.75 | 0.3 | 0.25 | 0.03 | 25 | A | A | A | A |
| Example 42 | CCHT-ADBAC | A | 0.75 | 0.3 | 0.25 | 0.03 | 25 | A | A | A | A |
| Example 43 | BHPT | A | 1.2 | 0 | 0 | 0 | 25 | A | A | A | A |
| Example 44 | BHPT-ADBAC | A | 1.2 | 0 | 0 | 0 | 25 | A | A | A | A |
| Example 45 | TDP | A | 1.2 | 0 | 0 | 0 | 25 | A | A | A | A |
| Example 46 | R1-BHPT | A | 1.2 | 0 | 0 | 0 | 25 | A | A | A | A |
| Example 47 | R1-BHPT-ADBAC | A | 1.2 | 0 | 0 | 0 | 25 | A | A | A | A |
| Example 48 | Re-co-Te | A | 1.2 | 0 | 0 | 0 | 25 | A | A | A | A |
| Comparative | CR-1 | A | 0.75 | 0.3 | 0.25 | 0.03 | 25 | A | A | C | C |
| Example 1 | | | | | | | | | | | |

As can be understood from Table 1, the compounds used in Examples 1 to 48 were able to be confirmed to have good solubility.

In the stability evaluation, the optical component forming compositions obtained in Examples 1 to 48 were confirmed to have good storage stability without precipitates (evaluation: A).

As a result of evaluating film formability according to the above measurement method, the optical component forming compositions obtained in Examples 1 to 48 were able to form an excellent film.

From the above results, it was found that the compounds meeting the requirements of the present invention have high solubility in organic solvents, and, also, optical component forming compositions containing the compounds have good storage stability and film formability and can confer high refractive index and high transmittance. As long as the above requirements of the present invention are met, compounds other than the compounds described in Examples also exhibit the same effects.

### INDUSTRIAL APPLICABILITY

The optical component forming composition of the present invention contains a compound having a specific structure and having high solubility in organic solvents, has good storage stability and film formability, and can impart high refractive index to a structure. Accordingly, the present invention is useful in the optical component field and the like where optical component forming compositions having high refractive index are used.

The disclosure of Japanese Patent Application No. 2016-091792 filed on Apr. 28, 2016 is incorporated herein by reference in its entirety.

All literatures, patent applications, and technical standards described herein are incorporated herein by referent to the same extent as if each individual literature, patent application, or technical standard is specifically and individually indicated to be incorporated by reference.

1. An optical component forming composition comprising a tellurium-containing compound or a tellurium-containing resin.

2. The optical component forming composition according to claim 1, wherein the tellurium-containing compound is represented by the following formula (A-1): 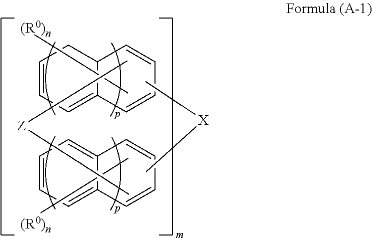
wherein X is a 2m-valent group of 0 to 60 carbon atoms containing tellurium; Z is an oxygen atom, a sulfur atom, or non-crosslinked state; each R⁰ is independently selected from the group consisting of a monovalent group containing an oxygen atom, a monovalent group containing a sulfur atom, a monovalent group containing a nitrogen atom, a hydrocarbon group, a halogen atom, and a combination thereof; m is an integer of 1 to 4; each p is independently an integer of 0 to 2; and each n is independently an integer of 0 to (5+2×p).

3. The optical component forming composition according to claim 2, wherein the tellurium-containing compound is represented by the following formula (A-2): 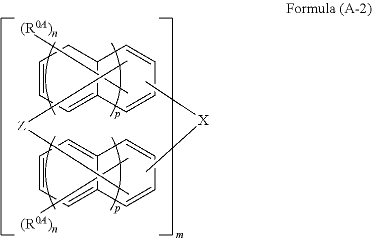
wherein X is a 2m-valent group of 0 to 60 carbon atoms containing tellurium; Z is an oxygen atom, a sulfur atom, a single bond, or non-crosslinked state; each R^{0A} is independently selected from the group consisting of a hydrocarbon group, a halogen atom, a cyano group, a nitro group, an amino group, an alkyl group of 1 to 30 carbon atoms, an alkenyl group of 2 to 30 carbon atoms, an aryl group of 6 to 40 carbon atoms, a hydroxy group or a group in which a hydrogen atom of a hydroxy group is substituted with an acid crosslinking reactive group or an acid dissociation reactive group, and a combination thereof, wherein the alkyl group, the alkenyl group, and the aryl group each optionally have an ether bond, a ketone bond, or an ester bond; m is an integer of 1 to 4; each p is independently an integer of 0 to 2; and each n is independently an integer of 0 to (5+2×p).

4. The optical component forming composition according to claim 2, wherein the tellurium-containing compound is represented by the following formula (A-3): 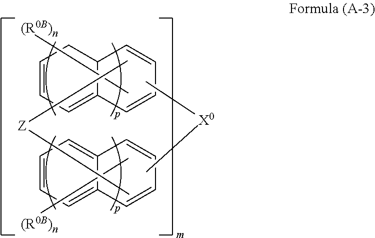
wherein X⁰ is a 2m-valent group of 0 to 30 carbon atoms containing tellurium; Z is an oxygen atom, a sulfur atom, or non-crosslinked state; each R^{0B} is independently a monovalent group containing an oxygen atom, a monovalent group containing a sulfur atom, a monovalent group containing a nitrogen atom, a hydrocarbon group, or a halogen atom; m is an integer of 1 to 4; each p is independently an integer of 0 to 2; and each n is independently an integer of 0 to (5+2×p).

5. The optical component forming composition according to claim 2, wherein the tellurium-containing compound is represented by the following formula (1A): 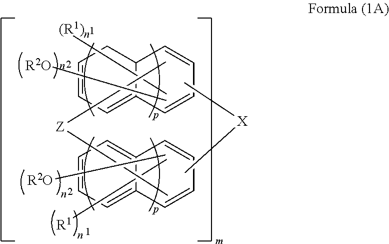
wherein X, Z, m, and p are as defined in the above formula (A-1); each R¹ is independently selected from the group consisting of a hydrocarbon group, a halogen atom, a cyano group, a nitro group, an amino group, an alkyl group of 1 to 30 carbon atoms, an alkenyl group of 2 to 30 carbon atoms, an aryl group of 6 to 40 carbon atoms, and a combination thereof, wherein the alkyl group, the alkenyl group, and the aryl group each optionally have an ether bond, a ketone bond, or an ester bond; each R² is independently a hydrogen atom, an acid crosslinking reactive group, or an acid dissociation reactive group; each n¹ is independently an integer of 0 to (5+2×p); and each n² is independently an integer of 0 to (5+2×p), provided that at least one n² is an integer of 1 to (5+2×p).

6. The optical component forming composition according to claim 4, wherein the tellurium-containing compound is represented by the following formula (1B): 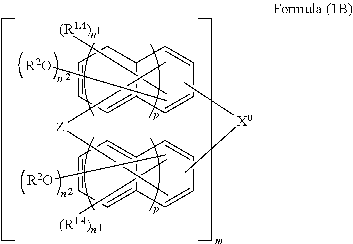
wherein X⁰, Z, m, and p are as defined in the above formula (A-3); each R^{1A} is independently an alkyl group, an aryl group, an alkenyl group, or a halogen atom; each R² is independently a hydrogen atom, an acid crosslinking reactive group, or an acid dissociation reactive group; each n¹ is independently an integer of 0 to (5+2×p); and each n² is independently an integer of 0 to (5+2×p), provided that at least one n² is an integer of 1 to (5+2×p).

7. The optical component forming composition according to claim 6, wherein the tellurium-containing compound is represented by the following formula (2A): 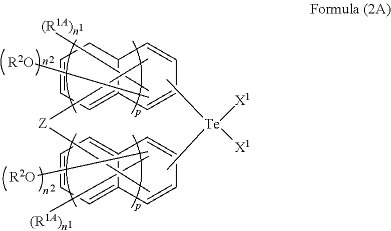
wherein Z, R^{1A}, R², p, n¹, and n² are as defined in the above formula (1B); and each X¹ is independently a monovalent group containing an oxygen atom, a monovalent group containing a sulfur atom, a monovalent group containing a nitrogen atom, a hydrocarbon group, a hydrogen atom, or a halogen atom.

8. The optical component forming composition according to claim 7, wherein the tellurium-containing compound is represented by the following formula (2A′): 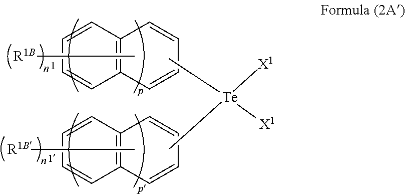
wherein R^{1B} and R^{1B′} are each independently an alkyl group, an aryl group, an alkenyl group, a halogen atom, a hydroxy group or a group in which a hydrogen atom of a hydroxy group is substituted with an acid crosslinking reactive group or an acid dissociation reactive group; X¹ is as defined as X¹ in the above formula (2A); n¹ and n^{1′} are as defined as n¹ in the above formula (2A); p and p′ are as defined as p in the above formula (2A); and members in at least one combination selected from R^{1B} and R^{1B′}, n¹ and n^{1′}, p and p′, and the substitution position of R^{1B} and the substitution position of R^{1B′} differ from each other.

9. The optical component forming composition according to claim 7, wherein the tellurium-containing compound is represented by the following formula (3A): 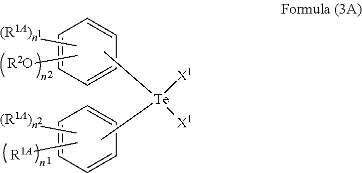
wherein R^{1A}, R², X¹, n¹, and n² are as defined in the above formula (2A).

10. The optical component forming composition according to claim 9, wherein the tellurium-containing compound is represented by the following formula (4A): 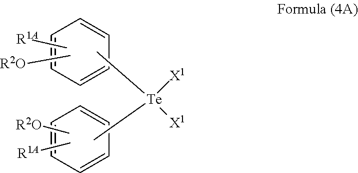
wherein R^{1A}, R², and X¹ are as defined in the above formula (3A).

11. The optical component forming composition according to claim 6, wherein the tellurium-containing compound is represented by the following formula (2B): 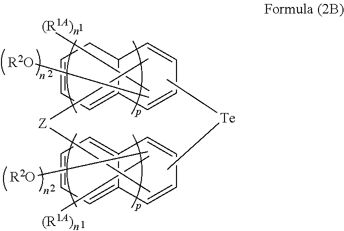
wherein Z, R^{1A}, R², p, n¹, and n² are as defined in the above formula (1B).

12. The optical component forming composition according to claim 11, wherein the tellurium-containing compound is represented by the following formula (2B′): 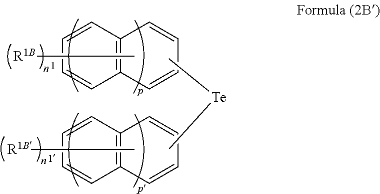
wherein R^{1B} and R^{1B′} are each independently an alkyl group, an aryl group, an alkenyl group, a halogen atom, a hydroxy group or a group in which a hydrogen atom of a hydroxy group is substituted with an acid crosslinking reactive group or an acid dissociation reactive group; n¹ and n^{1′} are as defined as n¹ in the above formula (2B); p and p′ are as defined as p in the above formula (2B); and members in at least one combination selected from R^{1B} and R^{1B′}, n¹ and n^{1′}, p and p′, and the substitution position of R^{1B} and the substitution position of R^{1B′} differ from each other.

13. The optical component forming composition according to claim 11, wherein the tellurium-containing compound is represented by the following formula (3B): 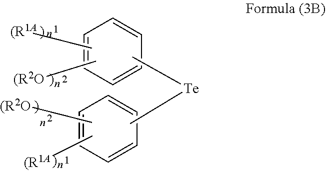
wherein R^{1A}, R², n¹, and n² are as defined in the above formula (2B).

14. The optical component forming composition according to claim 13, wherein the tellurium-containing compound is represented by the following formula (4B): 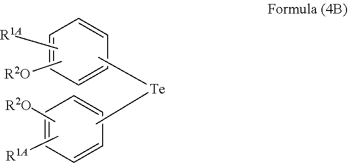
wherein R¹, R², and X¹ are as defined in the above formula (3B).

15. The optical component forming composition according to claim 5, wherein the tellurium-containing compound has at least one acid dissociation reactive group as the R².

16. The optical component forming composition according to claim 5, wherein all of the R² in the tellurium-containing compound are hydrogen atoms.

17. The optical component forming composition according to claim 1, wherein the tellurium-containing resin is a resin comprising a constitutional unit derived from a compound represented by the following formula (A-1): 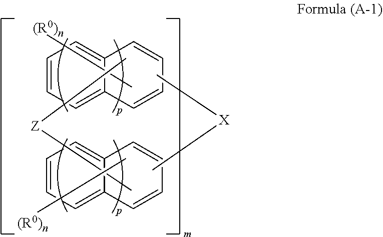
wherein X is a 2m-valent group of 0 to 60 carbon atoms containing tellurium; Z is an oxygen atom, a sulfur atom, or non-crosslinked state; each R⁰ is independently selected from the group consisting of a monovalent group containing an oxygen atom, a monovalent group containing a sulfur atom, a monovalent group containing a nitrogen atom, a hydrocarbon group, a halogen atom, and a combination thereof; m is an integer of 1 to 4; each p is independently an integer of 0 to 2; and each n is independently an integer of 0 to (5+2×p).

18. The optical component forming composition according to claim 1, wherein the tellurium-containing resin is a resin comprising a constitutional unit derived from a compound represented by the following formula (A-2): 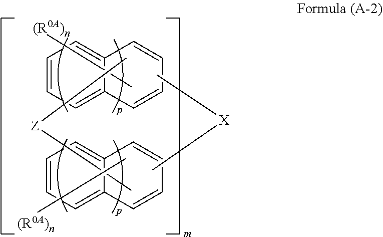
wherein X is a 2m-valent group of 0 to 60 carbon atoms containing tellurium; Z is an oxygen atom, a sulfur atom, a single bond, or non-crosslinked state; each R^{0A} is independently selected from the group consisting of a hydrocarbon group, a halogen atom, a cyano group, a nitro group, an amino group, an alkyl group of 1 to 30 carbon atoms, an alkenyl group of 2 to 30 carbon atoms, an aryl group of 6 to 40 carbon atoms, a hydroxy group or a group in which a hydrogen atom of a hydroxy group is substituted with an acid crosslinking reactive group or an acid dissociation reactive group, and a combination thereof, wherein the alkyl group, the alkenyl group, and the aryl group each optionally have an ether bond, a ketone bond, or an ester bond; m is an integer of 1 to 4; each p is independently an integer of 0 to 2; and each n is independently an integer of 0 to (5+2×p).

19. The optical component forming composition according to claim 1, wherein the tellurium-containing resin is a resin comprising a constitutional unit derived from a compound represented by the following formula (A-3): 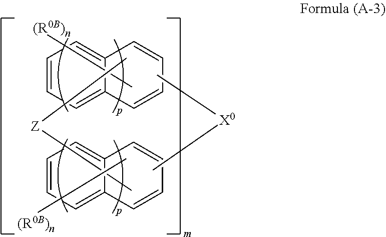
wherein X⁰ is a 2m-valent group of 0 to 30 carbon atoms containing tellurium; Z is an oxygen atom, a sulfur atom, or non-crosslinked state; each R^{0B} is independently a monovalent group containing an oxygen atom, a monovalent group containing a sulfur atom, a monovalent group containing a nitrogen atom, a hydrocarbon group, or a halogen atom; m is an integer of 1 to 4; each p is independently an integer of 0 to 2; and each n is independently an integer of 0 to (5+2×p).

20. The optical component forming composition according to claim 1, wherein the tellurium-containing resin is a resin comprising a constitutional unit represented by the following formula (B1-M): 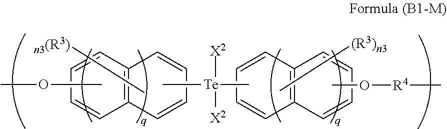
wherein each X² is independently a monovalent group containing an oxygen atom, a monovalent group containing a sulfur atom, a monovalent group containing a nitrogen atom, a hydrocarbon group, a hydrogen atom, or a halogen atom; each R³ is independently a monovalent group containing an oxygen atom, a monovalent group containing a sulfur atom, a monovalent group containing a nitrogen atom, a hydrocarbon group, or a halogen atom; q is an integer of 0 to 2; n³ is an integer of 0 to (4+2×q); and R⁴ is a single bond or any structure represented by the following general formula (5): 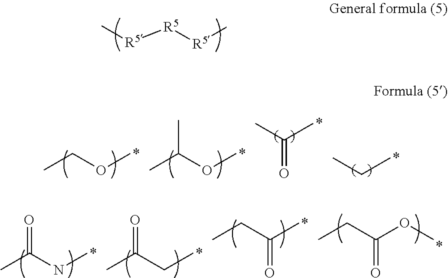
wherein R⁵ is a substituted or unsubstituted linear alkylene group of 1 to 20 carbon atoms, branched alkylene group of 3 to 20 carbon atoms, or cyclic alkylene group of 3 to 20 carbon atoms, or a substituted or unsubstituted arylene group of 6 to 20 carbon atoms; and each R^{5′} is independently any structure of the above formula (5′) wherein * indicates that this portion is connected to R⁵.

21. The optical component forming composition according to claim 20, wherein the R⁴ in the tellurium-containing resin is any structure represented by the above general formula (5).

22. The optical component forming composition according to claim 20, wherein the tellurium-containing resin is a resin comprising a constitutional unit represented by the following formula (B2-M′): 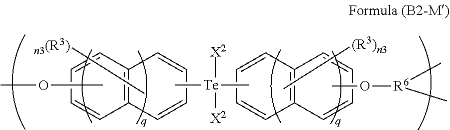
wherein X², R³, q, and n³ are as defined in the formula (B1-M); and R⁶ is any structure represented by the following general formula (6): 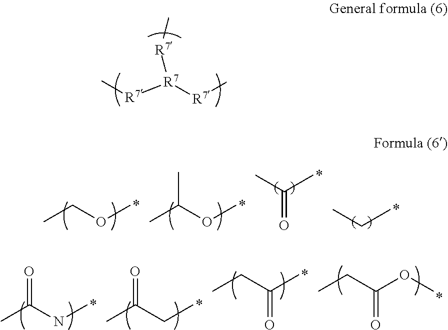
wherein R⁷ is a substituted or unsubstituted linear alkylene group of 1 to 20 carbon atoms, branched alkylene group of 3 to 20 carbon atoms, or cyclic alkylene group of 3 to 20 carbon atoms, or a substituted or unsubstituted arylene group of 6 to 20 carbon atoms; each R^{7′} is independently any structure of the above formula (6′) wherein * indicates that this portion is connected to R⁷.

23. The optical component forming composition according to claim 1, wherein the tellurium-containing resin is a resin comprising a constitutional unit represented by the following formula (C1): 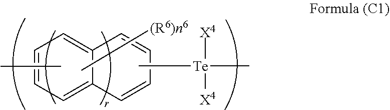
wherein each X⁴ is independently a monovalent group containing an oxygen atom, a monovalent group containing a sulfur atom, a monovalent group containing a nitrogen atom, a hydrocarbon group, a hydrogen atom, or a halogen atom; each R⁶ is independently a monovalent group containing an oxygen atom, a monovalent group containing a sulfur atom, a monovalent group containing a nitrogen atom, a hydrocarbon group, or a halogen atom; r is an integer of 0 to 2; and n⁶ is an integer of 2 to (4+2×r).

24. The optical component forming composition according to claim 1, wherein the tellurium-containing resin is a resin comprising a constitutional unit represented by the following formula (B3-M): 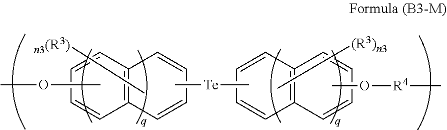
wherein each R³ is independently a monovalent group containing an oxygen atom, a monovalent group containing a sulfur atom, a monovalent group containing a nitrogen atom, a hydrocarbon group, or a halogen atom; q is an integer of 0 to 2; n³ is an integer of 0 to (4+2×q); and R⁴ is a single bond or any structure represented by the following general formula (5): 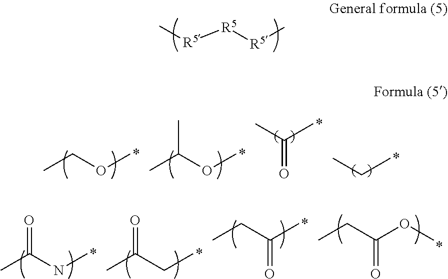
wherein R⁵ is a substituted or unsubstituted linear alkylene group of 1 to 20 carbon atoms, branched alkylene group of 3 to 20 carbon atoms, or cyclic alkylene group of 3 to 20 carbon atoms, or a substituted or unsubstituted arylene group of 6 to 20 carbon atoms; each R5′ is independently any structure of the above formula (5′) wherein * indicates that this portion is connected to R⁵.

25. The optical component forming composition according to claim 24, wherein the R⁴ in the tellurium-containing resin is any structure represented by the above general formula (5).

26. The optical component forming composition according to claim 24, wherein the tellurium-containing resin is a resin comprising a constitutional unit represented by the following formula (B4-M′): 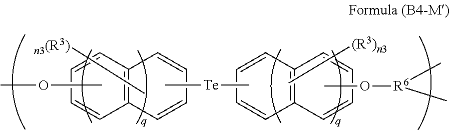
wherein R³, q, and n³ are as defined in the formula (B3-M); and R⁶ is any structure represented by the following general formula (6): 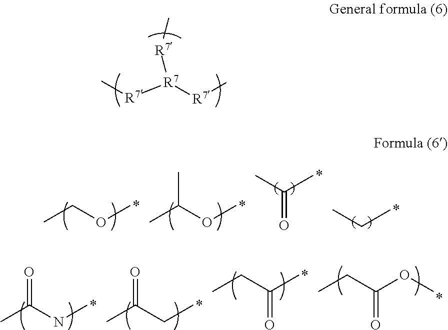
wherein R⁷ is a substituted or unsubstituted linear alkylene group of 1 to 20 carbon atoms, branched alkylene group of 3 to 20 carbon atoms, or cyclic alkylene group of 3 to 20 carbon atoms, or a substituted or unsubstituted arylene group of 6 to 20 carbon atoms; and each R^{7′} is independently any structure of the above formula (6′) wherein * indicates that this portion is connected to R⁷.

27. The optical component forming composition according to claim 1, wherein the tellurium-containing resin is a resin comprising a constitutional unit represented by the following formula (C2): 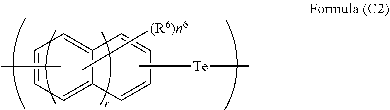
wherein each R⁶ is independently a monovalent group containing an oxygen atom, a monovalent group containing a sulfur atom, a monovalent group containing a nitrogen atom, a hydrocarbon group, or a halogen atom; r is an integer of 0 to 2; and n⁶ is an integer of 2 to (4+2×r).

28. A method for producing the optical component forming composition according to claim 1, comprising the step of reacting a tellurium halide with a substituted or unsubstituted phenol derivative in the presence of a basic catalyst to synthesize the tellurium-containing compound.

29. The optical component forming composition according to claim 1, further comprising a solvent.

30. The optical component forming composition according to claim 29, further comprising an acid generating agent.

31. The optical component forming composition according to claim 29, further comprising an acid crosslinking agent.

32. A cured product obtained using the optical component forming composition according to claim 1.
